(19) United States
(12) Patent Application Publication
(10) Pub. No.: US 2024/0115675 A1
(43) Pub. Date: Apr. 11, 2024

(54) METHOD OF TREATING A TUMOR WITH A COMBINATION OF AN IL-7 PROTEIN AND A NUCLEOTIDE VACCINE

(71) Applicants: NeoImmuneTech, Inc., Rockville, MD (US); Washington University, St. Louis, MO (US)

(72) Inventors: Byung Ha LEE, Rockville, MD (US); Donghoon CHOI, Seongnam-si (KR); William GILLANDERS, St. Louis, MO (US); Ina CHEN, St. Louis, MO (US); Simon Peter GOEDEGEBUURE, St. Louis, MO (US); Lijin LI, St. Louis, MO (US)

(73) Assignees: NeoImmuneTech, Inc., Rockville, MD (US); Washington University, St. Louis, MO (US)

(21) Appl. No.: 18/251,823

(22) PCT Filed: Nov. 5, 2021
(86) PCT No.: PCT/US2021/058273

§ 371 Date: May 4, 2023

Related U.S. Application Data
(60) Provisional application No. 63/110,142, filed on Nov. 5, 2020.

(51) Int. Cl. A61K 39/00

(52) U.S. Cl. CPC A61K 39/0011; A61K 39/00114; A61K 2039/53; A61K 2039/545; A61K 2039/55527

(57) ABSTRACT

The present disclosure relates to methods of treating a tumor with a nucleotide vaccine (e.g., DNA vaccine encoding a tumor antigen) in combination with an IL-7. In some aspects, the IL-7 is administered after the administration of the nucleotide vaccine (e.g., after the peak expansion phase of the tumor-specific T cell immune response) or concurrently with the nucleotide vaccine.

### CROSS-REFERENCE TO RELATED APPLICATIONS

This PCT application claims the priority benefit of U.S. Provisional Application No. 63/110,142, filed Nov. 5, 2020, which is incorporated herein by reference in its entirety.

### REFERENCE TO SEQUENCE LISTING SUBMITTED ELECTRONICALLY VIA EFS-WEB

The content of the electronically submitted sequence listing in ASCII text file (Name: 4241_017PC01_SeqListing_ST25.txt; Size: 83,472 bytes; and Date of Creation: Nov. 5, 2021) filed with the application is herein incorporated by reference in its entirety.

### BACKGROUND OF THE DISCLOSURE

Cancer remains one of the leading causes of death in the modern world. The standard treatments currently practiced in the clinic, including surgery, radiation, chemotherapy, and immunotherapy, have shown limited success. These therapies are usually only effective against early stage localized tumors and rarely against later staged, metastatic malignancies, leading to frequent relapse or eventual resistance to the therapy. Sharma, P., et al., Cell 168(4): 707-723 (2017). Furthermore, various agents used in radiation and chemotherapy are damaging to normal tissues, which can lead to undesirable side effects. Accordingly, there remains a need for new treatment options with acceptable safety profile and high efficacy in cancer patients.

### BRIEF SUMMARY OF THE DISCLOSURE

Disclosed herein is a method of treating a tumor in a subject in need thereof, comprising administering to the subject a nucleotide vaccine encoding a tumor antigen in combination with an interleukin-7 (IL-7), wherein the administration of the nucleotide vaccine induces a tumor-specific T cell immune response, and wherein the IL-7 is administered to the subject within about 14 days, within about 13 days, within about 12 days, within about 11 days, within about 10 days, within about nine days, within about eight days, within about seven days, within about six days, within about five days, within about four days, within about three days, within about two days, or within about one day of the nucleotide vaccine administration. In some aspects, the IL-7 is administered within about seven days of the nucleotide vaccine administration. In some aspects, the IL-7 and the nucleotide vaccine are administered to the subject concurrently.

Disclosed herein is a method of treating a tumor in a subject in need thereof, comprising administering to the subject a nucleotide vaccine encoding a tumor antigen in combination with an interleukin-7 (IL-7), wherein the administration of the nucleotide vaccine induces a tumor-specific T cell immune response, and wherein the IL-7 is administered to the subject after a peak expansion phase of the tumor-specific T cell immune response.

In some aspects, a tumor volume is reduced in the subject after the administration compared to a reference (e.g., corresponding value in a subject that received either IL-7 alone or nucleotide vaccine alone). In certain aspects, the tumor volume is reduced by at least about 5%, at least about 10%, at least about 20%, at least about 30%, at least about 40%, at least about 50%, at least about 60%, at least about 70%, at least about 80%, at least about 90%, or about 100% after the administration compared to the reference.

Provided herein is also a method of preventing or reducing the occurrence of a tumor in a subject in need thereof, comprising administering to the subject a nucleotide vaccine encoding a tumor antigen in combination with an interleukin-7 (IL-7), wherein the administration of the nucleotide vaccine induces a tumor-specific T cell immune response, and wherein the nucleotide vaccine, the IL-7, or both the nucleotide vaccine and the IL-7 are administered to the subject prior to the occurrence of the tumor.

In some aspects, the IL-7 is administered to the subject within about 14 days, within about 13 days, within about 12 days, within about 11 days, within about 10 days, within about nine days, within about eight days, within about seven days, within about six days, within about five days, within about four days, within about three days, within about two days, or within about one day of the nucleotide vaccine administration. In some aspects, the IL-7 is administered to the subject within about seven days of the nucleotide vaccine administration. In some aspects, the IL-7 and the nucleotide vaccine are administered to the subject concurrently.

Also provided herein is a method of prolonging a tumor-specific T cell immune response in a subject in need thereof, comprising administering to the subject a nucleotide vaccine encoding a tumor antigen in combination with an interleukin-7 (IL-7), wherein the administration of the nucleotide vaccine induces a tumor-specific T cell immune response, and wherein the IL-7 is administered to the subject after a peak expansion phase of the tumor-specific T cell immune response.

Present disclosure further provides a method of prolonging a tumor-specific T cell immune response in a subject in need thereof, comprising administering to the subject a nucleotide vaccine encoding a tumor antigen in combination with an interleukin-7 (IL-7), wherein the administration of the nucleotide vaccine induces a tumor-specific T cell immune response, and wherein the IL-7 is administered to the subject within about 14 days, within about 13 days, within about 12 days, within about 11 days, within about 10 days, within about nine days, within about eight days, within about seven days, within about six days, within about five days, within about four days, within about three days, within about two days, or within about one day of the nucleotide vaccine administration. In some aspects, the IL-7 is administered within about seven days of the nucleotide vaccine administration. In some aspects, the IL-7 and the nucleotide vaccine are administered to the subject concurrently.

In some aspects, the administration of IL-7 increases a survival of tumor-specific T cells during a contraction phase of the tumor-specific T cell immune response, compared to a reference (e.g., corresponding value in a subject that received either IL-7 alone or nucleotide vaccine alone). In certain aspects, the survival of tumor-specific T cells during the contraction phase is increased by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 35-fold, at least about 40-fold, at least about 45-fold, or at least about 50-fold or more, compared to the reference.

In some aspects, the administration of IL-7 increases a number of tumor-specific T cells during a contraction phase of the tumor-specific T cell immune response, compared to a reference (e.g., corresponding value in a subject that received either IL-7 alone or nucleotide vaccine alone). In certain aspects, the number of tumor-specific T cells during the contraction phase of the tumor-specific T cell immune response is increased by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 35-fold, at least about 40-fold, at least about 45-fold, or at least about 50-fold or more, compared to the reference.

Provided herein is a method of expanding a T-cell receptor (TCR) repertoire of a tumor-specific T cell immune response in a subject in need thereof, comprising administering to the subject a nucleotide vaccine encoding a tumor antigen in combination with an interleukin-7 (IL-7), wherein the administration of the nucleotide vaccine induces a tumor-specific T cell immune response against one or more epitopes of the tumor antigen, and wherein the IL-7 is administered to the subject after a peak expansion phase of the tumor-specific T cell immune response.

Also provided herein is a method of expanding a T-cell receptor (TCR) repertoire of a tumor-specific T cell immune response in a subject in need thereof, comprising administering to the subject a nucleotide vaccine encoding a tumor antigen in combination with an interleukin-7 (IL-7), wherein the administration of the nucleotide vaccine induces a tumor-specific T cell immune response against one or more epitopes of the tumor antigen, and wherein the IL-7 is administered to the subject within about 14 days, within about 13 days, within about 12 days, within about 11 days, within about 10 days, within about nine days, within about eight days, within about seven days, within about six days, within about five days, within about four days, within about three days, within about two days, or within about one day of the nucleotide vaccine administration. In some aspects, the IL-7 is administered within about seven days of the nucleotide vaccine administration. In some aspects, the IL-7 and the nucleotide vaccine are administered to the subject concurrently.

In some aspects, the administration increases the number of epitopes against which the tumor-specific T cell immune response is induced, compared to a reference (e.g., corresponding value in a subject that received either IL-7 alone or nucleotide vaccine alone). In certain aspects, the number of epitopes against which the tumor-specific T cell immune response is induced is increased by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 35-fold, at least about 40-fold, at least about 45-fold, or at least about 50-fold or more, compared to the reference.

In some aspects, the tumor antigen is derived from a breast cancer and the epitopes are selected from Lrrc27, Plekho1, Pttg1, Xpo4, Exoc4, Pank3, Tmem101, Map3k6, Met, BC057079, Hist1h3e, Prkag1, Neil3, or combinations thereof.

In some aspects, the administration induces a tumor-specific T cell immune response to at least about three, at least about four, at least about five, at least about six, at least about seven, at least about eight, at least about nine, at least about 10, at least about 11, at least about 12, or at least about 13 or more epitopes on the tumor antigen.

Present disclosure further provides a method of increasing a T cell immune response against a subdominant epitope of a tumor antigen in a subject in need thereof, comprising administering to the subject a nucleotide vaccine encoding a tumor antigen, which comprises the subdominant epitope, in combination with an interleukin-7 (IL-7), wherein the IL-7 is administered to the subject after a peak expansion phase of the tumor-specific T cell immune response.

Provided herein is also a method of increasing a T cell immune response against a subdominant epitope of a tumor antigen in a subject in need thereof, comprising administering to the subject a nucleotide vaccine encoding a tumor antigen, which comprises the subdominant epitope, in combination with an interleukin-7 (IL-7), wherein the IL-7 is administered to the subject within about 14 days, within about 13 days, within about 12 days, within about 11 days, within about 10 days, within about nine days, within about eight days, within about seven days, within about six days, within about five days, within about four days, within about three days, within about two days, or within about one day of the nucleotide vaccine administration. In some aspects, the IL-7 is administered within about seven days of the nucleotide vaccine administration. In some aspects, the IL-7 and the nucleotide vaccine are administered to the subject concurrently.

In some aspects, a T cell immune response against a subdominant epitope of a tumor antigen is increased by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 35-fold, at least about 40-fold, at least about 45-fold, or at least about 50-fold or more, compared to a reference (e.g., corresponding value in a subject that received an IL-7 alone or nucleotide vaccine alone).

In any of the above methods, in some aspects, the peak expansion phase of the tumor-specific T cell immune response occurs at about seven days, about eight days, about nine days, about 10 days, about 11 days, about 12 days, about 13 days, about 14 days, or about 15 days after an initial administration of the nucleotide vaccine. In some aspects, the peak expansion phase of the tumor-specific T cell immune response occurs at about 11 days after an initial administration of the nucleotide vaccine.

In the methods disclosed herein, in some aspects, the IL-7 is administered at least about one day, about two days, about three days, about four days, about five days, about six days, about seven days, about eight days, about nine days, about 10 days, about 11 days, about 12 days, about 13 days, about 14 days, about 15 days, about 16 days, about 17 days, about 18 days, about 19 days, about 20 days, about 21 days, about 22 days, about 23 days, about 24 days, about 25 days, about 26 days, about 27 days, about 28 days, about 29 days, or about 30 days or more after the peak expansion phase of the tumor-specific T cell immune response. In certain aspects, the IL-7 is administered at about two days after the peak expansion phase of the tumor-specific T cell immune response.

In some aspects, the IL-7 is administered at a dose between about 5 mg/kg and about 15 mg/kg. In some aspects, the IL-7 is administered at a dose of about 5 mg/kg. In some aspects, the IL-7 is administered at a dose of between about 20 μg/kg and about 600 μg/kg. In certain aspects, the IL-7 protein is administered at a dose of about 20 μg/kg, about 60 μg/kg, about 120 μg/kg, about 240 μg/kg, about 480 μg/kg, or about 600 μg/kg.

In some aspects, the IL-7 protein is administered at a dose greater than about 600 μg/kg, greater than about 700 μg/kg, greater than about 800 μg/kg, greater than about 900 μg/kg, greater than about 1,000 μg/kg, greater than about 1,100 μg/kg, greater than about 1,200 μg/kg, greater than about 1,300 μg/kg, greater than about 1,400 μg/kg, greater than about 1,500 μg/kg, greater than about 1,600 μg/kg, greater than about 1,700 μg/kg, greater than about 1,800 μg/kg, greater than about 1,900 μg/kg, or greater than about 2,000 μg/kg.

In some aspects, the IL-7 protein is administered at a dose of between about 610 μg/kg and about 1,200 μg/kg, between about 650 μg/kg and about 1,200 μg/kg, between about 700 μg/kg and about 1,200 μg/kg, between about 750 μg/kg and about 1,200 μg/kg, between about 800 μg/kg and about 1,200 μg/kg, between about 850 μg/kg and about 1,200 μg/kg, between about 900 μg/kg and about 1,200 μg/kg, between about 950 μg/kg and about 1,200 μg/kg, between about 1,000 μg/kg and about 1,200 μg/kg, between about 1,050 μg/kg and about 1,200 μg/kg, between about 1,100 μg/kg and about 1,200 μg/kg, between about 1,200 μg/kg and about 2,000 μg/kg, between about 1,300 μg/kg and about 2,000 μg/kg, between about 1,500 g/kg and about 2,000 μg/kg, between about 1,700 μg/kg and about 2,000 μg/kg, between about 610 μg/kg and about 1,000 μg/kg, between about 650 μg/kg and about 1,000 μg/kg, between about 700 μg/kg and about 1,000 μg/kg, between about 750 μg/kg and about 1,000 μg/kg, between about 800 μg/kg and about 1,000 μg/kg, between about 850 μg/kg and about 1,000 μg/kg, between about 900 μg/kg and about 1,000 μg/kg, or between about 950 μg/kg and about 1,000 μg/kg.

In some aspects, the IL-7 protein is administered at a dose of between about 700 μg/kg and about 900 μg/kg, between about 750 μg/kg and about 950 μg/kg, between about 700 μg/kg and about 850 μg/kg, between about 750 μg/kg and about 850 μg/kg, between about 700 μg/kg and about 800 μg/kg, between about 800 μg/kg and about 900 μg/kg, between about 750 μg/kg and about 850 μg/kg, or between about 850 μg/kg and about 950 μg/kg.

In some aspects, the IL-7 protein is administered at a dose of about 650 μg/kg, about 680 μg/kg, about 700 μg/kg, about 720 μg/kg, about 740 μg/kg, about 750 μg/kg, about 760 μg/kg, about 780 μg/kg, about 800 μg/kg, about 820 μg/kg, about 840 μg/kg, about 850 μg/kg, about 860 μg/kg, about 880 μg/kg, about 900 μg/kg, about 920 μg/kg, about 940 μg/kg, about 950 μg/kg, about 960 μg/kg, about 980 μg/kg, about 1,000 μg/kg, about 1,100 μg/kg, about 1200 μg/kg, about 1,300 μg/kg, about 1,400 μg/kg, about 1,440 μg/kg, about 1,500 μg/kg, about 1,600 μg/kg, about 1,700 μg/kg, about 1,800 μg/kg, about 1,900 μg/kg, or about 2,000 μg/kg. In certain aspects, the IL-7 is administered at a dosing frequency of about once a week, about once in two weeks, about once in three weeks, about once in four weeks, about once in five weeks, about once in six weeks, about once in seven weeks, about once in eight weeks, about once in nine weeks, about once in 10 weeks, about once in 11 weeks, or about once in 12 weeks.

In any of the methods disclosed here, in some aspects, the nucleotide vaccine comprises a DNA vaccine, mRNA vaccine, or both. In certain aspects, the nucleotide vaccine is a DNA vaccine. In some aspects, the IL-7 is administered as a protein (IL-7 protein), nucleic acid encoding the IL-7 protein, or both.

In some aspects, the subject is a human.

In some aspects, the IL-7 protein of a method disclosed herein is not a wild-type IL-7. In certain aspects, the IL-7 protein is a fusion protein. In certain aspects, the IL-7 protein comprises an oligopeptide consisting of 1 to 10 amino acid residues. In some aspects, oligopeptide comprises methionine (M), glycine (G), methionine-methionine (MM), glycine-glycine (GG), methionine-glycine (MG), glycine-methionine (GM), methionine-methionine-methionine (MMM), methionine-methionine-glycine (MMG), methionine-glycine-methionine (MGM), glycine-methionine-methionine (GMM), methionine-glycine-glycine (MGG), glycine-methionine-glycine (GMG), glycine-glycine-methionine (GGM), glycine-glycine-glycine (GGG), methionine-glycine-glycine-methionine (MGGM) (SEQ ID NO: 41), methionine-methionine-glycine-glycine (MMGG) (SEQ ID NO: 42), glycine-glycine-methionine-methionine (GGMM) (SEQ ID NO: 43), methionine-glycine-methionine-glycine (MGMG) (SEQ ID NO: 44), glycine-methionine-methionine-glycine (GMMG) (SEQ ID NO: 45), glycine-glycine-glycine-methionine (GGGM) (SEQ ID NO: 46), methionine-glycine-glycine-glycine (MGGG) (SEQ ID NO: 47), glycine-methionine-glycine-glycine (GMGG) (SEQ ID NO: 48), glycine-glycine-methionine-glycine (GGMG) (SEQ ID NO: 49), glycine-glycine-methionine-methionine-methionine (GGMMM) (SEQ ID NO: 50), glycine-glycine-glycine-methionine-methionine (GGGMM) (SEQ ID NO: 51), glycine-glycine-glycine-glycine-methionine (GGGGM) (SEQ ID NO: 52), methionine-glycine-methionine-methionine-methionine (MGMMM) (SEQ ID NO: 53), methionine-glycine-glycine-methionine-methionine (MGGMM) (SEQ ID NO: 54), methionine-glycine-glycine-glycine-methionine (MGGGM) (SEQ ID NO: 55), methionine-methionine-glycine-methionine-methionine (MMGMM) (SEQ ID NO: 56), methionine-methionine-glycine-glycine-methionine (MMGGM) (SEQ ID NO: 57), methionine-methionine-glycine-glycine-glycine (MMGGG) (SEQ ID NO: 58), methionine-methionine-methionine-glycine-methionine (MMMGM) (SEQ ID NO: 59), methionine-glycine-methionine-glycine-methionine (MGMGM) (SEQ ID NO: 60), glycine-methionine-glycine-methionine-glycine (GMGMG) (SEQ ID NO: 61), glycine-methionine-methionine-methionine-glycine (GMMMG) (SEQ ID NO: 62), glycine-glycine-methionine-glycine-methionine (GGMGM) (SEQ ID NO: 63), glycine-glycine-methionine-methionine-glycine (GGMMG) (SEQ ID NO: 64), glycine-methionine-methionine-glycine-methionine (GMMGM) (SEQ ID NO: 65), methionine-glycine-methionine-methionine-glycine (MGMMG) (SEQ ID NO: 66), glycine-methionine-glycine-glycine-methionine (GMGGM) (SEQ ID NO: 67), methionine-methionine-glycine-methionine-glycine (MMGMG) (SEQ ID NO: 68), glycine-methionine-methionine-glycine-glycine (GMMGG) (SEQ ID NO: 69), glycine-methionine-glycine-glycine-glycine (GMGGG) (SEQ ID NO: 70), glycine-glycine-methionine-glycine-glycine (GGMGG) (SEQ ID NO: 71), glycine-glycine-glycine-glycine-glycine (GGGGG) (SEQ ID NO: 72), or combinations thereof. In certain aspects, the oligopeptide is methionine-glycine-methionine (MGM).

In some aspects, the IL-7 protein comprises a half-life extending moiety. In certain aspects, the half-life extending moiety comprises an Fc, albumin, an albumin-binding polypeptide, Pro/Ala/Ser (PAS), a C-terminal peptide (CTP) of the R subunit of human chorionic gonadotropin, polyethylene glycol (PEG), long unstructured hydrophilic sequences of amino acids (XTEN), hydroxyethyl starch (HES), an albumin-binding small molecule, or a combination thereof. In some aspects, the half-life extending moiety is an Fc. In some aspects, the Fc is a hybrid Fc, comprising a hinge region, a CH2 domain, and a CH3 domain, wherein the hinge region comprises a human IgD hinge region, wherein the CH2 domain comprises a part of human IgD CH2 domain and a part of human IgG4 CH2 domain, and wherein the CH3 domain comprises a part of human IgG4 CH3 domain.

In some aspects, the IL-7 protein comprises an amino acid sequence having a sequence identity of at least about 70%, at least about 75%, at least about 80%, at least about 85%, at least about 90%, at least about 95%, at least about 96%, at least about 97%, at least about 98%, at least about 99%, or about 100% to SEQ ID NOs: 1-6 and 15-25.

In some aspects, the IL-7 is administered to the subject parenthetically, intramuscularly, subcutaneously, ophthalmic, intravenously, intraperitoneally, intradermally, intraorbitally, intracerebrally, intracranially, intraspinally, intraventricular, intrathecally, intracistemally, intracapsularly, intratumorally, or any combination thereof. In some aspects, the nucleotide vaccine is administered to the subject parenthetically, intramuscularly, cutaneously, subcutaneously, ophthalmic, intravenously, intraperitoneally, intradermally, intraorbitally, intracerebrally, intracranially, intraspinally, intraventricular, intrathecally, intracistemally, intracapsularly, intratumorally, or any combination thereof.

In some aspects, methods provided herein further comprises administering at least one additional therapeutic agent to the subject.

In any of the methods provided herein, in some aspects, the tumor antigen comprises guanylate cyclase C (GC-C), epidermal growth factor receptor (EGFR or erbB-1), human epidermal growth factor receptor 2 (HER2 or erbf12), erbB-3, erbB-4, MUC-1, melanoma-associated chondroitin sulfate proteoglycan (MCSP), mesothelin (MSLN), folate receptor 1 (FOLR1), CD4, CD19, CD20, CD22, CD30, CD33, CD38, CD44, CD44v6, CD44v7/8, CD70, CD123, CD138, CD171, CEA, CSPG4, CXCR5, c-Met, HERV-envelope protein, eriostin, Bigh3, SPARC, BCR, CD79, CD37, EGFRvIII, EGP2, EGP40, IGFr, L1CAM, AXL, Tissue Factor (TF), CD74, EpCAM, EphA2, MRP3cadherin 19 (CDH19), epidermal growth factor 2 (HER2), 5T4, 8H9, αᵥβ₆ integrin, BCMA, B7-H3, B7-H6, CAIX, CA9, FAP, FBP, fetal AchR, FRcc, GD2, GD3, Glypican-1 (GPC1), Glypican-2 (GPC2), Glypican-3 (GPC3), HLA-A1+MAGE1, HLA-A1+NY-ESO-1, IL-13Rcc2, Lewis-Y, KDR, MCSP, Mesothelin, Muc1, Muc16, NCAM, NKG2D ligands, NY-ESO-1, PRAME, PSC1, PSCA, PSMA, ROR1, ROR2, SP17, surviving, TAG72, TEMs, carcinoembryonic antigen, HMW-MAA, VEGF, CLDN18.2, neoantigen, or combinations thereof. In certain aspects, the tumor antigen is derived from a cancer comprising a breast cancer, head and neck cancer, uterine cancer, brain cancer, skin cancer, renal cancer, lung cancer, colorectal cancer, prostate cancer, liver cancer, bladder cancer, kidney cancer, pancreatic cancer, thyroid cancer, esophageal cancer, eye cancer, stomach (gastric) cancer, gastrointestinal cancer, ovarian cancer, carcinoma, sarcoma, leukemia, lymphoma, myeloma, or a combination thereof.

### BRIEF DESCRIPTION OF THE FIGURES

FIGS. 1A, 1B, 1C, 1D, 1E, and 1F show the effect of IL-7 administration at different time points after DNA vaccine administration. FIG. 1A provides a schematic of the experimental design. As shown, animals received three immunizations of the DNA vaccine at a dosing frequency of once every three days. The IL-7 was administered to the animals at either day 4 or day 13 post initial DNA vaccine administration. The peak tumor-specific T cell immune response was observed at day 11 post initial DNA vaccine administration. FIG. 1B provides images comparing the spleen size at day 11 in animals that received (i) the control vector or DNA vaccine alone (left picture) or (ii) DNA vaccine+IL-7 at day 4 post initial DNA administration (right picture). FIGS. 1C and 1D provide comparison of the frequency (# of IFN-γ-producing T cells/10⁶ total splenocytes) and total number per spleen of tumor-specific T cells (i.e., specific to one of the following epitopes: Lrrc27, Plekho1, or Pttg1) at day 11 post initial DNA vaccine administration from the different treatment groups. For each of the peptide stimulations (x-axis), the groups shown are as follows (from left to right) (i) control vector alone (G1); (ii) DNA vaccine alone (G2); and (iii) DNA vaccine+IL-7 at day 4 post initial DNA administration (G3). FIGS. 1E and 1F provide comparison of the frequency (# of IFN-γ-producing T cells/10⁶ total splenocytes) and total number of tumor-specific T cells (i.e., specific to one of the following epitopes: Lrrc27, Plekho1, or Pttg1) at day 20 post initial DNA vaccine administration from the following treatment groups: (i) control vector alone (G1); (ii) DNA vaccine alone (G2); (iii) DNA vaccine+IL-7 at day 4 post initial DNA administration (G3); and (iv) DNA vaccine+IL-7 at day 13 post initial DNA administration (G4). For each of the peptide stimulations (x-axis), the groups shown are as follows (from left to right) (i) control vector alone (G1); (ii) DNA vaccine alone (G2); (iii) DNA vaccine+IL-7 at day 4 post initial DNA administration (G3); and (iv) DNA vaccine+IL-7 at day 13 post initial DNA administration (G4).

FIGS. 2A, 2B, and 2C show the effect of IL-7 dosage on tumor-specific T cell immune response after DNA vaccine administration. FIG. 2A provides a schematic of the experimental design. As shown, animals received three immunizations of the DNA vaccine at a dosing frequency of once every three days. The IL-7 was administered to the animals at day 13 post initial DNA vaccine administration at one of the following doses: 5, 10, or 15 mg/kg. FIGS. 2B and 2C provide comparison of the frequency (# of IFN-γ-producing T cells/10⁶ total splenocytes) of tumor-specific T cells (i.e., specific to one of the following epitopes: Lrrc27, Plekho1, or Pttg1) at day 20 post initial DNA administration in the spleen and lymph nodes, respectively. For each of the peptide stimulations (x-axis), the groups shown are as follows (from left to right) (i) control vector alone (G1); (ii) DNA vaccine alone (G2); (iii) DNA vaccine+5 mg/kg of IL-7 (G3); (iv) DNA vaccine+10 mg/kg of IL-7 (G4); and (v) DNA vaccine+15 mg/kg of IL-7 (G5).

FIGS. 3A and 3B show the anti-tumor effects of a nucleotide vaccine and IL-7 combination therapy disclosed herein. FIG. 3A provides a schematic of the experimental design. As shown, animals received three immunizations of the DNA vaccine at a dosing frequency of once every three days. At day 8 post initial DNA immunization, animals were implanted subcutaneously with E0771 tumor cells (5×10⁵ cells/mouse). Peak tumor-specific T cell immune response was observed at about day 10 post initial DNA immunization. The IL-7 was administered to the animals at day 13 post initial DNA vaccine administration. FIG. 3B provides a comparison of the tumor volume in animals from the different treatment groups at various time points post initial DNA vaccine administration. The groups shown include: (i) control vector only (circle); (ii) DNA vaccine only (square); and (iii) DNA vaccine+IL-7 (triangle).

FIGS. 4A and 4B show the effect of a nucleotide vaccine and IL-7 combination therapy described herein on T cell-mediated cytotoxicity as measured using an in vivo CTL assay. FIG. 4A provides a schematic of the experimental design. FIG. 4B provides a comparison of the percent killing of the neoantigen-pulsed splenocytes in animals from the different treatment groups at days 22, 34, 41, and 51 post initial immunization. As described in Example 5, the different treatment groups included: (i) control vector only (“vector”); (ii) DNA vaccine only (“nAg”); (iii) IL-7 protein alone (“IL-7”); and (iv) DNA vaccine+IL-7 protein (“nAg+IL-7”). Percent killing of the pulsed splenocytes is shown normalized to the un-pulsed splenocyte control.

FIGS. 5A and 5B show the anti-tumor effects of a nucleotide vaccine and IL-7 combination therapy when administered after the occurrence of a tumor. FIG. 5A provides a schematic of the experimental design. FIG. 5B provides comparison of the frequency (# of IFN-γ-producing T cells/10⁶ total splenocytes) of tumor-specific T cells (i.e., specific to one of the following: Lrrc27, Plekho1, Pttg1, no peptide (“media”)—left to right in each of the treatment groups shown) at days 20 post animal randomization. The different treatment groups are shown along the x-axis and further described in Example 6.

FIGS. 6A and 6B show the anti-tumor effects of a nucleotide vaccine and IL-7 combination therapy as a prophylactic vaccine. FIG. 6A provides a schematic of the experimental design. FIG. 6B provides a comparison of tumor volume in animals from the different treatment groups at various time points post tumor inoculation.

### DETAILED DESCRIPTION OF THE DISCLOSURE

### I. Definitions

In order that the present disclosure can be more readily understood, certain terms are first defined. As used in this application, except as otherwise expressly provided herein, each of the following terms shall have the meaning set forth below. Additional definitions are set forth throughout the application.

Throughout this disclosure, the term “a” or “an” entity refers to one or more of that entity; for example, “an antibody,” is understood to represent one or more antibodies. As such, the terms “a” (or “an”), “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Système International de Unites (SI) accepted form. Numeric ranges are inclusive of the numbers defining the range. Unless otherwise indicated, amino acid sequences are written left to right in amino to carboxy orientation. The headings provided herein are not limitations of the various aspects of the disclosure, which can be had by reference to the specification as a whole. Accordingly, the terms defined immediately below are more fully defined by reference to the specification in its entirety.

The term “about” is used herein to mean approximately, roughly, around, or in the regions of. When the term “about” is used in conjunction with a numerical range, it modifies that range by extending the boundaries above and below the numerical values set forth. In general, the term “about” can modify a numerical value above and below the stated value by a variance of, e.g., 10 percent, up or down (higher or lower).

As used herein, “administering” refers to the physical introduction of a therapeutic agent or a composition comprising a therapeutic agent to a subject, using any of the various methods and delivery systems known to those skilled in the art. The different routes of administration for a therapeutic agent described herein include intravenous, intraperitoneal, intramuscular, cutaneous, subcutaneous, spinal, intratumorally, or other parenteral routes of administration, for example by injection or infusion. The phrase “parenteral administration” as used herein means modes of administration other than enteral and topical administration, usually by injection, and includes, without limitation, intravenous, intraperitoneal, intramuscular, intraarterial, intrathecal, intralymphatic, intralesional, intracapsular, intraorbital, intracardiac, intradermal, transtracheal, intratracheal, pulmonary, subcuticular, intraarticular, subcapsular, subarachnoid, intraventricle, intravitreal, epidural, and intrasternal injection and infusion, as well as in vivo electroporation. Alternatively, a therapeutic agent described herein can be administered via a non-parenteral route, such as a topical, epidermal, or mucosal route of administration, for example, intranasally, orally, vaginally, rectally, sublingually, or topically. Administering can also be performed, for example, once, a plurality of times, and/or over one or more extended periods.

As used herein, the term “antigen” refers to any natural or synthetic immunogenic substance, such as a protein, peptide, or hapten. In certain aspects, the antigen comprises a tumor antigen. The term “tumor antigen” refers to an antigen that is uniquely or differentially expressed on a tumor cell compared to normal healthy cells.

As used herein, the term “epitope” refers to a set of amino acid residues that is involved in recognition by a particular immunoglobulin, or in the context of T cells, those residues necessary or recognition by T cell receptor proteins and/or major histocompatibility complex (MHC) receptors (e.g., site on a tumor antigen to which a tumor-specific T cell can recognize and target). In an immune system setting, in vitro or in vivo, an epitope is the collective features of a molecule, such as primary, secondary and tertiary peptide structure, and charge, that together form a site recognized by an immunoglobulin, T cell receptor, or HLA molecule. Epitopes can be formed both from contiguous amino acids (linear epitope) or noncontiguous amino acids juxtaposed by tertiary folding of a protein (conformational epitopes). Epitopes formed from contiguous amino acids are typically retained on exposure to denaturing solvents, whereas epitopes formed by tertiary folding are typically lost on treatment with denaturing solvents. An epitope typically includes at least 3, and more usually, at least 5 or 8-10 amino acids in a unique spatial conformation. Methods of determining spatial conformation of epitopes include, for example, x-ray crystallography and 2-dimensional nuclear magnetic resonance. See, e.g., Epitope Mapping Protocols in Methods in Molecular Biology, Vol. 66, Glenn E. Morris, Ed (1996), which is incorporated herein by reference in its entirety.

As described herein, the term epitope can comprise both dominant and subdominant epitopes. As used herein, the term “dominant epitope” refers to an epitope (e.g., of a tumor antigen) that evokes a strong immune response. As used herein, the term “subdominant epitope” refers to an epitope (e.g., of a tumor antigen) that evokes a weak or no immune response.

As used herein, the term “vaccine” refers to an agent that is capable of inducing an immune in a subject upon administration. In some aspects, the vaccine is a “preventive” vaccine, which is administered to a subject not afflicted with a disease or disorder disclosed herein (e.g., cancer). Such vaccines are also referred to herein as “prophylactic” vaccines. In some aspects, the vaccine is a therapeutic vaccine. As used herein, the term “therapeutic” vaccine refers to a vaccine that is administered to a subject to treat a disease or disorder (e.g., prevent or reduce one or more symptoms associated with the disease or disorder).

As used herein, the terms “nucleotide vaccine,” “nucleic acid vaccine,” “nucleic acid-based vaccine,” and “genetic vaccine” can be used interchangeably and refer to a vaccine in which the antigenic component comprises a nucleic acid. Such vaccines are capable of delivering genetic materials encoding the antigen of interest (e.g., tumor antigen) into host cells, which subsequently produce the antigen and thereby, initiate an immune response that is capable of protecting the host against the disease or disorder from which the antigen was derived. In some aspects, a nucleotide vaccine comprises both DNA vaccine and RNA (e.g., mRNA) vaccine. In certain aspects, a nucleotide vaccine is a DNA vaccine (i.e., the antigenic component is a DNA sequence). In certain aspects, a nucleotide vaccine is a RNA (mRNA) vaccine (i.e., the antigenic component is a RNA sequence).

The term “naturally-occurring” as used herein as applied to an object refers to the fact that an object can be found in nature. For example, a polypeptide or polynucleotide sequence that is present in an organism (including viruses) that can be isolated from a source in nature and which has not been intentionally modified by man in the laboratory is naturally-occurring.

A “polypeptide” refers to a chain comprising at least two consecutively linked amino acid residues, with no upper limit on the length of the chain. One or more amino acid residues in the protein can contain a modification such as, but not limited to, glycosylation, phosphorylation or disulfide bond formation. A “protein” can comprise one or more polypeptides. Unless otherwise specified, the terms “protein” and “polypeptide” can be used interchangeably.

The term “nucleic acid molecule,” as used herein, is intended to include DNA molecules and RNA molecules. A nucleic acid molecule can be single-stranded or double-stranded, and can be cDNA.

The nucleic acids can be present in whole cells, in a cell lysate, or in a partially purified or substantially pure form. A nucleic acid is “isolated” or “rendered substantially pure” when purified away from other cellular components or other contaminants, e.g., other cellular nucleic acids (e.g., the other parts of the chromosome) or proteins, by standard techniques, including alkaline/SDS treatment, CsCl banding, column chromatography, agarose gel electrophoresis and others well known in the art. See, F. Ausubel, et al., ed. Current Protocols in Molecular Biology, Greene Publishing and Wiley Interscience, New York (1987).

Nucleic acids, e.g., cDNA, can be mutated, in accordance with standard techniques to provide gene sequences. For coding sequences, these mutations, can affect amino acid sequence as desired. In particular, DNA sequences substantially homologous to or derived from native V, D, J, constant, switches and other such sequences described herein are contemplated (where “derived” indicates that a sequence is identical or modified from another sequence).

“Conservative amino acid substitutions” refer to substitutions of an amino acid residue with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). In certain aspects, a predicted nonessential amino acid residue in an antibody is replaced with another amino acid residue from the same side chain family. Methods of identifying nucleotide and amino acid conservative substitutions which do not eliminate antigen binding are well-known in the art (see, e.g., Brummell et al., Biochem. 32: 1180-1187 (1993); Kobayashi et al. Protein Eng. 12(10):879-884 (1999); and Burks et al. Proc. Natl. Acad. Sci. USA 94:412-417 (1997)).

For nucleic acids, the term “substantial homology” indicates that two nucleic acids, or designated sequences thereof, when optimally aligned and compared, are identical, with appropriate nucleotide insertions or deletions, in at least about 80% of the nucleotides, at least about 90% to 95%, or at least about 98% to 99.5% of the nucleotides. Alternatively, substantial homology exists when the segments will hybridize under selective hybridization conditions, to the complement of the strand.

For polypeptides, the term “substantial homology” indicates that two polypeptides, or designated sequences thereof, when optimally aligned and compared, are identical, with appropriate amino acid insertions or deletions, in at least about 80% of the amino acids, at least about 90% to 95%, or at least about 98% to 99.5% of the amino acids.

The percent identity between two sequences is a function of the number of identical positions shared by the sequences (i.e., % homology=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm, e.g., as described in the non-limiting examples below.

The percent identity between two nucleotide sequences can be determined using the GAP program in the GCG software package (available at worldwideweb.gcg.com), using a NWSgapdna.CMP matrix and a gap weight of 40, 50, 60, 70, or 80 and a length weight of 1, 2, 3, 4, 5, or 6. The percent identity between two nucleotide or amino acid sequences can also be determined using the algorithm of E. Meyers and W. Miller (CABIOS, 4: 11-17 (1989)) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol. Biol. (48):444-453 (1970)) algorithm which has been incorporated into the GAP program in the GCG software package (available at worldwideweb.gcg.com), using either a Blossum 62 matrix or a PAM250 matrix, and a gap weight of 16, 14, 12, 10, 8, 6, or 4 and a length weight of 1, 2, 3, 4, 5, or 6.

The nucleic acid and protein sequences described herein can further be used as a “query sequence” to perform a search against public databases to, for example, identify related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to the nucleic acid molecules described herein. BLAST protein searches can be performed with the XBLAST program, score=50, wordlength=3 to obtain amino acid sequences homologous to the protein molecules described herein. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used. See worldwideweb.ncbi.nlm.nih.gov.

As used herein, the term “effector function” refers to a specialized function of a differentiated immune cell. An effector function of a T cell, for example, can be cytolytic activity or helper activity including the secretion of cytokines. An effector function in a naive, memory, or memory-type T cell can also include antigen-dependent proliferation.

The term “immune cell,” as used herein, refers to cells that play a role in the immune response. Accordingly, in some aspects, immune cells useful for the present disclosure are those cells that can play a role in the treatment and/or eradication of a solid tumor (e.g., possess anti-tumor activity). In some aspects, the immune cells comprise lymphocytes, neutrophils, monocytes, macrophages, dendritic cells, or any combination thereof. In certain aspects, the lymphocytes comprise T cells, tumor-infiltrating lymphocytes (TIL), lymphokine-activated killer cells, natural killer T (NKT) cells, or any combination thereof. In some aspects, the lymphocytes are T cells. In some aspects, the lymphocytes are NKT cells (e.g., invariant NKT cells).

The term “vector,” as used herein, is intended to refer to a nucleic acid molecule capable of transporting another nucleic acid to which it has been linked. One type of vector is a “plasmid,” which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”) In general, expression vectors of utility in recombinant DNA techniques are often in the form of plasmids. In the present specification, “plasmid” and “vector” can be used interchangeably as the plasmid is the most commonly used form of vector. However, also included are other forms of expression vectors, such as viral vectors (e.g., replication defective retroviruses, adenoviruses and adeno-associated viruses), which serve equivalent functions.

The term “recombinant host cell” (or simply “host cell”), as used herein, is intended to refer to a cell that comprises a nucleic acid that is not naturally present in the cell, and can be a cell into which a recombinant expression vector has been introduced. It should be understood that such terms are intended to refer not only to the particular subject cell but to the progeny of such a cell. Because certain modifications can occur in succeeding generations due to either mutation or environmental influences, such progeny cannot, in fact, be identical to the parent cell, but are still included within the scope of the term “host cell” as used herein.

A “cancer” refers a broad group of various diseases characterized by the uncontrolled growth of abnormal cells (or tumors) in the body. Unregulated cell division and growth results in the formation of malignant tumors that invade neighboring tissues and can also metastasize to distant parts of the body through the lymphatic system or bloodstream. Cancers that can be treated with the present disclosure include those associated with a solid tumor. Unless indicated otherwise, the terms “cancers” and “tumors” can be used interchangeably.

The term “fusion protein” refers to proteins created through the joining of two or more genes that originally coded for separate proteins. Translation of this fusion gene results in a single polypeptide or multiple polypeptides with functional properties derived from each of the original proteins. In some aspects, the two or more genes can comprise a substitution, a deletion, and/or an addition in its nucleotide sequence.

An “Fc receptor” or “FcR” is a receptor that binds to the Fc region of an immunoglobulin. FcRs that bind to an IgG antibody comprise receptors of the FcγR family, including allelic variants and alternatively spliced forms of these receptors. The FcγR family consists of three activating (FcγRI, FcγRIII, and FcγRIV in mice; FcγRIA, FcγRIIA, and FcγRIIIA in humans) and one inhibitory (FcγRIIB) receptor. Various properties of human FcγRs are known in the art. The majority of innate effector cell types coexpress one or more activating FcγR and the inhibitory FcγRIIB, whereas natural killer (NK) cells selectively express one activating Fc receptor (FcγRIII in mice and FcγRIIIA in humans) but not the inhibitory FcγRIIB in mice and humans. Human IgG1 binds to most human Fc receptors and is considered equivalent to murine IgG2a with respect to the types of activating Fc receptors that it binds to.

An “Fc region” (fragment crystallizable region) or “Fc domain” or “Fc” refers to the C-terminal region of the heavy chain of an antibody that mediates the binding of the immunoglobulin to host tissues or factors, including binding to Fc receptors located on various cells of the immune system (e.g., effector cells) or to the first component (C1q) of the classical complement system. Thus, an Fc region comprises the constant region of an antibody excluding the first constant region immunoglobulin domain (e.g., CH1 or CL). In IgG, IgA and IgD antibody isotypes, the Fc region comprises two identical protein fragments, derived from the second (CH2) and third (CH3) constant domains of the antibody's two heavy chains; IgM and IgE Fc regions comprise three heavy chain constant domains (CH domains 2-4) in each polypeptide chain. For IgG, the Fc region comprises immunoglobulin domains CH2 and CH3 and the hinge between CH1 and CH2 domains. Although the definition of the boundaries of the Fc region of an immunoglobulin heavy chain might vary, as defined herein, the human IgG heavy chain Fc region is defined to stretch from an amino acid residue D221 for IgG1, V222 for IgG2, L221 for IgG3 and P224 for IgG4 to the carboxy-terminus of the heavy chain, wherein the numbering is according to the EU index as in Kabat. The CH2 domain of a human IgG Fc region extends from amino acid 237 to amino acid 340, and the CH3 domain is positioned on C-terminal side of a CH2 domain in an Fc region, i.e., it extends from amino acid 341 to amino acid 447 or 446 (if the C-terminal lysine residue is absent) or 445 (if the C-terminal glycine and lysine residues are absent) of an IgG. As used herein, the Fc region can be a native sequence Fc, including any allotypic variant, or a variant Fc (e.g., a non-naturally occurring Fc). Fc can also refer to this region in isolation or in the context of an Fc-comprising protein polypeptide such as a “binding protein comprising an Fc region,” also referred to as an “Fc fusion protein” (e.g., an antibody or immunoadhesion).

A “native sequence Fc region” or “native sequence Fc” comprises an amino acid sequence that is identical to the amino acid sequence of an Fc region found in nature. Native sequence human Fc regions include a native sequence human IgG1 Fc region; native sequence human IgG2 Fc region; native sequence human IgG3 Fc region; and native sequence human IgG4 Fc region as well as naturally occurring variants thereof. Native sequence Fc include the various allotypes of Fcs (see, e.g., Jefferis et al. (2009) mAbs 1: 1).

Additionally, an Fc (native or variant) of the present disclosure can be in the form of having native sugar chains, increased sugar chains, or decreased sugar chains compared to the native form, or can be in a deglycosylated form. The immunoglobulin Fc sugar chains can be modified by conventional methods such as a chemical method, an enzymatic method, and a genetic engineering method using a microorganism. The removal of sugar chains from an Fc fragment results in a sharp decrease in binding affinity to the C1q part of the first complement component C1, and a decrease or loss of ADCC or CDC, thereby not inducing any unnecessary immune responses in vivo. In this regard, an immunoglobulin Fc region in a deglycosylated or aglycosylated form can be more suitable to the object of the present disclosure as a drug carrier. As used herein, the term “deglycosylation” refers to an Fc region in which sugars are removed enzymatically from an Fc fragment. Additionally, the term “aglycosylation” means that an Fc fragment is produced in an unglycosylated form by a prokaryote, and preferably in E. coli.

As used herein, the term “immune response” refers to a biological response within a vertebrate against foreign agents, which response protects the organism against these agents and diseases caused by them. An immune response is mediated by the action of a cell of the immune system (e.g., a T lymphocyte, B lymphocyte, natural killer (NK) cell, macrophage, eosinophil, mast cell, dendritic cell or neutrophil) and soluble macromolecules produced by any of these cells or the liver (including antibodies, cytokines, and complement) that results in selective targeting, binding to, damage to, destruction of, and/or elimination from the vertebrate's body of invading pathogens, cells or tissues infected with pathogens, cancerous or other abnormal cells, or, in cases of autoimmunity or pathological inflammation, normal human cells or tissues. An immune reaction includes, e.g., activation or inhibition of a T cell, e.g., an effector T cell or a Th cell, such as a CD4⁺ or CD8⁺ T cell, or the inhibition of a Treg cell.

As described herein, in some aspects, an immune response (e.g., such as that induced by a nucleotide vaccine disclosed herein) comprises a T cell immune response. As used herein, the term “T cell immune response” refers to an immune response mediated by T cells (e.g., effector CD4⁺ and/or CD8⁺ T cells). A T cell immune response can be generally divided into three phases: (i) expansion, (ii) contraction, and (iii) maintenance. Kumar et al., Immunity 48(2): 202-213 (February 2018); and Blair et al., J Immunol 187: 2310-2321 (2011). During the expansion phase, naïve T cells that recognize their cognate antigen become activated, resulting in clonal expansion and acquisition of effector function (e.g., production of inflammatory cytokines and expression of effector molecules, such as granzyme and perforin). Following the expansion phase, approximately 90-95% of the activated T cells at the peak of the response undergo apoptosis (i.e., contraction phase). The surviving population of activated T cells eventually differentiate into memory T cells that provide long-lasting protection to the host (i.e., maintenance phase).

The term “immunotherapy” refers to the treatment of a subject afflicted with, or at risk of contracting or suffering a recurrence of, a disease by a method comprising inducing, enhancing, suppressing or otherwise modifying an immune response. “Treatment” or “therapy” of a subject refers to any type of intervention or process performed on, or the administration of an active agent to, the subject with the objective of reversing, alleviating, ameliorating, inhibiting, slowing down or preventing the onset, progression, development, severity or recurrence of a symptom, complication or condition, or biochemical indicia associated with a disease.

As used herein, the term “tumor infiltrating lymphocytes” or “TILs” refers to lymphocytes (e.g., effector T cells) that have migrated from the periphery (e.g., from the blood) into a tumor. In some aspects, the tumor infiltrating lymphocytes are CD4+ TILs. In some aspects, the tumor infiltrating lymphocytes are CD8+ TILs.

An increased ability to stimulate an immune response or the immune system, can result from an enhanced agonist activity of T cell costimulatory receptors and/or an enhanced antagonist activity of inhibitory receptors. An increased ability to stimulate an immune response or the immune system can be reflected by a fold increase of the EC50 or maximal level of activity in an assay that measures an immune response, e.g., an assay that measures changes in cytokine or chemokine release, cytolytic activity (determined directly on target cells or indirectly via detecting CD107a or granzymes) and proliferation. The ability to stimulate an immune response or the immune system activity can be enhanced by at least 10%, 30%, 50%, 75%, 2 fold, 3 fold, 5 fold or more.

As used herein, the term “interleukin-7” or “IL-7” refers to IL-7 polypeptides and derivatives and analogs thereof having substantial amino acid sequence identity to wild-type mature mammalian IL-7 proteins and substantially equivalent biological activity, e.g., in standard bioassays or assays of IL-7 receptor binding affinity. Additional disclosure relating to IL-7 proteins that can be used with the present disclosure are provided elsewhere herein.

A “variant” of an IL-7 protein is defined as an amino acid sequence that is altered by one or more amino acids. The variant can have “conservative” changes, wherein a substituted amino acid has similar structural or chemical properties, e.g., replacement of leucine with isoleucine. More rarely, a variant can have “nonconservative” changes, e.g., replacement of a glycine with a tryptophan. Similar minor variations can also include amino acid deletions or insertions, or both. Guidance in determining which and how many amino acid residues can be substituted, inserted or deleted without abolishing biological activity can be found using computer programs well known in the art, for example software for molecular modeling or for producing alignments. The variant IL-7 proteins included within the present disclosure include IL-7 proteins that retain IL-7 activity. IL-7 polypeptides which also include additions, substitutions or deletions are also included within the present disclosure as long as the proteins retain substantially equivalent biological IL-7 activity. For example, truncations of IL-7 which retain comparable biological activity as the full length form of the IL-7 protein are included within the present disclosure. In some aspects, variant IL-7 proteins also include polypeptides that have at least about 70%, at least about 75%, at least about 80%, at least about 85%, at least about 90%, at least about 91%, at least about 92%, at least about 93%, at least about 93%, at least about 95%, at least about 96%, at least about 97%, at least about 98%, at least about 99%, or more sequence identity with wild-type IL-7.

As used herein, the term “signal sequence,” or equivalently, “signal peptide,” refers to a fragment directing the secretion of a biologically active molecule drug and a fusion protein, and it is cut off after being translated in a host cell. The signal sequence as used herein is a polynucleotide encoding an amino acid sequence initiating the movement of the protein penetrating the endoplasmic reticulum (ER) membrane. Useful signal sequences include an antibody light chain signal sequence, e.g., antibody 14.18 (Gillies et al., J. Immunol. Meth 1989. 125:191-202), an antibody heavy chain signal sequence, e.g., MOPC141 an antibody heavy chain signal sequence (Sakano et al., Nature, 1980.286: 676-683), and other signal sequences know in the art (e.g., see Watson et al., Nucleic Acid Research, 1984.12:5145-5164). The characteristics of signal peptides are well known in the art, and the signal peptides conventionally having 16 to 30 amino acids, but they can include more or less number of amino acid residues. Conventional signal peptides consist of three regions of the basic N-terminal region, a central hydrophobic region, and a more polar C-terminal region.

A “subject” includes any human or nonhuman animal. The term “nonhuman animal” includes, but is not limited to, vertebrates such as nonhuman primates, sheep, dogs, and rodents such as mice, rats and guinea pigs. In some aspects, the subject is a human. The terms “subject” and “patient” are used interchangeably herein.

The term “therapeutically effective amount” or “therapeutically effective dosage” refers to an amount of an agent that provides the desired biological, therapeutic, and/or prophylactic result. That result can be reduction, amelioration, palliation, lessening, delaying, and/or alleviation of one or more of the signs, symptoms, or causes of a disease, or any other desired alteration of a biological system. In reference to solid tumors, an effective amount comprises an amount sufficient to cause a tumor to shrink and/or to decrease the growth rate of the tumor (such as to suppress tumor growth) or to prevent or delay other unwanted cell proliferation. In some aspects, an effective amount is an amount sufficient to delay tumor development. In some aspects, an effective amount is an amount sufficient to prevent or delay tumor recurrence. An effective amount can be administered in one or more administrations. The effective amount of the drug or composition can: (i) reduce the number of cancer cells; (ii) reduce tumor size; (iii) inhibit, retard, slow to some extent and can stop cancer cell infiltration into peripheral organs; (iv) inhibit (i.e., slow to some extent and can stop tumor metastasis; (v) inhibit tumor growth; (vi) prevent or delay occurrence and/or recurrence of tumor; and/or (vii) relieve to some extent one or more of the symptoms associated with the cancer. In some aspects, a “therapeutically effective amount” is the amount of nucleotide vaccine and/or IL-7 protein clinically proven to affect a significant decrease in cancer or slowing of progression (regression) of cancer, such as an advanced solid tumor. The ability of a therapeutic agent to promote disease regression can be evaluated using a variety of methods known to the skilled practitioner, such as in human subjects during clinical trials, in animal model systems predictive of efficacy in humans, or by assaying the activity of the agent in in vitro assays.

The terms “dosing frequency,” “dosing schedule,” and “dosing interval” are used interchangeably and refer to the number of times a therapeutic agent (e.g., a nucleotide vaccine and/or IL-7) is administered to a subject within a specific time period. Dosing frequency can be indicated as the number of doses per a given time, for example, once per day, once a week, or once in two weeks. As used herein, “dosing frequency” is applicable where a subject receives multiple (or repeated) administrations of a therapeutic agent.

The term “within” when used to describe an administration of a therapeutic agent described herein (e.g., a nucleotide vaccine and/or IL-7) means that the therapeutic agent is administered on or before the accompanying duration of time. For example, when an IL-7 protein is administered “within 7 days” after the administration of the nucleotide vaccine, it can mean any of the following: the IL-7 protein is administered 7 days after the nucleotide vaccine administration, the IL-7 protein is administered 6 days after the nucleotide vaccine administration, the IL-7 protein is administered 5 days after the nucleotide vaccine administration, the IL-7 protein is administered 4 days after the nucleotide vaccine administration, the IL-7 protein is administered 3 days after the nucleotide vaccine administration, the IL-7 protein is administered 2 days after the nucleotide vaccine administration, the IL-7 protein is administered 1 day after the nucleotide vaccine administration, the IL-7 protein is administered concurrently with the nucleotide vaccine administration, and any duration of time therein.

As used herein, the term “standard of care” refers to a treatment that is accepted by medical experts as a proper treatment for a certain type of disease and that is widely used by healthcare professionals. The term can be used interchangeable with any of the following terms: “best practice,” “standard medical care,” and “standard therapy.”

By way of example, an “anti-cancer agent” promotes cancer regression in a subject or prevents further tumor growth. In certain aspects, a therapeutically effective amount of the drug promotes cancer regression to the point of eliminating the cancer. “Promoting cancer regression” means that administering an effective amount of the drug, alone or in combination with an anti-neoplastic agent, results in a reduction in tumor growth or size, necrosis of the tumor, a decrease in severity of at least one disease symptom, an increase in frequency and duration of disease symptom-free periods, or a prevention of impairment or disability due to the disease affliction. In addition, the terms “effective” and “effectiveness” with regard to a treatment includes both pharmacological effectiveness and physiological safety. Pharmacological effectiveness refers to the ability of the drug to promote cancer regression in the patient. Physiological safety refers to the level of toxicity, or other adverse physiological effects at the cellular, organ and/or organism level (adverse effects) resulting from administration of the drug.

By way of example, for the treatment of tumors, a therapeutically effective amount of an anti-cancer agent can inhibit cell growth or tumor growth by at least about 10%, at least about 20%, by at least about 40%, by at least about 60%, or by at least about 80% relative to untreated subjects or, in certain aspects, relative to patients treated with a standard-of-care therapy. In some aspects, tumor regression can be observed and continue for a period of at least about 20 days, at least about 40 days, or at least about 60 days. Notwithstanding these ultimate measurements of therapeutic effectiveness, evaluation of immunotherapeutic drugs must also make allowance for “immune-related” response patterns.

As used herein, the term “immune checkpoint inhibitor” refers to molecules that totally or partially reduce, inhibit, interfere with or modulate one or more immune checkpoint proteins. Immune checkpoint proteins regulate T-cell activation or function. Numerous checkpoint proteins are known, such as CTLA-4 and its ligands CD80 and CD86; and PD-1 with its ligands PD-L1 and PD-L2. Pardoll, D. M., Nat Rev Cancer 12(4):252-64 (2012). These proteins are responsible for co-stimulatory or inhibitory interactions of T-cell responses. Immune checkpoint proteins regulate and maintain self-tolerance and the duration and amplitude of physiological immune responses. Immune checkpoint inhibitors include antibodies or are derived from antibodies.

As used herein, the terms “ug” and “uM” are used interchangeably with “μg” and “μM,” respectively.

Various aspects described herein are described in further detail in the following subsections.

### II. Methods of the Disclosure

Disclosed herein is a method for treating a tumor (or a cancer associated with a tumor) in a subject in need thereof, comprising administering to the subject a nucleotide vaccine in combination with an interleukin-7 (IL-7) protein. In some aspects, the nucleotide vaccine encodes a tumor antigen, such that the administration of the nucleotide vaccine is capable of inducing a tumor-specific T cell immune response in the subject. In some aspects, the IL-7 protein is administered to the subject after the peak expansion phase of the tumor-specific T cell immune response induced by the nucleotide vaccine. As used herein, the term “peak expansion phase” refers to the time point at which the number of tumor-specific T cells, e.g., induced by the nucleotide vaccine, is the greatest. In some aspects, the peak expansion phase marks the beginning of the contraction phase of a T cell immune response. As is apparent from the present disclosure, the peak expansion phase of the nucleotide vaccine-induced tumor-specific T cell immune response can differ depending on whether the nucleotide vaccine is administered therapeutically (i.e., after the occurrence of a tumor) or prophylactically (i.e., before the occurrence of a tumor). The peak expansion phase of a T cell immune response can be determined using any suitable methods known in the art (e.g., ELISPOT and flow cytometry).

For instance, when the nucleotide vaccine is administered prophylactically, in some aspects, the peak expansion of the nucleotide vaccine-induced tumor-specific T cell immune response occurs at about seven days, about eight days, about nine days, about ten days, about 11 days, about 12 days, about 13 days, about 14 days, or about 15 days after initial activation (i.e., antigen encounter) of the antigen-specific T cells (e.g., after initial administration of a nucleotide vaccine encoding a tumor antigen to the subject). In certain aspects, the peak expansion of the tumor-specific T cell immune response occurs at about 11 days after initial activation of the antigen-specific T cells (e.g., after initial administration of a nucleotide vaccine encoding a tumor antigen to the subject). In some aspects, when the nucleotide vaccine is administered therapeutically, the peak expansion can occur earlier. For instance, not to be bound by any one theory, in a therapeutic setting, tumor-specific T cells can already exist in the subject from an earlier encounter with the existing tumor. Accordingly, as will be apparent to those skilled in the art, when the nucleotide vaccines described herein are administered to such a subject, the existing tumor-specific T cells (e.g., memory T cells) could respond more quickly (compared to T cells that are seeing the antigen for the first time), resulting in a faster T cell kinetics (i.e., an earlier peak expansion).

In connection with the present disclosure, Applicant has discovered that the administration of an IL-7 protein after the peak expansion phase can improve a tumor-specific T cell immune response (e.g., induced by a nucleotide vaccine disclosed herein) compared to the corresponding value (i.e., tumor-specific T cell immune response) observed in a reference. As used herein, the term “reference” can refer to a corresponding individual that (i) only received the nucleotide vaccine alone, (ii) only received the IL-7 protein alone, (iii) received both the nucleotide vaccine and IL-7 protein, but the IL-7 protein was administered prior to the peak expansion phase, (iv) received neither the nucleotide vaccine nor the IL-7 protein, or (iv) combinations thereof.

Accordingly, as demonstrated herein, an IL-7 protein described herein is generally administered to the subject after the administration of the nucleotide vaccine. However, as also demonstrated herein, Applicant has further identified that administering an IL-7 protein at other time points (e.g., other than after the peak expansion phase of the tumor-specific T cell immune response) after nucleotide vaccine administration can also have therapeutic effects, e.g., particularly where the nucleotide vaccine is administered as a therapeutic vaccine. For instance, in some aspects, the IL-7 protein is administered to the subject within about six hours, within about 12 hours, within about one day, within about two days, within about three days, within about four days, within about five days, within about six days, within about one week, within about two weeks, without about three weeks, or within about four weeks after the administration of the nucleotide vaccine administration. In some aspects, the IL-7 protein is administered within about one week after the nucleotide vaccine administration. In some aspects, the IL-7 protein is administered within about five days after the nucleotide vaccine administration. In some aspects, the IL-7 protein is administered within about four days after the nucleotide vaccine administration. In some aspects, the IL-7 protein is administered within about three days after the nucleotide vaccine administration. In some aspects, the IL-7 protein is administered within about two days after the nucleotide vaccine administration. In some aspects, the IL-protein is administered within about one day after the nucleotide vaccine administration. In some aspects, the IL-7 protein is administered concurrently with the nucleotide vaccine administration.

In some aspects, an improved tumor-specific T cell immune response can result in reduced tumor growth in the subject. Accordingly, in some aspects, administering a nucleotide vaccine in combination with an IL-7 protein, wherein the IL-7 protein is administered after the peak expansion phase of the tumor-specific T cell immune response, can inhibit and/or reduce tumor growth (e.g., tumor volume or weight) in the subject compared to the reference. In certain aspects, the tumor growth is reduced by at least about 5%, at least about 10%, at least about 20%, at least about 30%, at least about 40%, at least about 50%, at least about 60%, at least about 70%, at least about 80%, at least about 90%, or about 100% after the administration compared to the corresponding value (i.e., tumor growth) in the reference. Similarly, in some aspects, administering an IL-7 protein (i) after the administration of the nucleotide vaccine or (ii) concurrently with the administration of the nucleotide vaccine can inhibit and/or reduce tumor growth (e.g., tumor volume or weight) in the subject compared to the reference. In certain aspects, the tumor growth is reduced by at least about 5%, at least about 10%, at least about 20%, at least about 30%, at least about 40%, at least about 50%, at least about 60%, at least about 70%, at least about 80%, at least about 90%, or about 100% after the administration compared to the corresponding value (i.e., tumor growth) in the reference.

In some aspects, an improved tumor-specific T cell immune response can result in a more prolonged tumor-specific T cell immune response in the subject. Not to be bound by any one theory, in certain aspects, the prolonged tumor-specific T cell immune response is due to increased survival (e.g., during the contraction phase) of the tumor-specific T cells. Accordingly, in some aspects, administering a nucleotide vaccine in combination with an IL-7 protein, wherein the IL-7 protein is administered after the peak expansion phase of the tumor-specific T cell immune response, can increase the survival of the tumor-specific T cells (e.g., during the contraction phase) in the subject compared to the corresponding value in the reference. In certain aspects, the survival of the tumor-specific T cells (e.g., during the contraction phase) is increased by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 35-fold, at least about 40-fold, at least about 45-fold, or at least about 50-fold or more, compared to the corresponding value in the reference. Similarly, in some aspects, administering an IL-7 protein (i) after the administration of the nucleotide vaccine or (ii) concurrently with the administration of the nucleotide vaccine can prolong the tumor-specific T cell immune response in the subject compared to the reference. In certain aspects, the tumor-specific T cell immune response is prolonged by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 35-fold, at least about 40-fold, at least about 45-fold, or at least about 50-fold or more, compared to the corresponding value in the reference.

In some aspects, the prolonged tumor-specific T cell immune response is due to increased resistance of the tumor-specific T cells to apoptosis (e.g., during the contraction phase of the immune response). In certain aspects, the resistance of the tumor-specific T cells to apoptosis (e.g., during the contraction phase) is increased by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 35-fold, at least about 40-fold, at least about 45-fold, or at least about 50-fold or more, compared to the corresponding value in the reference.

In some aspects, increasing the initial expansion of the tumor-specific T cells can also help prolong a tumor-specific T cell immune response (e.g., by increasing the overall number of tumor-specific T cells). Accordingly, in some aspects, administering an IL-7 protein (i) after the administration of the nucleotide vaccine or (ii) concurrently with the administration of the nucleotide vaccine can increase the initial expansion of the nucleotide vaccine-induced tumor-specific T cells by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 35-fold, at least about 40-fold, at least about 45-fold, or at least about 50-fold or more compared to the corresponding value in the reference.

In some aspects, an increased survival and/or increased resistance to apoptosis can increase the number of tumor-specific T cells in the subject compared to the corresponding value in the reference. In certain aspects, the number of tumor-specific T cells in the subject is increased by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 35-fold, at least about 40-fold, at least about 45-fold, or at least about 50-fold or more, compared to the corresponding value in the reference.

In some aspects, an improved tumor-specific T cell immune response comprises an increased cytotoxic activity of the tumor-specific T cells. For instance, in some aspects, administering a nucleotide vaccine in combination with an IL-7 protein increases the ability of the tumor-specific T cells to kill a cell expressing a tumor antigen (e.g., tumor cell). In some aspects, the cytotoxic activity of the tumor-specific T cells is increased by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 35-fold, at least about 40-fold, at least about 45-fold, or at least about 50-fold or more, compared to the corresponding value in the reference (e.g., corresponding subject that did not receive the combination treatment).

As described herein, an expansion phase of a T cell immune response is generally followed by a contraction phase, during which a large fraction (e.g., 90-95%) of the activated effector T cells undergo apoptosis with the surviving effector T cells differentiating into long-lived memory T cells. Accordingly, in some aspects, the increased survival and/or resistance of the tumor-specific T cells (e.g., during the contraction phase) to apoptosis can result in greater number of tumor-specific memory T cells in the subject. Similarly, in some aspects, increasing the initial expansion of the tumor-specific T cells can also help result in greater number of tumor-specific memory T cells in the subject. In some aspects, the number of tumor-specific memory T cells in the subject is increased by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 35-fold, at least about 40-fold, at least about 45-fold, or at least about 50-fold or more, compared to the corresponding value in the reference.

In some aspects, an improved tumor-specific T cell immune response comprises an expanded T cell receptor repertoire of a tumor-specific T cell immune response. For instance, in certain aspects, methods of the present disclosure (e.g., administering a nucleotide vaccine in combination with IL-7, wherein the IL-7 is administered to subject after the peak expansion phase of a tumor-specific T cell immune response) can increase the number of epitopes against which the tumor-specific T cell immune response is induced, compared to the corresponding value in the reference. Similarly, in some aspects administering an IL-7 protein (i) after the administration of the nucleotide vaccine or (ii) concurrently with the administration of the nucleotide vaccine can increase the number of epitopes against which the tumor-specific T cell immune response is induced, compared to the corresponding value in the reference.

In some aspects, the number of epitopes against which the tumor-specific T cell immune response is induced is increased by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 35-fold, at least about 40-fold, at least about 45-fold, or at least about 50-fold or more, compared to the reference.

In some aspects, tumor-specific T cells disclosed herein (i.e., induced after administration of a nucleotide vaccine encoding a tumor antigen in combination with IL-7, wherein the IL-7 is administered after the peak expansion phase of a tumor-specific T cell immune response) is capable of targeting one or more epitopes of a tumor antigen comprising Lrrc27, Plekho1, Pttg1, Xpo4, Exoc4, Pank3, Tmem101, Map3k6, Met, BC057079, Hist1h3e, Prkag1, Neil3, or combinations thereof. Similarly, in some aspects, tumor-specific T cells produced after the administration of an IL-7 protein (i) after the administration of the nucleotide vaccine or (ii) concurrently with the administration of the nucleotide vaccine can target one or more epitopes of a tumor antigen comprising Lrrc27, Plekho1, Pttg1, Xpo4, Exoc4, Pank3, Tmem101, Map3k6, Met, BC057079, Hist1h3e, Prkag1, Neil3, or combinations thereof. Additional disclosures regarding tumor antigens that can be targeted using the methods disclosed herein are provided else wherein the present disclosure. In certain aspects, tumor-specific T cells described herein can target one, two, three, four, five, six, seven, eight, nine, ten, 11, 12, or all of the tumor epitopes described above. In some aspects, the tumor-specific T cells of the present disclosure are capable of targeting the following epitopes: Lrrc27, Plekho1, and Pttg1.

Accordingly, in some aspects, the present disclosure is directed to a method of increasing a T cell immune response against a subdominant epitope of a tumor antigen in a subject in need thereof, comprising administering to the subject a nucleotide vaccine encoding a tumor antigen, which comprises the subdominant epitope, in combination with an IL-7, wherein the IL-7 is administered to the subject after the peak expansion phase of the tumor-specific T cell immune response. In some aspects, a method of increasing a T cell immune response against a subdominant epitope of a tumor antigen in a subject in need thereof, comprises administering to the subject a nucleotide vaccine encoding a tumor antigen, which comprises the subdominant epitope, in combination with an IL-7, wherein the IL-7 protein is administered: (i) after the administration of the nucleotide vaccine or (ii) concurrently with the administration of the nucleotide vaccine. In certain aspects, a T cell immune response against a subdominant epitope of a tumor antigen is increased by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 35-fold, at least about 40-fold, at least about 45-fold, or at least about 50-fold or more, compared to a corresponding value in a reference.

As described herein, in some aspects, methods of the present disclosure comprises administering a nucleotide vaccine encoding a tumor antigen in combination with IL-7, wherein the IL-7 is administered after the peak expansion phase of a tumor-specific T cell immune response. In some aspects, the IL-7 is administered to the subject (e.g., suffering from a tumor) at least about one day, about two days, about three days, about four days, about five days, about six days, about seven days, about eight days, about nine days, about 10 days, about 11 days, about 12 days, about 13 days, about 14 days, about 15 days, about 16 days, about 17 days, about 18 days, about 19 days, about 20 days, about 21 days, about 22 days, about 23 days, about 24 days, about 25 days, about 26 days, about 27 days, about 28 days, about 29 days, or about 30 days or more after the peak expansion phase of the tumor-specific T cell immune response. In certain aspects, the IL-7 is administered at about two days after the peak expansion phase of the tumor-specific T cell immune response.

Non-limiting examples of cancers (or tumors) that can be treated with methods disclosed herein include squamous cell carcinoma, small-cell lung cancer (SCLC), non-small cell lung cancer, squamous non-small cell lung cancer (NSCLC), nonsquamous NSCLC, gastrointestinal cancer, renal cancer (e.g., clear cell carcinoma), ovarian cancer, liver cancer (e.g., hepatocellular carcinoma), colorectal cancer, endometrial cancer, kidney cancer (e.g., renal cell carcinoma (RCC)), prostate cancer (e.g., hormone refractory prostate adenocarcinoma), thyroid cancer, pancreatic cancer, cervical cancer, stomach cancer, bladder cancer, hepatoma, breast cancer, colon carcinoma, and head and neck cancer (or carcinoma), gastric cancer, germ cell tumor, pediatric sarcoma, sinonasal natural killer, melanoma (e.g., metastatic malignant melanoma, such as cutaneous or intraocular malignant melanoma), bone cancer, skin cancer, uterine cancer, cancer of the anal region, testicular cancer, carcinoma of the fallopian tubes, carcinoma of the endometrium, carcinoma of the cervix, carcinoma of the vagina, carcinoma of the vulva, cancer of the esophagus (e.g., gastroesophageal junction cancer), cancer of the small intestine, cancer of the endocrine system, cancer of the parathyroid gland, cancer of the adrenal gland, sarcoma of soft tissue, cancer of the urethra, cancer of the penis, solid tumors of childhood, cancer of the ureter, carcinoma of the renal pelvis, tumor angiogenesis, pituitary adenoma, Kaposi's sarcoma, epidermoid cancer, squamous cell cancer, T-cell lymphoma, environmentally-induced cancers including those induced by asbestos, virus-related cancers or cancers of viral origin (e.g., human papilloma virus (HPV-related or -originating tumors)), and hematologic malignancies derived from either of the two major blood cell lineages, i.e., the myeloid cell line (which produces granulocytes, erythrocytes, thrombocytes, macrophages and mast cells) or lymphoid cell line (which produces B, T, NK and plasma cells), such as all types of leukemias, lymphomas, and myelomas, e.g., acute, chronic, lymphocytic and/or myelogenous leukemias, such as acute leukemia (ALL), acute myelogenous leukemia (AML), chronic lymphocytic leukemia (CLL), and chronic myelogenous leukemia (CML), undifferentiated AML (MO), myeloblastic leukemia (M1), myeloblastic leukemia (M2; with cell maturation), promyelocytic leukemia (M3 or M3 variant [M3V]), myelomonocytic leukemia (M4 or M4 variant with eosinophilia [M4E]), monocytic leukemia (M5), erythroleukemia (M6), megakaryoblastic leukemia (M7), isolated granulocytic sarcoma, and chloroma; lymphomas, such as Hodgkin's lymphoma (HL), non-Hodgkin's lymphoma (NHL), B cell hematologic malignancy, e.g., B-cell lymphomas, T-cell lymphomas, lymphoplasmacytoid lymphoma, monocytoid B-cell lymphoma, mucosa-associated lymphoid tissue (MALT) lymphoma, anaplastic (e.g., Kil+) large-cell lymphoma, adult T-cell lymphoma/leukemia, mantle cell lymphoma, angio immunoblastic T-cell lymphoma, angiocentric lymphoma, intestinal T-cell lymphoma, primary mediastinal B-cell lymphoma, precursor T-lymphoblastic lymphoma, T-lymphoblastic; and lymphoma/leukaemia (T-Lbly/T-ALL), peripheral T-cell lymphoma, lymphoblastic lymphoma, post-transplantation lymphoproliferative disorder, true histiocytic lymphoma, primary effusion lymphoma, B cell lymphoma, lymphoblastic lymphoma (LBL), hematopoietic tumors of lymphoid lineage, acute lymphoblastic leukemia, diffuse large B-cell lymphoma, Burkitt's lymphoma, follicular lymphoma, diffuse histiocytic lymphoma (DHL), immunoblastic large cell lymphoma, precursor B-lymphoblastic lymphoma, cutaneous T-cell lymphoma (CTLC) (also called mycosis fungoides or Sezary syndrome), and lymphoplasmacytoid lymphoma (LPL) with Waldenstrom's macroglobulinemia; myelomas, such as IgG myeloma, light chain myeloma, nonsecretory myeloma, smoldering myeloma (also called indolent myeloma), solitary plasmocytoma, and multiple myelomas, chronic lymphocytic leukemia (CLL), hairy cell lymphoma; hematopoietic tumors of myeloid lineage, tumors of mesenchymal origin, including fibrosarcoma and rhabdomyoscarcoma; seminoma, teratocarcinoma, tumors of mesenchymal origin, including fibrosarcoma, rhabdomyoscaroma, and osteosarcoma; and other tumors, including melanoma, xeroderma pigmentosum, keratoacanthoma, seminoma, thyroid follicular cancer and teratocarcinoma, hematopoietic tumors of lymphoid lineage, for example T-cell and B-cell tumors, including but not limited to T-cell disorders such as T-prolymphocytic leukemia (T-PLL), including of the small cell and cerebriform cell type; large granular lymphocyte leukemia (LGL) of the T-cell type; a/d T-NHL hepatosplenic lymphoma; peripheral/post-thymic T cell lymphoma (pleomorphic and immunoblastic subtypes); angiocentric (nasal) T-cell lymphoma; cancer of the head or neck, renal cancer, rectal cancer, cancer of the thyroid gland; acute myeloid lymphoma, and any combinations thereof.

In some aspects, a cancer (or tumor) that can be treated comprises a breast cancer, head and neck cancer, uterine cancer, brain cancer, skin cancer, renal cancer, lung cancer, colorectal cancer, prostate cancer, liver cancer, bladder cancer, kidney cancer, pancreatic cancer, thyroid cancer, esophageal cancer, eye cancer, stomach (gastric) cancer, gastrointestinal cancer, carcinoma, sarcoma, leukemia, lymphoma, myeloma, or a combination thereof. In certain aspects, a cancer (or tumor) that can be treated with the present methods is breast cancer. In some aspects, breast cancer is a triple negative breast cancer (TNBC). In some aspects, a cancer (or tumor) that can be treated is a brain cancer. In certain aspects, brain cancer is a glioblastoma. In some aspects, a cancer (or tumor) that can be treated with the present methods is skin cancer. In some aspects, skin cancer is a basal cell carcinoma (BCC), cutaneous squamous cell carcinoma (cSCC), melanoma, Merkel cell carcinoma (MCC), or a combination thereof. In certain aspects, a head and neck cancer is a head and neck squamous cell carcinoma. In further aspects, a lung cancer is a small cell lung cancer (SCLC). In some aspects, an esophageal cancer is gastroesophageal junction cancer. In certain aspects, a kidney cancer is renal cell carcinoma. In some aspects, a liver cancer is hepatocellular carcinoma.

In some aspects, the methods described herein (e.g., administering a nucleotide vaccine encoding a tumor antigen in combination with IL-7, wherein the IL-7 is administered after the peak expansion phase of a tumor-specific T cell immune response or wherein the IL-7 is administered: (i) after the administration of the nucleotide vaccine or (ii) concurrently with the administration of the nucleotide vaccine) can also be used for treatment of metastatic cancers, unresectable, refractory cancers (e.g., cancers refractory to previous cancer therapy, e.g., immunotherapy, e.g., with a blocking anti-PD-1 antibody), and/or recurrent cancers. In certain aspects, the previous cancer therapy comprises a chemotherapy. In some aspects, the chemotherapy comprises a platinum-based therapy. In some aspects, the platinum-based therapy comprises a platinum-based antineoplastic selected from the group consisting of cisplatin, carboplatin, oxaliplatin, nedaplatin, triplatin tetranitrate, phenanthriplatin, picoplatin, satraplatin, and any combination thereof. In certain aspects, the platinum-based therapy comprises cisplatin. In further aspects, the platinum-based therapy comprises carboplatin.

In some aspects, methods disclosed herein (e.g., administering a nucleotide vaccine encoding a tumor antigen in combination with IL-7, wherein the IL-7 is administered after the peak expansion phase of a tumor-specific T cell immune response or wherein the IL-7 is administered: (i) after the administration of the nucleotide vaccine or (ii) concurrently with the administration of the nucleotide vaccine) effectively increases the duration of survival of a subject in need thereof (e.g., afflicted with a tumor). For example, in some aspects, duration of survival of the subject is increased by at least about 1 month, at least about 2 months, at least about 3 months, at least about 4 months, at least about 5 months, at least about 6 months, at least about 7 months, at least about 8 months, at least about 9 months, at least about 10 months, at least about 11 months, or at least about 1 year or more when compared to a reference individual (e.g., corresponding subject treated with IL-7 protein alone or with a bispecific antibody alone). In other aspects, the methods disclosed herein increases duration of survival of the subject at a level higher than (about one month higher than, about two months higher than, about three months higher than, about four months higher than, about five months higher than, about six months higher than, about seven months higher than, about eight months higher than, about nine months higher than, about ten months higher than, about eleven months higher than, or about one year higher than) the duration of survival of a reference subject.

In some aspects, methods of the present disclosure (e.g., administering a nucleotide vaccine encoding a tumor antigen in combination with IL-7, wherein the IL-7 is administered after the peak expansion phase of a tumor-specific T cell immune response or wherein the IL-7 is administered: (i) after the administration of the nucleotide vaccine or (ii) concurrently with the administration of the nucleotide vaccine) effectively increase the duration of progression-free survival of a subject (e.g., cancer patient). For example, the progression free survival of the subject is increased by at least about 1 month, at least about 2 months, at least about 3 months, at least about 4 months, at least about 5 months, at least about 6 months, at least about 7 months, at least about 8 months, at least about 9 months, at least about 10 months, at least about 11 months, or at least about 1 year when compared to a reference subject.

In some aspects, methods disclosed herein (e.g., administering a nucleotide vaccine encoding a tumor antigen in combination with IL-7, wherein the IL-7 is administered after the peak expansion phase of a tumor-specific T cell immune response or wherein the IL-7 is administered: (i) after the administration of the nucleotide vaccine or (ii) concurrently with the administration of the nucleotide vaccine) effectively increases the response rate in a group of subjects. For example, the response rate in a group of subjects is increased by at least about 2%, at least about 3%, at least about 4%, at least about 5%, at least about 10%, at least about 15%, at least about 20%, at least about 25%, at least about 30%, at last about 35%, at least about 40%, at least about 45%, at least about 50%, at least about 55%, at least about 60%, at least about 70%, at least about 75%, at least about 80%, at least about 85%, at least about 90%, at least about 95%, at least about 99% or at least about 100% when compared to a reference subject.

As demonstrated herein, in some aspects, the combination therapy described herein (e.g., combination of a nucleotide vaccine and an IL-7 protein) can also be used as a prophylactic treatment against a disease or disorder described herein (e.g., cancer). Accordingly, in some aspects, provided herein is a method of preventing or reducing the occurrence of a tumor in a subject in need thereof, comprising administering to the subject a nucleotide vaccine encoding a tumor antigen in combination with an IL-7 protein, wherein the administration of the nucleotide vaccine induces a tumor-specific T cell immune response, and wherein the nucleotide vaccine, the IL-7 protein, or both the nucleotide vaccine and the IL-7 protein are administered to the subject prior to the occurrence of the tumor.

In some aspects, the nucleotide vaccine, the IL-7 protein, or both the nucleotide vaccine and the IL-7 protein are administered to the subject at least about one day, at least about two days, at least about three days, at least about four days, at least about five days, at least about six days, at least about seven days, at least about two weeks, at least about three weeks, at least about four weeks, at least about two months, at least about three months, at least about four months, at least about five months, at least about six months, at least about seven months, at least about eight months, at least about nine months, at least about 10 months, at least 11 months, or at least about one year prior to the occurrence of a tumor.

In some aspects, administering the nucleotide vaccine, the IL-7 protein, or both the nucleotide vaccine and the IL-7 protein as described above can help reduce the occurrence of the tumor by at least about 5%, at least about 10%, at least about 20%, at least about 30%, at least about 40%, at least about 50%, at least about 60%, at least about 70% at least about 80%, at least about 90%, or at least about 100%, compared to a reference (e.g., corresponding subject that did not receive the combination treatment as described above).

In some aspects, the subject being treated in the methods of the present disclosure is a nonhuman animal, such as a rat or a mouse. In some aspects, the subject being treated is a human.

In some aspects, IL-7 (e.g., such as those disclosed herein) is administered at a weight-based dose. In certain aspects, the IL-7 is administered at a dose between about 5 mg/kg and about 15 mg/kg. In some aspects, the IL-7 is administered at a dose of about 5 mg/kg.

In some aspects, a nucleotide vaccine described herein (e.g., encoding a tumor antigen and/or IL-7) can be administered at a dosage in the range of about 0.1 μg to about 200 mg. In certain aspects, the dosage is in the range of about 0.6 mg to about 100 mg. In further aspects, the dosage is in the range of about 1.2 mg to about 50 mg. In certain aspects, each dose of a nucleotide vaccine encoding a tumor antigen is about 4 μg. In some aspects, methods disclosed herein comprise administering a single dose of the nucleotide vaccine to a subject (e.g., suffering from a tumor). In some aspects, multiple does of the nucleotide vaccines are administered to the subject. In some of these aspects, the nucleotide vaccine is administered to the subject at a dosing frequency of about once a day, about once every two days, about once every three days, about once every four days, about once every five days, about once every six days, or about once every seven days. In some aspects, the nucleotide vaccine is administered about once every seven days (i.e., once a week). In some aspects, the nucleotide vaccine is administered about once a month. In certain aspects, the nucleotide vaccine is administered about once every three days for a total of three doses.

In some aspects, methods disclosed herein (e.g., administering a nucleotide vaccine encoding a tumor antigen in combination with IL-7, wherein the IL-7 is administered after the peak expansion phase of the tumor-specific immune response) comprise administering a single dose of IL-7 to a subject (e.g., suffering from a tumor). In some aspects, the subject receives multiple doses of IL-7 (i.e., repeated administration). In such aspects, the IL-7 can be administered at a dosing frequency of once a week, once in two weeks, once in three weeks, once in four weeks, once in five weeks, once in six weeks, once in seven weeks, once in eight weeks, once in nine weeks, once in 10 weeks, once in 11 weeks, or once in 12 weeks.

In some aspects, methods disclosed herein comprise administering to a subject (i) three doses of a nucleotide vaccine (e.g., encoding a tumor antigen) at a dosing frequency of once every three days, and (ii) a single dose of IL-7 after the peak expansion phase of the tumor-specific T cell immune response. In certain aspects, the single dose of IL-7 is administered at day 13 post initial administration of the nucleotide vaccine.

In some aspects, methods provided herein comprise administering multiple doses of a nucleotide vaccine (e.g., encoding a tumor antigen) in combination with an IL-7 protein, wherein the IL-7 protein is administered after at least one of the multiple doses of the nucleotide vaccine. In some aspects, the IL-7 protein is administered within about four weeks after at least one of the multiple doses of the nucleotide vaccine. In some aspects, the IL-7 protein is administered within about three weeks after at least one of the multiple doses of the nucleotide vaccine. In some aspects, the IL-7 protein is administered within about two weeks after at least one of the multiple doses of the nucleotide vaccine. In some aspects, the IL-7 protein is administered within about 13 days after at least one of the multiple doses of the nucleotide vaccine. In some aspects, the IL-7 protein is administered within about 12 days after at least one of the multiple doses of the nucleotide vaccine. In some aspects, the IL-7 protein is administered within about 11 days after at least one of the multiple doses of the nucleotide vaccine. In some aspects, the IL-7 protein is administered within about 10 days after at least one of the multiple doses of the nucleotide vaccine. In some aspects, the IL-7 protein is administered within about nine days after at least one of the multiple doses of the nucleotide vaccine. In some aspects, the IL-7 protein is administered within about eight days after at least one of the multiple doses of the nucleotide vaccine. In some aspects, the IL-7 protein is administered within about one week after at least one of the multiple doses of the nucleotide vaccine. In some aspects, the IL-7 protein is administered within about six days after at least one of the multiple doses of the nucleotide vaccine. In some aspects, the IL-7 protein is administered within about five days after at least one of the multiple doses of the nucleotide vaccine. In some aspects, the IL-7 protein is administered within about four days after at least one of the multiple doses of the nucleotide vaccine. In some aspects, the IL-7 protein is administered within about three days after at least one of the multiple doses of the nucleotide vaccine. In some aspects, the IL-7 protein is administered within about two days after at least one of the multiple doses of the nucleotide vaccine. In some aspects, the IL-7 protein is administered within about one day after at least one of the multiple doses of the nucleotide vaccine. In some aspects, the IL-7 protein is administered to the subject concurrently with at least one of the multiple doses of the nucleotide vaccine.

In some aspects, methods provided herein comprise administering to a subject (i) three doses of a nucleotide vaccine (e.g., encoding a tumor antigen) at a dosing frequency of once every three days, and (ii) an IL-7 protein, wherein the IL-7 protein is administered within about two days after the administration of at least one of the doses of the nucleotide vaccine. In some aspects, methods provided herein comprise administering to a subject (i) three doses of a nucleotide vaccine (e.g., encoding a tumor antigen) at a dosing frequency of once every three to five days, and (ii) an IL-7 protein, wherein the IL-7 protein is administered within about one day after the administration of at least one of the doses of the nucleotide vaccine. In some aspects, methods provided herein comprise administering to a subject (i) three doses of a nucleotide vaccine (e.g., encoding a tumor antigen) at a dosing frequency of once every three days, and (ii) an IL-7 protein, wherein the IL-7 protein is administered concurrently with at least one of the doses of the nucleotide vaccine.

In some aspects, the IL-7 protein is administered to the subject after administering all the multiple doses of the nucleotide vaccine. In some aspects, the IL-7 protein is administered within about four weeks after administering all the multiple doses of the nucleotide vaccine. In some aspects, the IL-7 protein is administered within about three weeks after administering all the multiple doses of the nucleotide vaccine. In some aspects, the IL-7 protein is administered within about two weeks after administering all the multiple doses of the nucleotide vaccine. In some aspects, the IL-7 protein is administered within about 13 days after administering all the multiple doses of the nucleotide vaccine. In some aspects, the IL-7 protein is administered within about 12 days after administering all the multiple doses of the nucleotide vaccine. In some aspects, the IL-7 protein is administered within about 11 days after administering all the multiple doses of the nucleotide vaccine. In some aspects, the IL-7 protein is administered within about 10 days after administering all the multiple doses of the nucleotide vaccine. In some aspects, the IL-7 protein is administered within about nine days after administering all the multiple doses of the nucleotide vaccine. In some aspects, the IL-7 protein is administered within about eight days after administering all the multiple doses of the nucleotide vaccine. In some aspects, the IL-7 protein is administered within about one week after administering all the multiple doses of the nucleotide vaccine. In some aspects, the IL-7 protein is administered within about six days after administering all the multiple doses of the nucleotide vaccine. In some aspects, the IL-7 protein is administered within about five days after administering all the multiple doses of the nucleotide vaccine. In some aspects, the IL-7 protein is administered within about four days after administering all the multiple doses of the nucleotide vaccine. In some aspects, the IL-7 protein is administered within about three days after administering all the multiple doses of the nucleotide vaccine. In some aspects, the IL-7 protein is administered within about two days after administering all the multiple doses of the nucleotide vaccine. In some aspects, the IL-7 protein is administered within about one day after administering all the multiple doses of the nucleotide vaccine.

In some aspects, an IL-7 protein disclosed herein can be administered to a subject at a weight-based dose. In certain aspects, an IL-7 protein can be administered at a weight-based dose between about 20 μg/kg and about 600 μg/kg. In certain aspects, an IL-7 protein of the present disclosure can be administered at a weight-based dose of about 20 μg/kg, about 60 μg/kg, about 120 μg/kg, about 240 μg/kg, about 360 μg/kg, about 480 μg/kg, or about 600 μg/kg.

In some aspects, an IL-7 protein disclosed herein can be administered to a subject at a dose greater than about 600 μg/kg. In certain aspects, an IL-7 protein is administered to a subject at a dose greater than about 600 μg/kg, greater than about 700 μg/kg, greater than about 800 μg/kg, greater than about 900 μg/kg, greater than about 1,000 μg/kg, greater than about 1,100 μg/kg, greater than about 1,200 μg/kg, greater than about 1,300 μg/kg, greater than about 1,400 μg/kg, greater than about 1,500 μg/kg, greater than about 1,600 μg/kg, greater than about 1,700 μg/kg, greater than about 1,800 μg/kg, greater than about 1,900 μg/kg, or greater than about 2,000 μg/kg.

In some aspects, an IL-7 protein of the present disclosure is administered at a dose of between 610 μg/kg and about 1,200 μg/kg, between 650 μg/kg and about 1,200 μg/kg, between about 700 μg/kg and about 1,200 μg/kg, between about 750 μg/kg and about 1,200 μg/kg, between about 800 μg/kg and about 1,200 μg/kg, between about 850 μg/kg and about 1,200 μg/kg, between about 900 μg/kg and about 1,200 μg/kg, between about 950 μg/kg and about 1,200 μg/kg, between about 1,000 μg/kg and about 1,200 μg/kg, between about 1,050 μg/kg and about 1,200 μg/kg, between about 1,100 μg/kg and about 1,200 μg/kg, between about 1,200 μg/kg and about 2,000 μg/kg, between about 1,300 μg/kg and about 2,000 μg/kg, between about 1,500 μg/kg and about 2,000 μg/kg, between about 1,700 μg/kg and about 2,000 μg/kg, between about 610 μg/kg and about 1,000 μg/kg, between about 650 μg/kg and about 1,000 μg/kg, between about 700 μg/kg and about 1,000 μg/kg, between about 750 μg/kg and about 1,000 μg/kg, between about 800 μg/kg and about 1,000 μg/kg, between about 850 μg/kg and about 1,000 μg/kg, between about 900 μg/kg and about 1,000 μg/kg, or between about 950 μg/kg and about 1,000 μg/kg.

In some aspects, an IL-7 protein of the present disclosure is administered at a dose of between 610 μg/kg and about 1,200 μg/kg. In certain aspects, an IL-7 protein is administered at a dose of between 650 μg/kg and about 1,200 μg/kg. In some aspects, an IL-7 protein is administered at a dose of between about 700 μg/kg and about 1,200 μg/kg. In further aspects, an IL-7 protein is administered at a dose of between about 750 μg/kg and about 1,200 μg/kg. In certain aspects, an IL-7 protein is administered at a dose of between about 800 μg/kg and about 1,200 μg/kg. In some aspects, an IL-7 protein is administered at a dose of between about 850 μg/kg and about 1,200 μg/kg. In some aspects, an IL-7 protein is administered at a dose of between about 900 μg/kg and about 1,200 μg/kg. In further aspects, an IL-7 protein is administered at a dose of between about 950 μg/kg and about 1,200 μg/kg. In some aspects, an IL-7 protein disclosed herein is administered at a dose of between about 1,000 μg/kg and about 1,200 μg/kg. In some aspects, an IL-7 protein is administered at a dose of between about 1,050 μg/kg and about 1,200 μg/kg. In some aspects, an IL-7 protein is administered at a dose of between about 1,100 μg/kg and about 1,200 μg/kg. In some aspects, an IL-7 protein is administered at a dose of between about 1,200 μg/kg and about 2,000 μg/kg. In further aspects, an IL-7 protein is administered at a dose of between about 1,300 μg/kg and about 2,000 μg/kg. In some aspects, an IL-7 protein is administered at a dose of between about 1,500 μg/kg and about 2,000 μg/kg. In some aspects, an IL-7 protein is administered at a dose of between about 1,700 μg/kg and about 2,000 μg/kg. In certain aspects, an IL-7 protein is administered at a dose of between about 610 μg/kg and about 1,000 μg/kg. In some aspects, an IL-7 protein is administered at a dose of between about 650 μg/kg and about 1,000 μg/kg. In further aspects, an IL-7 protein is administered at a dose of between about 700 μg/kg and about 1,000 μg/kg. In yet further aspects, an IL-7 protein is administered at a dose of between about 750 μg/kg and about 1,000 μg/kg. In certain aspects, an IL-7 protein is administered at a dose of between about 800 μg/kg and about 1,000 μg/kg. In some aspects, an IL-7 protein is administered at a dose of between about 850 μg/kg and about 1,000 μg/kg. In some aspects, an IL-7 protein of the present disclosure is administered at a dose of between about 900 μg/kg and about 1,000 μg/kg. In some aspects, an IL-7 protein is administered at a dose of between about 950 μg/kg and about 1,000 μg/kg.

In some aspects, an IL-7 protein is administered at a dose of between about 700 μg/kg and about 900 μg/kg, between about 750 μg/kg and about 950 μg/kg, between about 700 μg/kg and about 850 μg/kg, between about 750 μg/kg and about 850 μg/kg, between about 700 μg/kg and about 800 μg/kg, between about 800 μg/kg and about 900 μg/kg, between about 750 μg/kg and about 850 μg/kg, or between about 850 μg/kg and about 950 μg/kg. In some aspects, an IL-7 protein is administered at a dose of between about 700 μg/kg and about 900 μg/kg. In certain aspects, an IL-7 protein is administered at a dose of between about 750 μg/kg and about 950 μg/kg. In further aspects, an IL-7 protein is administered at a dose of between about 700 μg/kg and about 850 μg/kg. In some aspects, an IL-7 protein is administered at a dose of between about 750 μg/kg and about 850 μg/kg. In other aspects, an IL-7 protein is administered at a dose of between about 700 μg/kg and about 800 μg/kg. In some aspects, an IL-7 protein is administered at a dose of between about 800 μg/kg and about 900 μg/kg. In some aspects, an IL-7 protein is administered at a dose of between about 750 μg/kg and about 850 μg/kg. In certain aspects, an IL-7 protein is administered at a dose of between about 850 μg/kg and about 950 μg/kg.

In some aspects, an IL-7 protein is administered at a dose of about 650 μg/kg, about 680 μg/kg, about 700 μg/kg, about 720 μg/kg, about 740 μg/kg, about 750 μg/kg, about 760 μg/kg, about 780 μg/kg, about 800 μg/kg, about 820 μg/kg, about 840 μg/kg, about 850 μg/kg, about 860 μg/kg, about 880 μg/kg, about 900 μg/kg, about 920 μg/kg, about 940 μg/kg, about 950 μg/kg, about 960 μg/kg, about 980 μg/kg, about 1,000 μg/kg, about 1,020 μg/kg, about 1,020 μg/kg, about 1,040 μg/kg, about 1,060 μg/kg, about 1,080 μg/kg, about 1,100 μg/kg, about 1,120 μg/kg, about 1,140 μg/kg, about 1,160 μg/kg, about 1,180 μg/kg, about 1200 μg/kg, about 1,220 μg/kg, about 1,240 μg/kg, about 1,260 μg/kg, about 1,280 μg/kg, about 1,300 μg/kg, about 1,320 μg/kg, about 1,340 μg/kg, about 1,360 μg/kg, about 1,380 μg/kg, about 1,400 μg/kg, about 1,420 μg/kg, about 1,440 μg/kg, about 1,460 μg/kg, about 1,480 μg/kg, about 1,500 μg/kg, about 1,520 μg/kg, about 1,540 μg/kg, about 1,560 μg/kg, about 1,580 μg/kg, about 1,600 μg/kg, about 1,620 μg/kg, about 1,640 μg/kg, about 1,660 μg/kg, about 1,680 μg/kg, about 1,700 μg/kg, about 1,720 μg/kg, about 1,740 μg/kg, about 1,760 μg/kg, about 1,780 μg/kg, about 1,800 μg/kg, about 1,820 μg/kg, about 1,840 μg/kg, about 1,860 μg/kg, about 1,880 μg/kg, about 1,900 μg/kg, about 1,920 μg/kg, about 1,940 μg/kg, about 1,960 μg/kg, about 1,980 μg/kg, or about 2,000 μg/kg.

In some aspects, an IL-7 protein is administered at a dose of about 650 μg/kg. In other aspects, an IL-7 protein disclosed herein is administered at a dose of about 680 μg/kg. In some aspects, an IL-7 protein is administered at a dose of about 700 μg/kg. In some aspects, an IL-7 protein is administered at a dose of about 720 μg/kg. In certain aspects, an IL-7 protein is administered at a dose of about 740 μg/kg. In some aspects, an IL-7 protein is administered at a dose of about 750 μg/kg. In some aspects, an IL-7 protein is administered at a dose of about 760 μg/kg. In certain aspects, an IL-7 protein is administered at a dose of about 780 μg/kg. In some aspects, an IL-7 protein is administered at a dose of about 800 μg/kg. In further aspects, an IL-7 protein is administered at a dose of about 820 μg/kg. In certain aspects, an IL-7 protein is administered at a dose of about 840 μg/kg. In some aspects, an IL-7 protein is administered at a dose of about 850 μg/kg. In certain aspects, an IL-7 protein is administered at a dose of about 860 μg/kg. In some aspects, an IL-7 protein is administered at a dose of about 880 μg/kg. In some aspects, an IL-7 protein is administered at a dose of about 900 μg/kg. In further aspects, an IL-7 protein is administered at a dose of about 920 μg/kg. In some aspects, an IL-7 protein is administered at a dose of about 940 μg/kg. In further aspects, an IL-7 protein is administered at a dose of about 950 μg/kg. In some aspects, an IL-7 protein is administered at a dose of about 960 μg/kg. In certain aspects, an IL-7 protein is administered at a dose of about 980 μg/kg. In some aspects, an IL-7 protein is administered at a dose of about 1,000 μg/kg. In certain aspects, an IL-7 protein is administered at a dose of about 1,020 μg/kg. In further aspects, an IL-7 protein is administered at a dose of about 1,040 μg/kg. In some aspects, an IL-7 protein is administered at a dose of about 1,060 μg/kg. In other aspects, an IL-7 protein is administered at a dose of about 1,080 μg/kg. In some aspects, an IL-7 protein is administered at a dose of about 1,100 μg/kg. In certain aspects, an IL-7 protein is administered at a dose of about 1,120 μg/kg. In further aspects, an IL-7 protein is administered at a dose of about 1,140 μg/kg. In some aspects, an IL-7 protein is administered at a dose of about 1,160 μg/kg. In other aspects, an IL-7 protein is administered at a dose of about 1,180 μg/kg. In certain aspects, an IL-7 protein is administered at a dose of about 1,200 μg/kg. In certain aspects, an IL-7 protein is administered at a dose of about 1,220 μg/kg. In further aspects, an IL-7 protein is administered at a dose of about 1,240 μg/kg. In some aspects, an IL-7 protein is administered at a dose of about 1,260 μg/kg. In other aspects, an IL-7 protein is administered at a dose of about 1,280 μg/kg. In some aspects, an IL-7 protein is administered at a dose of about 1,300 μg/kg. In certain aspects, an IL-7 protein is administered at a dose of about 1,320 μg/kg. In further aspects, an IL-7 protein is administered at a dose of about 1,340 μg/kg. In some aspects, an IL-7 protein is administered at a dose of about 1,360 μg/kg. In other aspects, an IL-7 protein is administered at a dose of about 1,380 μg/kg. In further aspects, an IL-7 protein is administered at a dose of about 1,400 μg/kg. In certain aspects, an IL-7 protein is administered at a dose of about 1,420 μg/kg. In some aspects, an IL-7 protein is administered at a dose of about 1,440 μg/kg. In some aspects, an IL-7 protein is administered at a dose of about 1,460 μg/kg. In other aspects, an IL-7 protein is administered at a dose of about 1,480 μg/kg. In certain aspects, an IL-7 protein is administered at a dose of about 1,500 μg/kg. In certain aspects, an IL-7 protein is administered at a dose of about 1,520 μg/kg. In further aspects, an IL-7 protein is administered at a dose of about 1,540 μg/kg. In some aspects, an IL-7 protein is administered at a dose of about 1,560 μg/kg. In other aspects, an IL-7 protein is administered at a dose of about 1,580 μg/kg. In some aspects, an IL-7 protein is administered at a dose of about 1,600 μg/kg. In certain aspects, an IL-7 protein is administered at a dose of about 1,620 μg/kg. In further aspects, an IL-7 protein is administered at a dose of about 1,640 μg/kg. In some aspects, an IL-7 protein is administered at a dose of about 1,660 μg/kg. In other aspects, an IL-7 protein is administered at a dose of about 1,680 μg/kg. In certain aspects, an IL-7 protein is administered at a dose of about 1,700 μg/kg. In certain aspects, an IL-7 protein is administered at a dose of about 1,720 μg/kg. In further aspects, an IL-7 protein is administered at a dose of about 1,740 μg/kg. In some aspects, an IL-7 protein is administered at a dose of about 1,760 μg/kg. In other aspects, an IL-7 protein is administered at a dose of about 1,780 μg/kg. In some aspects, an IL-7 protein is administered at a dose of about 1,800 μg/kg. In certain aspects, an IL-7 protein is administered at a dose of about 1,820 μg/kg. In further aspects, an IL-7 protein is administered at a dose of about 1,840 μg/kg. In some aspects, an IL-7 protein is administered at a dose of about 1,860 μg/kg. In other aspects, an IL-7 protein is administered at a dose of about 1,880 μg/kg. In some aspects, an IL-7 protein is administered at a dose of about 1,900 μg/kg. In certain aspects, an IL-7 protein is administered at a dose of about 1,920 μg/kg. In further aspects, an IL-7 protein is administered at a dose of about 1,940 μg/kg. In some aspects, an IL-7 protein is administered at a dose of about 1,960 μg/kg. In other aspects, an IL-7 protein is administered at a dose of about 1,980 μg/kg. In further aspects, an IL-7 protein is administered at a dose of about 2,000 μg/kg.

In some aspects, an IL-7 protein is administered at a dosing frequency of about once a week, about once in two weeks, about once in three weeks, about once in four weeks, about once in five weeks, about once in six weeks, about once in seven weeks, about once in eight weeks, about once in nine weeks, about once in 10 weeks, about once in 11 weeks, or about once in 12 weeks. In certain aspects, an IL-7 protein is administered at a dosing frequency of about once every 10 days, about once every 20 days, about once every 30 days, about once every 40 days, about once every 50 days, about once every 60 days, about once every 70 days, about once every 80 days, about once every 90 days, or about once every 100 days. In some aspects, the IL-7 protein is administered once in three weeks. In some aspects, the IL-7 protein is administered once a week. In some aspects, the IL-7 protein is administered once in two weeks. In certain aspects, the IL-7 protein is administered once in three weeks. In some aspects, the IL-7 protein is administered once in four weeks. In certain aspects, the IL-7 protein is administered once in six weeks. In further aspects, the IL-7 protein is administered once in eight weeks. In some aspects, the IL-7 protein is administered once in nine weeks. In certain aspects, the IL-7 protein is administered once in 12 weeks. In some aspects, the IL-7 protein is administered once every 10 days. In certain aspects, the IL-7 protein is administered once every 20 days. In other aspects, the IL-7 protein is administered once every 30 days. In some aspects, the IL-7 protein is administered once every 40 days. In certain aspects, the IL-7 protein is administered once every 50 days. In some aspects, the IL-7 protein is administered once every 60 days. In further aspects, the IL-7 protein is administered once every 70 days. In some aspects, the IL-7 protein is administered once every 80 days. In certain aspects, the IL-7 protein is administered once every 90 days. In some aspects, the IL-7 protein is administered once every 100 days.

In some aspects, the IL-7 protein is administered twice or more times in an amount of about 720 μg/kg at an interval of about 1 week, about 2 weeks, about 3 weeks, or about 4 weeks. In some aspects, the IL-7 protein is administered twice or more times in an amount of about 840 μg/kg at an interval of about 2 weeks, about 3 weeks, about 4 weeks, or about 5 weeks. In some aspects, the IL-7 protein is administered twice or more times in an amount of about 960 μg/kg at an interval of about 2 weeks, about 3 weeks, about 4 weeks, about 5 weeks, or about 6 weeks. In some aspects, the IL-7 protein is administered twice or more times in an amount of about 1200 μg/kg at an interval of about 3 weeks, about 4 weeks, about 5 weeks, about 6 weeks, about 7 weeks, or about 8 weeks. In some aspects, the IL-7 protein is administered twice or more times in an amount of about 1440 μg/kg at an interval of about 3 weeks, about 4 weeks, about 5 weeks, about 6 weeks, about 7 weeks, about 8 weeks, about 2 months, about 8 weeks, about 10 weeks, about 12 weeks, or about 3 months.

In some aspects, the IL-7 protein is administered at a dose of 60 μg/kg with a dosing frequency of once a week. In some aspects, the IL-7 protein is administered at a dose of 120 μg/kg with a dosing frequency of once a week. In some aspects, the IL-7 protein is administered at a dose of 240 μg/kg with a dosing frequency of once a week. In some aspects, the IL-7 protein is administered at a dose of 480 μg/kg with a dosing frequency of once a week. In some aspects, the IL-7 protein is administered at a dose of 720 μg/kg with a dosing frequency of once a week. In some aspects, the IL-7 protein is administered at a dose of 960 μg/kg with a dosing frequency of once a week. In some aspects, the IL-7 protein is administered at a dose of 1,200 μg/kg with a dosing frequency of once a week. In some aspects, the IL-7 protein is administered at a dose of 1,300 μg/kg with a dosing frequency of once a week. In some aspects, the IL-7 protein is administered at a dose of 1,400 μg/kg with a dosing frequency of once a week. In other aspects, the IL-7 protein is administered at a dose of 1,420 μg/kg with a dosing frequency of once a week. In certain aspects, the IL-7 protein is administered at a dose of 1,440 μg/kg with a dosing frequency of once a week. In further aspects, the IL-7 protein is administered at a dose of 1,460 μg/kg with a dosing frequency of once a week. In certain aspects, the IL-7 protein is administered at a dose of 1,480 μg/kg with a dosing frequency of once a week. In some aspects, the IL-7 protein is administered at a dose of 1,500 μg/kg with a dosing frequency of once a week. In further aspects, the IL-7 protein is administered at a dose of 1,600 μg/kg with a dosing frequency of once a week. In some aspects, the IL-7 protein is administered at a dose of 1,700 μg/kg with a dosing frequency of once a week. In some aspects, the IL-7 protein is administered at a dose of 2,000 μg/kg with a dosing frequency of once a week.

In some aspects, the IL-7 protein is administered at a dose of 60 μg/kg with a dosing frequency of once in two weeks. In some aspects, the IL-7 protein is administered at a dose of 120 μg/kg with a dosing frequency of once in two weeks. In some aspects, the IL-7 protein is administered at a dose of 240 μg/kg with a dosing frequency of once in two weeks. In some aspects, the IL-7 protein is administered at a dose of 480 μg/kg with a dosing frequency of once in two weeks. In some aspects, the IL-7 protein is administered at a dose of 720 μg/kg with a dosing frequency of once in two weeks. In some aspects, the IL-7 protein is administered at a dose of 960 μg/kg with a dosing frequency of once in two weeks. In some aspects, the IL-7 protein is administered at a dose of 1,200 μg/kg with a dosing frequency of once in two weeks. In some aspects, the IL-7 protein is administered at a dose of 1,300 μg/kg with a dosing frequency of once in two weeks. In some aspects, the IL-7 protein is administered at a dose of 1,400 μg/kg with a dosing frequency of once in two weeks. In other aspects, the IL-7 protein is administered at a dose of 1,420 μg/kg with a dosing frequency of once in two weeks. In certain aspects, the IL-7 protein is administered at a dose of 1,440 μg/kg with a dosing frequency of once in two weeks. In further aspects, the IL-7 protein is administered at a dose of 1,460 μg/kg with a dosing frequency of once in two weeks. In certain aspects, the IL-7 protein is administered at a dose of 1,480 μg/kg with a dosing frequency of once in two weeks. In some aspects, the IL-7 protein is administered at a dose of 1,500 μg/kg with a dosing frequency of once in two weeks. In further aspects, the IL-7 protein is administered at a dose of 1,600 μg/kg with a dosing frequency of once in two weeks. In some aspects, the IL-7 protein is administered at a dose of 1,700 μg/kg with a dosing frequency of once in two weeks. In some aspects, the IL-7 protein is administered at a dose of 2,000 μg/kg with a dosing frequency of once in two weeks.

In some aspects, the IL-7 protein is administered at a dose of 60 μg/kg with a dosing frequency of once in three weeks. In some aspects, the IL-7 protein is administered at a dose of 120 μg/kg with a dosing frequency of once in three weeks. In some aspects, the IL-7 protein is administered at a dose of 240 μg/kg with a dosing frequency of once in three weeks. In some aspects, the IL-7 protein is administered at a dose of 480 μg/kg with a dosing frequency of once in three weeks. In some aspects, the IL-7 protein is administered at a dose of 720 μg/kg with a dosing frequency of once in three weeks. In some aspects, the IL-7 protein is administered at a dose of 960 μg/kg with a dosing frequency of once in three weeks. In some aspects, the IL-7 protein is administered at a dose of 1,200 μg/kg with a dosing frequency of once in three weeks. In some aspects, the IL-7 protein is administered at a dose of 1,300 μg/kg with a dosing frequency of once in three weeks. In some aspects, the IL-7 protein is administered at a dose of 1,400 μg/kg with a dosing frequency of once in three weeks. In other aspects, the IL-7 protein is administered at a dose of 1,420 μg/kg with a dosing frequency of once in three weeks. In certain aspects, the IL-7 protein is administered at a dose of 1,440 μg/kg with a dosing frequency of once in three weeks. In further aspects, the IL-7 protein is administered at a dose of 1,460 μg/kg with a dosing frequency of once in three weeks. In certain aspects, the IL-7 protein is administered at a dose of 1,480 μg/kg with a dosing frequency of once in three weeks. In some aspects, the IL-7 protein is administered at a dose of 1,500 μg/kg with a dosing frequency of once in three weeks. In further aspects, the IL-7 protein is administered at a dose of 1,600 μg/kg with a dosing frequency of once in three weeks. In some aspects, the IL-7 protein is administered at a dose of 1,700 μg/kg with a dosing frequency of once in three weeks. In some aspects, the IL-7 protein is administered at a dose of 2,000 μg/kg with a dosing frequency of once in three weeks.

In some aspects, the IL-7 protein is administered at a dose of 60 μg/kg with a dosing frequency of once in four weeks. In some aspects, the IL-7 protein is administered at a dose of 120 μg/kg with a dosing frequency of once in four weeks. In some aspects, the IL-7 protein is administered at a dose of 240 μg/kg with a dosing frequency of once in four weeks. In some aspects, the IL-7 protein is administered at a dose of 480 μg/kg with a dosing frequency of once in four weeks. In some aspects, the IL-7 protein is administered at a dose of 720 μg/kg with a dosing frequency of once in four weeks. In some aspects, the IL-7 protein is administered at a dose of 960 μg/kg with a dosing frequency of once in four weeks. In some aspects, the IL-7 protein is administered at a dose of 1,200 μg/kg with a dosing frequency of once in four weeks. In some aspects, the IL-7 protein is administered at a dose of 1,300 μg/kg with a dosing frequency of once in four weeks. In some aspects, the IL-7 protein is administered at a dose of 1,400 μg/kg with a dosing frequency of once in four weeks. In other aspects, the IL-7 protein is administered at a dose of 1,420 μg/kg with a dosing frequency of once in four weeks. In certain aspects, the IL-7 protein is administered at a dose of 1,440 μg/kg with a dosing frequency of once in four weeks. In further aspects, the IL-7 protein is administered at a dose of 1,460 μg/kg with a dosing frequency of once in four weeks. In certain aspects, the IL-7 protein is administered at a dose of 1,480 μg/kg with a dosing frequency of once in four weeks. In some aspects, the IL-7 protein is administered at a dose of 1,500 μg/kg with a dosing frequency of once in four weeks. In further aspects, the IL-7 protein is administered at a dose of 1,600 μg/kg with a dosing frequency of once in four weeks. In some aspects, the IL-7 protein is administered at a dose of 1,700 μg/kg with a dosing frequency of once in four weeks. In some aspects, the IL-7 protein is administered at a dose of 2,000 μg/kg with a dosing frequency of once in four weeks.

In some aspects, the IL-7 protein is administered at a dose of 60 μg/kg with a dosing frequency of once in five weeks. In some aspects, the IL-7 protein is administered at a dose of 120 μg/kg with a dosing frequency of once in five weeks. In some aspects, the IL-7 protein is administered at a dose of 240 μg/kg with a dosing frequency of once in five weeks. In some aspects, the IL-7 protein is administered at a dose of 480 μg/kg with a dosing frequency of once in five weeks. In some aspects, the IL-7 protein is administered at a dose of 720 μg/kg with a dosing frequency of once in five weeks. In some aspects, the IL-7 protein is administered at a dose of 960 μg/kg with a dosing frequency of once in five weeks. In some aspects, the IL-7 protein is administered at a dose of 1,200 μg/kg with a dosing frequency of once in five weeks. In some aspects, the IL-7 protein is administered at a dose of 1,300 μg/kg with a dosing frequency of once in five weeks. In some aspects, the IL-7 protein is administered at a dose of 1,400 μg/kg with a dosing frequency of once in five weeks. In other aspects, the IL-7 protein is administered at a dose of 1,420 μg/kg with a dosing frequency of once in five weeks. In certain aspects, the IL-7 protein is administered at a dose of 1,440 μg/kg with a dosing frequency of once in five weeks. In further aspects, the IL-7 protein is administered at a dose of 1,460 μg/kg with a dosing frequency of once in five weeks. In certain aspects, the IL-7 protein is administered at a dose of 1,480 μg/kg with a dosing frequency of once in five weeks. In some aspects, the IL-7 protein is administered at a dose of 1,500 μg/kg with a dosing frequency of once in five weeks. In further aspects, the IL-7 protein is administered at a dose of 1,600 μg/kg with a dosing frequency of once in five weeks. In some aspects, the IL-7 protein is administered at a dose of 1,700 μg/kg with a dosing frequency of once in five weeks. In some aspects, the IL-7 protein is administered at a dose of 2,000 μg/kg with a dosing frequency of once in five weeks.

In some aspects, the IL-7 protein is administered at a dose of 60 μg/kg with a dosing frequency of once in six weeks. In some aspects, the IL-7 protein is administered at a dose of 120 μg/kg with a dosing frequency of once in six weeks. In some aspects, the IL-7 protein is administered at a dose of 240 μg/kg with a dosing frequency of once in six weeks. In some aspects, the IL-7 protein is administered at a dose of 480 μg/kg with a dosing frequency of once in six weeks. In some aspects, the IL-7 protein is administered at a dose of 720 μg/kg with a dosing frequency of once in six weeks. In some aspects, the IL-7 protein is administered at a dose of 960 μg/kg with a dosing frequency of once in six weeks. In some aspects, the IL-7 protein is administered at a dose of 1,200 μg/kg with a dosing frequency of once in six weeks. In some aspects, the IL-7 protein is administered at a dose of 1,300 μg/kg with a dosing frequency of once in six weeks. In some aspects, the IL-7 protein is administered at a dose of 1,400 μg/kg with a dosing frequency of once in six weeks. In other aspects, the IL-7 protein is administered at a dose of 1,420 μg/kg with a dosing frequency of once in six weeks. In certain aspects, the IL-7 protein is administered at a dose of 1,440 μg/kg with a dosing frequency of once in six weeks. In further aspects, the IL-7 protein is administered at a dose of 1,460 μg/kg with a dosing frequency of once in six weeks. In certain aspects, the IL-7 protein is administered at a dose of 1,480 μg/kg with a dosing frequency of once in six weeks. In some aspects, the IL-7 protein is administered at a dose of 1,500 μg/kg with a dosing frequency of once in six weeks. In further aspects, the IL-7 protein is administered at a dose of 1,600 μg/kg with a dosing frequency of once in six weeks. In some aspects, the IL-7 protein is administered at a dose of 1,700 μg/kg with a dosing frequency of once in six weeks. In some aspects, the IL-7 protein is administered at a dose of 2,000 μg/kg with a dosing frequency of once in six weeks.

In some aspects, the IL-7 protein is administered at a dose of 60 μg/kg with a dosing frequency of once in seven weeks. In some aspects, the IL-7 protein is administered at a dose of 120 μg/kg with a dosing frequency of once in seven weeks. In some aspects, the IL-7 protein is administered at a dose of 240 μg/kg with a dosing frequency of once in seven weeks. In some aspects, the IL-7 protein is administered at a dose of 480 μg/kg with a dosing frequency of once in seven weeks. In some aspects, the IL-7 protein is administered at a dose of 720 μg/kg with a dosing frequency of once in seven weeks. In some aspects, the IL-7 protein is administered at a dose of 960 μg/kg with a dosing frequency of once in seven weeks. In some aspects, the IL-7 protein is administered at a dose of 1,200 μg/kg with a dosing frequency of once in seven weeks. In some aspects, the IL-7 protein is administered at a dose of 1,300 μg/kg with a dosing frequency of once in seven weeks. In some aspects, the IL-7 protein is administered at a dose of 1,400 μg/kg with a dosing frequency of once in seven weeks. In other aspects, the IL-7 protein is administered at a dose of 1,420 μg/kg with a dosing frequency of once in seven weeks. In certain aspects, the IL-7 protein is administered at a dose of 1,440 μg/kg with a dosing frequency of once in seven weeks. In further aspects, the IL-7 protein is administered at a dose of 1,460 μg/kg with a dosing frequency of once in seven weeks. In certain aspects, the IL-7 protein is administered at a dose of 1,480 μg/kg with a dosing frequency of once in seven weeks. In some aspects, the IL-7 protein is administered at a dose of 1,500 μg/kg with a dosing frequency of once in seven weeks. In further aspects, the IL-7 protein is administered at a dose of 1,600 μg/kg with a dosing frequency of once in seven weeks. In some aspects, the IL-7 protein is administered at a dose of 1,700 μg/kg with a dosing frequency of once in seven weeks. In some aspects, the IL-7 protein is administered at a dose of 2,000 μg/kg with a dosing frequency of once in seven weeks.

In some aspects, the IL-7 protein is administered at a dose of 60 μg/kg with a dosing frequency of once in eight weeks. In some aspects, the IL-7 protein is administered at a dose of 120 μg/kg with a dosing frequency of once in eight weeks. In some aspects, the IL-7 protein is administered at a dose of 240 μg/kg with a dosing frequency of once in eight weeks. In some aspects, the IL-7 protein is administered at a dose of 480 μg/kg with a dosing frequency of once in eight weeks. In some aspects, the IL-7 protein is administered at a dose of 720 μg/kg with a dosing frequency of once in eight weeks. In some aspects, the IL-7 protein is administered at a dose of 960 μg/kg with a dosing frequency of once in eight weeks. In some aspects, the IL-7 protein is administered at a dose of 1,200 μg/kg with a dosing frequency of once in eight weeks. In some aspects, the IL-7 protein is administered at a dose of 1,300 μg/kg with a dosing frequency of once in eight weeks. In some aspects, the IL-7 protein is administered at a dose of 1,400 μg/kg with a dosing frequency of once in eight weeks. In other aspects, the IL-7 protein is administered at a dose of 1,420 μg/kg with a dosing frequency of once in eight weeks. In certain aspects, the IL-7 protein is administered at a dose of 1,440 μg/kg with a dosing frequency of once in eight weeks. In further aspects, the IL-7 protein is administered at a dose of 1,460 μg/kg with a dosing frequency of once in eight weeks. In certain aspects, the IL-7 protein is administered at a dose of 1,480 μg/kg with a dosing frequency of once in eight weeks. In some aspects, the IL-7 protein is administered at a dose of 1,500 μg/kg with a dosing frequency of once in eight weeks. In further aspects, the IL-7 protein is administered at a dose of 1,600 μg/kg with a dosing frequency of once in eight weeks. In some aspects, the IL-7 protein is administered at a dose of 1,700 μg/kg with a dosing frequency of once in eight weeks. In some aspects, the IL-7 protein is administered at a dose of 2,000 μg/kg with a dosing frequency of once in eight weeks.

In some aspects, the IL-7 protein is administered at a dose of 60 μg/kg with a dosing frequency of once in nine weeks. In some aspects, the IL-7 protein is administered at a dose of 120 μg/kg with a dosing frequency of once in nine weeks. In some aspects, the IL-7 protein is administered at a dose of 240 μg/kg with a dosing frequency of once in nine weeks. In some aspects, the IL-7 protein is administered at a dose of 480 μg/kg with a dosing frequency of once in nine weeks. In some aspects, the IL-7 protein is administered at a dose of 720 μg/kg with a dosing frequency of once in nine weeks. In some aspects, the IL-7 protein is administered at a dose of 960 μg/kg with a dosing frequency of once in nine weeks. In some aspects, the IL-7 protein is administered at a dose of 1,200 μg/kg with a dosing frequency of once in nine weeks. In some aspects, the IL-7 protein is administered at a dose of 1,300 μg/kg with a dosing frequency of once in three weeks. In some aspects, the IL-7 protein is administered at a dose of 1,400 μg/kg with a dosing frequency of once in three weeks. In other aspects, the IL-7 protein is administered at a dose of 1,420 μg/kg with a dosing frequency of once in three weeks. In certain aspects, the IL-7 protein is administered at a dose of 1,440 μg/kg with a dosing frequency of once in nine weeks. In further aspects, the IL-7 protein is administered at a dose of 1,460 μg/kg with a dosing frequency of once in three weeks. In certain aspects, the IL-7 protein is administered at a dose of 1,480 μg/kg with a dosing frequency of once in three weeks. In some aspects, the IL-7 protein is administered at a dose of 1,500 μg/kg with a dosing frequency of once in three weeks. In further aspects, the IL-7 protein is administered at a dose of 1,600 μg/kg with a dosing frequency of once in three weeks. In some aspects, the IL-7 protein is administered at a dose of 1,700 μg/kg with a dosing frequency of once in nine weeks. In some aspects, the IL-7 protein is administered at a dose of 2,000 μg/kg with a dosing frequency of once in nine weeks.

In some aspects, the IL-7 protein is administered at a dose of 60 μg/kg with a dosing frequency of once in 10 weeks. In some aspects, the IL-7 protein is administered at a dose of 120 μg/kg with a dosing frequency of once in 10 weeks. In some aspects, the IL-7 protein is administered at a dose of 240 μg/kg with a dosing frequency of once in 10 weeks. In some aspects, the IL-7 protein is administered at a dose of 480 μg/kg with a dosing frequency of once in 10 weeks. In some aspects, the IL-7 protein is administered at a dose of 720 μg/kg with a dosing frequency of once in 10 weeks. In some aspects, the IL-7 protein is administered at a dose of 960 μg/kg with a dosing frequency of once in 10 weeks. In some aspects, the IL-7 protein is administered at a dose of 1,200 μg/kg with a dosing frequency of once in 10 weeks. In some aspects, the IL-7 protein is administered at a dose of 1,300 μg/kg with a dosing frequency of once in 10 weeks. In some aspects, the IL-7 protein is administered at a dose of 1,400 μg/kg with a dosing frequency of once in 10 weeks. In other aspects, the IL-7 protein is administered at a dose of 1,420 μg/kg with a dosing frequency of once in 10 weeks. In certain aspects, the IL-7 protein is administered at a dose of 1,440 μg/kg with a dosing frequency of once in 10 weeks. In further aspects, the IL-7 protein is administered at a dose of 1,460 μg/kg with a dosing frequency of once in 10 weeks. In certain aspects, the IL-7 protein is administered at a dose of 1,480 μg/kg with a dosing frequency of once in 10 weeks. In some aspects, the IL-7 protein is administered at a dose of 1,500 μg/kg with a dosing frequency of once in 10 weeks. In further aspects, the IL-7 protein is administered at a dose of 1,600 μg/kg with a dosing frequency of once in 10 weeks. In some aspects, the IL-7 protein is administered at a dose of 1,700 μg/kg with a dosing frequency of once in 10 weeks. In some aspects, the IL-7 protein is administered at a dose of 2,000 μg/kg with a dosing frequency of once in 10 weeks.

In some aspects, the IL-7 protein is administered at a dose of 60 μg/kg with a dosing frequency of once in 11 weeks. In some aspects, the IL-7 protein is administered at a dose of 120 μg/kg with a dosing frequency of once in 11 weeks. In some aspects, the IL-7 protein is administered at a dose of 240 μg/kg with a dosing frequency of once in 11 weeks. In some aspects, the IL-7 protein is administered at a dose of 480 μg/kg with a dosing frequency of once in 11 weeks. In some aspects, the IL-7 protein is administered at a dose of 720 μg/kg with a dosing frequency of once in 11 weeks. In some aspects, the IL-7 protein is administered at a dose of 960 μg/kg with a dosing frequency of once in 11 weeks. In some aspects, the IL-7 protein is administered at a dose of 1,200 μg/kg with a dosing frequency of once in 11 weeks. In some aspects, the IL-7 protein is administered at a dose of 1,300 μg/kg with a dosing frequency of once in 11 weeks. In some aspects, the IL-7 protein is administered at a dose of 1,400 μg/kg with a dosing frequency of once in 11 weeks. In other aspects, the IL-7 protein is administered at a dose of 1,420 μg/kg with a dosing frequency of once in 11 weeks. In certain aspects, the IL-7 protein is administered at a dose of 1,440 μg/kg with a dosing frequency of once in 11 weeks. In further aspects, the IL-7 protein is administered at a dose of 1,460 μg/kg with a dosing frequency of once in 11 weeks. In certain aspects, the IL-7 protein is administered at a dose of 1,480 μg/kg with a dosing frequency of once in 11 weeks. In some aspects, the IL-7 protein is administered at a dose of 1,500 μg/kg with a dosing frequency of once in 11 weeks. In further aspects, the IL-7 protein is administered at a dose of 1,600 μg/kg with a dosing frequency of once in 11 weeks. In some aspects, the IL-7 protein is administered at a dose of 1,700 μg/kg with a dosing frequency of once in 11 weeks. In some aspects, the IL-7 protein is administered at a dose of 2,000 μg/kg with a dosing frequency of once in 11 weeks.

In some aspects, the IL-7 protein is administered at a dose of 60 μg/kg with a dosing frequency of once in 12 weeks. In some aspects, the IL-7 protein is administered at a dose of 120 μg/kg with a dosing frequency of once in 12 weeks. In some aspects, the IL-7 protein is administered at a dose of 240 μg/kg with a dosing frequency of once in 12 weeks. In some aspects, the IL-7 protein is administered at a dose of 480 μg/kg with a dosing frequency of once in 12 weeks. In some aspects, the IL-7 protein is administered at a dose of 720 μg/kg with a dosing frequency of once in 12 weeks. In some aspects, the IL-7 protein is administered at a dose of 960 μg/kg with a dosing frequency of once in 12 weeks. In some aspects, the IL-7 protein is administered at a dose of 1,200 μg/kg with a dosing frequency of once in 12 weeks. In some aspects, the IL-7 protein is administered at a dose of 1,300 μg/kg with a dosing frequency of once in 12 weeks. In some aspects, the IL-7 protein is administered at a dose of 1,400 μg/kg with a dosing frequency of once in 12 weeks. In other aspects, the IL-7 protein is administered at a dose of 1,420 μg/kg with a dosing frequency of once in 12 weeks. In certain aspects, the IL-7 protein is administered at a dose of 1,440 μg/kg with a dosing frequency of once in 12 weeks. In further aspects, the IL-7 protein is administered at a dose of 1,460 μg/kg with a dosing frequency of once in 12 weeks. In certain aspects, the IL-7 protein is administered at a dose of 1,480 μg/kg with a dosing frequency of once in 12 weeks. In some aspects, the IL-7 protein is administered at a dose of 1,500 μg/kg with a dosing frequency of once in 12 weeks. In further aspects, the IL-7 protein is administered at a dose of 1,600 μg/kg with a dosing frequency of once in 12 weeks. In some aspects, the IL-7 protein is administered at a dose of 1,700 μg/kg with a dosing frequency of once in 12 weeks. In some aspects, the IL-7 protein is administered at a dose of 2,000 μg/kg with a dosing frequency of once in 12 weeks.

In some aspects, the IL-7 protein is administered at a dose of 60 μg/kg with a dosing frequency of once every 10 days. In some aspects, the IL-7 protein is administered at a dose of 120 μg/kg with a dosing frequency of once every 10 days. In some aspects, the IL-7 protein is administered at a dose of 240 μg/kg with a dosing frequency of once every 10 days. In some aspects, the IL-7 protein is administered at a dose of 480 μg/kg with a dosing frequency of once every 10 days. In some aspects, the IL-7 protein is administered at a dose of 720 μg/kg with a dosing frequency of once every 10 days. In some aspects, the IL-7 protein is administered at a dose of 960 μg/kg with a dosing frequency of once every 10 days. In some aspects, the IL-7 protein is administered at a dose of 1,200 μg/kg with a dosing frequency of once every 10 days. In some aspects, the IL-7 protein is administered at a dose of 1,300 μg/kg with a dosing frequency of once every 10 days. In some aspects, the IL-7 protein is administered at a dose of 1,400 μg/kg with a dosing frequency of once every 10 days. In other aspects, the IL-7 protein is administered at a dose of 1,420 μg/kg with a dosing frequency of once every 10 days. In certain aspects, the IL-7 protein is administered at a dose of 1,440 μg/kg with a dosing frequency of once every 10 days. In further aspects, the IL-7 protein is administered at a dose of 1,460 μg/kg with a dosing frequency of once every 10 days. In certain aspects, the IL-7 protein is administered at a dose of 1,480 μg/kg with a dosing frequency of once every 10 days. In some aspects, the IL-7 protein is administered at a dose of 1,500 μg/kg with a dosing frequency of once every 10 days. In further aspects, the IL-7 protein is administered at a dose of 1,600 μg/kg with a dosing frequency of once every 10 days. In some aspects, the IL-7 protein is administered at a dose of 1,700 μg/kg with a dosing frequency of once every 10 days. In some aspects, the IL-7 protein is administered at a dose of 2,000 μg/kg with a dosing frequency of once every 10 days.

In some aspects, the IL-7 protein is administered at a dose of 60 μg/kg with a dosing frequency of once every 20 days. In some aspects, the IL-7 protein is administered at a dose of 120 μg/kg with a dosing frequency of once every 20 days. In some aspects, the IL-7 protein is administered at a dose of 240 μg/kg with a dosing frequency of once every 20 days. In some aspects, the IL-7 protein is administered at a dose of 480 μg/kg with a dosing frequency of once every 20 days. In some aspects, the IL-7 protein is administered at a dose of 720 μg/kg with a dosing frequency of once every 20 days. In some aspects, the IL-7 protein is administered at a dose of 960 μg/kg with a dosing frequency of once every 20 days. In some aspects, the IL-7 protein is administered at a dose of 1,200 μg/kg with a dosing frequency of once every 20 days. In some aspects, the IL-7 protein is administered at a dose of 1,300 μg/kg with a dosing frequency of once every 20 days. In some aspects, the IL-7 protein is administered at a dose of 1,400 μg/kg with a dosing frequency of once every 20 days. In other aspects, the IL-7 protein is administered at a dose of 1,420 μg/kg with a dosing frequency of once every 20 days. In certain aspects, the IL-7 protein is administered at a dose of 1,440 μg/kg with a dosing frequency of once every 20 days. In further aspects, the IL-7 protein is administered at a dose of 1,460 μg/kg with a dosing frequency of once every 20 days. In certain aspects, the IL-7 protein is administered at a dose of 1,480 μg/kg with a dosing frequency of once every 20 days. In some aspects, the IL-7 protein is administered at a dose of 1,500 μg/kg with a dosing frequency of once every 20 days. In further aspects, the IL-7 protein is administered at a dose of 1,600 μg/kg with a dosing frequency of once every 20 days. In some aspects, the IL-7 protein is administered at a dose of 1,700 μg/kg with a dosing frequency of once every 20 days. In some aspects, the IL-7 protein is administered at a dose of 2,000 μg/kg with a dosing frequency of once every 20 days.

In some aspects, the IL-7 protein is administered at a dose of 60 μg/kg with a dosing frequency of once every 30 days. In some aspects, the IL-7 protein is administered at a dose of 120 μg/kg with a dosing frequency of once every 30 days. In some aspects, the IL-7 protein is administered at a dose of 240 μg/kg with a dosing frequency of once every 30 days. In some aspects, the IL-7 protein is administered at a dose of 480 μg/kg with a dosing frequency of once every 30 days. In some aspects, the IL-7 protein is administered at a dose of 720 μg/kg with a dosing frequency of once every 30 days. In some aspects, the IL-7 protein is administered at a dose of 960 μg/kg with a dosing frequency of once every 30 days. In some aspects, the IL-7 protein is administered at a dose of 1,200 μg/kg with a dosing frequency of once every 30 days. In some aspects, the IL-7 protein is administered at a dose of 1,300 μg/kg with a dosing frequency of once every 30 days. In some aspects, the IL-7 protein is administered at a dose of 1,400 μg/kg with a dosing frequency of once every 30 days. In other aspects, the IL-7 protein is administered at a dose of 1,420 μg/kg with a dosing frequency of once every 30 days. In certain aspects, the IL-7 protein is administered at a dose of 1,440 μg/kg with a dosing frequency of once every 30 days. In further aspects, the IL-7 protein is administered at a dose of 1,460 μg/kg with a dosing frequency of once every 30 days. In certain aspects, the IL-7 protein is administered at a dose of 1,480 μg/kg with a dosing frequency of once every 30 days. In some aspects, the IL-7 protein is administered at a dose of 1,500 μg/kg with a dosing frequency of once every 30 days. In further aspects, the IL-7 protein is administered at a dose of 1,600 μg/kg with a dosing frequency of once every 30 days. In some aspects, the IL-7 protein is administered at a dose of 1,700 μg/kg with a dosing frequency of once every 30 days. In some aspects, the IL-7 protein is administered at a dose of 2,000 μg/kg with a dosing frequency of once every 30 days.

In some aspects, the IL-7 protein is administered at a dose of 60 μg/kg with a dosing frequency of once every 40 days. In some aspects, the IL-7 protein is administered at a dose of 120 μg/kg with a dosing frequency of once every 40 days. In some aspects, the IL-7 protein is administered at a dose of 240 μg/kg with a dosing frequency of once every 40 days. In some aspects, the IL-7 protein is administered at a dose of 480 μg/kg with a dosing frequency of once every 40 days. In some aspects, the IL-7 protein is administered at a dose of 720 μg/kg with a dosing frequency of once every 40 days. In some aspects, the IL-7 protein is administered at a dose of 960 μg/kg with a dosing frequency of once every 40 days. In some aspects, the IL-7 protein is administered at a dose of 1,200 μg/kg with a dosing frequency of once every 40 days. In some aspects, the IL-7 protein is administered at a dose of 1,300 μg/kg with a dosing frequency of once every 40 days. In some aspects, the IL-7 protein is administered at a dose of 1,400 μg/kg with a dosing frequency of once every 40 days. In other aspects, the IL-7 protein is administered at a dose of 1,420 μg/kg with a dosing frequency of once every 40 days. In certain aspects, the IL-7 protein is administered at a dose of 1,440 μg/kg with a dosing frequency of once every 40 days. In further aspects, the IL-7 protein is administered at a dose of 1,460 μg/kg with a dosing frequency of once every 40 days. In certain aspects, the IL-7 protein is administered at a dose of 1,480 μg/kg with a dosing frequency of once every 40 days. In some aspects, the IL-7 protein is administered at a dose of 1,500 μg/kg with a dosing frequency of once every 40 days. In further aspects, the IL-7 protein is administered at a dose of 1,600 μg/kg with a dosing frequency of once every 40 days. In some aspects, the IL-7 protein is administered at a dose of 1,700 μg/kg with a dosing frequency of once every 40 days. In some aspects, the IL-7 protein is administered at a dose of 2,000 μg/kg with a dosing frequency of once every 40 days.

In some aspects, the IL-7 protein is administered at a dose of 60 μg/kg with a dosing frequency of once every 50 days. In some aspects, the IL-7 protein is administered at a dose of 120 μg/kg with a dosing frequency of once every 50 days. In some aspects, the IL-7 protein is administered at a dose of 240 μg/kg with a dosing frequency of once every 50 days. In some aspects, the IL-7 protein is administered at a dose of 480 μg/kg with a dosing frequency of once every 50 days. In some aspects, the IL-7 protein is administered at a dose of 720 μg/kg with a dosing frequency of once every 50 days. In some aspects, the IL-7 protein is administered at a dose of 960 μg/kg with a dosing frequency of once every 50 days. In some aspects, the IL-7 protein is administered at a dose of 1,200 μg/kg with a dosing frequency of once every 50 days. In some aspects, the IL-7 protein is administered at a dose of 1,300 μg/kg with a dosing frequency of once every 50 days. In some aspects, the IL-7 protein is administered at a dose of 1,400 μg/kg with a dosing frequency of once every 50 days. In other aspects, the IL-7 protein is administered at a dose of 1,420 μg/kg with a dosing frequency of once every 50 days. In certain aspects, the IL-7 protein is administered at a dose of 1,440 μg/kg with a dosing frequency of once every 50 days. In further aspects, the IL-7 protein is administered at a dose of 1,460 μg/kg with a dosing frequency of once every 50 days. In certain aspects, the IL-7 protein is administered at a dose of 1,480 μg/kg with a dosing frequency of once every 50 days. In some aspects, the IL-7 protein is administered at a dose of 1,500 μg/kg with a dosing frequency of once every 50 days. In further aspects, the IL-7 protein is administered at a dose of 1,600 μg/kg with a dosing frequency of once every 50 days. In some aspects, the IL-7 protein is administered at a dose of 1,700 μg/kg with a dosing frequency of once every 50 days. In some aspects, the IL-7 protein is administered at a dose of 2,000 μg/kg with a dosing frequency of once every 50 days.

In some aspects, the IL-7 protein is administered at a dose of 60 μg/kg with a dosing frequency of once every 60 days. In some aspects, the IL-7 protein is administered at a dose of 120 μg/kg with a dosing frequency of once every 60 days. In some aspects, the IL-7 protein is administered at a dose of 240 μg/kg with a dosing frequency of once every 60 days. In some aspects, the IL-7 protein is administered at a dose of 480 μg/kg with a dosing frequency of once every 60 days. In some aspects, the IL-7 protein is administered at a dose of 720 μg/kg with a dosing frequency of once every 60 days. In some aspects, the IL-7 protein is administered at a dose of 960 μg/kg with a dosing frequency of once every 60 days. In some aspects, the IL-7 protein is administered at a dose of 1,200 μg/kg with a dosing frequency of once every 60 days. In some aspects, the IL-7 protein is administered at a dose of 1,300 μg/kg with a dosing frequency of once every 60 days. In some aspects, the IL-7 protein is administered at a dose of 1,400 μg/kg with a dosing frequency of once every 60 days. In other aspects, the IL-7 protein is administered at a dose of 1,420 μg/kg with a dosing frequency of once every 60 days. In certain aspects, the IL-7 protein is administered at a dose of 1,440 μg/kg with a dosing frequency of once every 60 days. In further aspects, the IL-7 protein is administered at a dose of 1,460 μg/kg with a dosing frequency of once every 60 days. In certain aspects, the IL-7 protein is administered at a dose of 1,480 μg/kg with a dosing frequency of once every 60 days. In some aspects, the IL-7 protein is administered at a dose of 1,500 μg/kg with a dosing frequency of once every 60 days. In further aspects, the IL-7 protein is administered at a dose of 1,600 μg/kg with a dosing frequency of once every 60 days. In some aspects, the IL-7 protein is administered at a dose of 1,700 μg/kg with a dosing frequency of once every 60 days. In some aspects, the IL-7 protein is administered at a dose of 2,000 μg/kg with a dosing frequency of once every 60 days.

In some aspects, the IL-7 protein is administered at a dose of 60 μg/kg with a dosing frequency of once every 70 days. In some aspects, the IL-7 protein is administered at a dose of 120 μg/kg with a dosing frequency of once every 70 days. In some aspects, the IL-7 protein is administered at a dose of 240 μg/kg with a dosing frequency of once every 70 days. In some aspects, the IL-7 protein is administered at a dose of 480 μg/kg with a dosing frequency of once every 70 days. In some aspects, the IL-7 protein is administered at a dose of 720 μg/kg with a dosing frequency of once every 70 days. In some aspects, the IL-7 protein is administered at a dose of 960 μg/kg with a dosing frequency of once every 70 days. In some aspects, the IL-7 protein is administered at a dose of 1,200 μg/kg with a dosing frequency of once every 70 days. In some aspects, the IL-7 protein is administered at a dose of 1,300 μg/kg with a dosing frequency of once every 70 days. In some aspects, the IL-7 protein is administered at a dose of 1,400 μg/kg with a dosing frequency of once every 70 days. In other aspects, the IL-7 protein is administered at a dose of 1,420 μg/kg with a dosing frequency of once every 70 days. In certain aspects, the IL-7 protein is administered at a dose of 1,440 μg/kg with a dosing frequency of once every 70 days. In further aspects, the IL-7 protein is administered at a dose of 1,460 μg/kg with a dosing frequency of once every 70 days. In certain aspects, the IL-7 protein is administered at a dose of 1,480 μg/kg with a dosing frequency of once every 70 days. In some aspects, the IL-7 protein is administered at a dose of 1,500 μg/kg with a dosing frequency of once every 70 days. In further aspects, the IL-7 protein is administered at a dose of 1,600 μg/kg with a dosing frequency of once every 70 days. In some aspects, the IL-7 protein is administered at a dose of 1,700 μg/kg with a dosing frequency of once every 70 days. In some aspects, the IL-7 protein is administered at a dose of 2,000 μg/kg with a dosing frequency of once every 70 days.

In some aspects, the IL-7 protein is administered at a dose of 60 μg/kg with a dosing frequency of once every 80 days. In some aspects, the IL-7 protein is administered at a dose of 120 μg/kg with a dosing frequency of once every 80 days. In some aspects, the IL-7 protein is administered at a dose of 240 μg/kg with a dosing frequency of once every 80 days. In some aspects, the IL-7 protein is administered at a dose of 480 μg/kg with a dosing frequency of once every 80 days. In some aspects, the IL-7 protein is administered at a dose of 720 μg/kg with a dosing frequency of once every 80 days. In some aspects, the IL-7 protein is administered at a dose of 960 μg/kg with a dosing frequency of once every 80 days. In some aspects, the IL-7 protein is administered at a dose of 1,200 μg/kg with a dosing frequency of once every 80 days. In some aspects, the IL-7 protein is administered at a dose of 1,300 μg/kg with a dosing frequency of once every 80 days. In some aspects, the IL-7 protein is administered at a dose of 1,400 μg/kg with a dosing frequency of once every 80 days. In other aspects, the IL-7 protein is administered at a dose of 1,420 μg/kg with a dosing frequency of once every 80 days. In certain aspects, the IL-7 protein is administered at a dose of 1,440 μg/kg with a dosing frequency of once every 80 days. In further aspects, the IL-7 protein is administered at a dose of 1,460 μg/kg with a dosing frequency of once every 80 days. In certain aspects, the IL-7 protein is administered at a dose of 1,480 μg/kg with a dosing frequency of once every 80 days. In some aspects, the IL-7 protein is administered at a dose of 1,500 μg/kg with a dosing frequency of once every 80 days. In further aspects, the IL-7 protein is administered at a dose of 1,600 μg/kg with a dosing frequency of once every 80 days. In some aspects, the IL-7 protein is administered at a dose of 1,700 μg/kg with a dosing frequency of once every 80 days. In some aspects, the IL-7 protein is administered at a dose of 2,000 μg/kg with a dosing frequency of once every 80 days.

In some aspects, the IL-7 protein is administered at a dose of 60 μg/kg with a dosing frequency of once every 90 days. In some aspects, the IL-7 protein is administered at a dose of 120 μg/kg with a dosing frequency of once every 90 days. In some aspects, the IL-7 protein is administered at a dose of 240 μg/kg with a dosing frequency of once every 90 days. In some aspects, the IL-7 protein is administered at a dose of 480 μg/kg with a dosing frequency of once every 90 days. In some aspects, the IL-7 protein is administered at a dose of 720 μg/kg with a dosing frequency of once every 90 days. In some aspects, the IL-7 protein is administered at a dose of 960 μg/kg with a dosing frequency of once every 90 days. In some aspects, the IL-7 protein is administered at a dose of 1,200 μg/kg with a dosing frequency of once every 90 days. In some aspects, the IL-7 protein is administered at a dose of 1,300 μg/kg with a dosing frequency of once every 90 days. In some aspects, the IL-7 protein is administered at a dose of 1,400 μg/kg with a dosing frequency of once every 90 days. In other aspects, the IL-7 protein is administered at a dose of 1,420 μg/kg with a dosing frequency of once every 90 days. In certain aspects, the IL-7 protein is administered at a dose of 1,440 μg/kg with a dosing frequency of once every 90 days. In further aspects, the IL-7 protein is administered at a dose of 1,460 μg/kg with a dosing frequency of once every 90 days. In certain aspects, the IL-7 protein is administered at a dose of 1,480 μg/kg with a dosing frequency of once every 90 days. In some aspects, the IL-7 protein is administered at a dose of 1,500 μg/kg with a dosing frequency of once every 90 days. In further aspects, the IL-7 protein is administered at a dose of 1,600 μg/kg with a dosing frequency of once every 90 days. In some aspects, the IL-7 protein is administered at a dose of 1,700 μg/kg with a dosing frequency of once every 90 days. In some aspects, the IL-7 protein is administered at a dose of 2,000 μg/kg with a dosing frequency of once every 90 days.

In some aspects, the IL-7 protein is administered at a dose of 60 μg/kg with a dosing frequency of once every 100 days. In some aspects, the IL-7 protein is administered at a dose of 120 μg/kg with a dosing frequency of once every 100 days. In some aspects, the IL-7 protein is administered at a dose of 240 μg/kg with a dosing frequency of once every 100 days. In some aspects, the IL-7 protein is administered at a dose of 480 μg/kg with a dosing frequency of once every 100 days. In some aspects, the IL-7 protein is administered at a dose of 720 μg/kg with a dosing frequency of once every 100 days. In some aspects, the IL-7 protein is administered at a dose of 960 μg/kg with a dosing frequency of once every 100 days. In some aspects, the IL-7 protein is administered at a dose of 1,200 μg/kg with a dosing frequency of once every 100 days. In some aspects, the IL-7 protein is administered at a dose of 1,300 μg/kg with a dosing frequency of once every 100 days. In some aspects, the IL-7 protein is administered at a dose of 1,400 μg/kg with a dosing frequency of once every 100 days. In other aspects, the IL-7 protein is administered at a dose of 1,420 μg/kg with a dosing frequency of once every 100 days. In certain aspects, the IL-7 protein is administered at a dose of 1,440 μg/kg with a dosing frequency of once every 100 days. In further aspects, the IL-7 protein is administered at a dose of 1,460 μg/kg with a dosing frequency of once every 100 days. In certain aspects, the IL-7 protein is administered at a dose of 1,480 μg/kg with a dosing frequency of once every 100 days. In some aspects, the IL-7 protein is administered at a dose of 1,500 μg/kg with a dosing frequency of once every 100 days. In further aspects, the IL-7 protein is administered at a dose of 1,600 μg/kg with a dosing frequency of once every 100 days. In some aspects, the IL-7 protein is administered at a dose of 1,700 μg/kg with a dosing frequency of once every 100 days. In some aspects, the IL-7 protein is administered at a dose of 2,000 μg/kg with a dosing frequency of once every 100 days.

In some aspects, nucleotide vaccines that are useful for the present disclosure comprises a DNA vaccine, mRNA vaccine, or both. In certain aspects, the nucleotide vaccine is a DNA vaccine. In certain aspects, the nucleotide vaccine is a mRNA vaccine.

In some aspects, IL-7 is administered to a subject (e.g., suffering from a tumor) as a protein (IL-7 protein), nucleic acid encoding the IL-7 protein, or both.

A nucleotide vaccine (e.g., encoding a tumor antigen) and IL-7 described herein can be administered to a subject having a solid tumor by any relevant route of administration. In some aspects, the nucleotide vaccine and/or IL-7 is administered to the subject parenthetically, intramuscularly, cutaneously, subcutaneously, ophthalmic, intravenously, intraperitoneally, intradermally, intraorbitally, intracerebrally, intracranially, intraspinally, intraventricular, intrathecally, intracistemally, intracapsularly, or intratumorally.

In some aspects, methods disclosed herein (e.g., administering a nucleotide vaccine encoding a tumor antigen in combination with IL-7, wherein the IL-7 is administered after the peak expansion phase of a tumor-specific T cell immune response) can be used in combination with one or more additional therapeutic agent (e.g., anti-cancer and/or immunomodulating agents). Such agents can include, for example, chemotherapy drugs, small molecule drugs, or antibodies that stimulate the immune response to a given cancer. In some aspects, the methods described herein are used in combination with a standard of care treatment (e.g., surgery, radiation, and chemotherapy). Methods described herein can also be used as a maintenance therapy, e.g., a therapy that is intended to prevent the occurrence or recurrence of a tumor.

In some aspects, the one or more additional therapeutic agent comprises an immuno-oncology agent, such that multiple elements of the immune pathway can be targeted. Non-limiting of such combinations include: a therapy that enhances tumor antigen presentation (e.g., dendritic cell vaccine, GM-CSF secreting cellular vaccines, CpG oligonucleotides, imiquimod); a therapy that inhibits negative immune regulation e.g., by inhibiting CTLA-4 and/or PD-1/PD-L1/PD-L2 pathway and/or depleting or blocking Tregs or other immune suppressing cells (e.g., myeloid-derived suppressor cells); a therapy that stimulates positive immune regulation, e.g., with agonists that stimulate the CD-137, OX-40, and/or CD40 or GITR pathway and/or stimulate T cell effector function; a therapy that increases systemically the frequency of anti-tumor T cells; a therapy that depletes or inhibits Tregs, such as Tregs in the tumor, e.g., using an antagonist of CD25 (e.g., daclizumab) or by ex vivo anti-CD25 bead depletion; a therapy that impacts the function of suppressor myeloid cells in the tumor; a therapy that enhances immunogenicity of tumor cells (e.g., anthracyclines); adoptive T cell or NK cell transfer including genetically modified cells, e.g., cells modified by chimeric antigen receptors (CAR-T therapy); a therapy that inhibits a metabolic enzyme such as indoleamine dioxigenase (IDO), dioxigenase, arginase, or nitric oxide synthetase; a therapy that reverses/prevents T cell anergy or exhaustion; a therapy that triggers an innate immune activation and/or inflammation at a tumor site; administration of immune stimulatory cytokines; or blocking of immuno repressive cytokines.

In some aspects, an immuno-oncology agent that can be used with the present disclosure comprises an immune checkpoint inhibitor (i.e., blocks signaling through the particular immune checkpoint pathway). Non-limiting examples of immune checkpoint inhibitors that can be used in the present methods comprise a CTLA-4 antagonist (e.g., anti-CTLA-4 antibody), PD-1 antagonist (e.g., anti-PD-1 antibody, anti-PD-L1 antibody), TIM-3 antagonist (e.g., anti-TIM-3 antibody), or combinations thereof.

In some aspects, an immuno-oncology agent comprises an immune checkpoint activator (i.e., promotes signaling through the particular immune checkpoint pathway). In certain aspects, immune checkpoint activator comprises OX40 agonist (e.g., anti-OX40 antibody), LAG-3 agonist (e.g. anti-LAG-3 antibody), 4-1BB (CD137) agonist (e.g., anti-CD137 antibody), GITR agonist (e.g., anti-GITR antibody), or any combination thereof.

### III. IL-7 Proteins Useful for the Disclosure

Disclosed herein are IL-7 proteins that can be used in combination with a nucleotide vaccine (e.g., encoding a tumor antigen), e.g., to treat a tumor (or cancer). In some aspects, IL-7 protein useful for the present uses can be wild-type IL-7 or modified IL-7 (i.e., not wild-type IL-7 protein) (e.g., IL-7 variant, IL-7 functional fragment, IL-7 derivative, or any combination thereof, e.g., fusion protein, chimeric protein, etc.) as long as the IL-7 protein contains one or more biological activities of IL-7, e.g., capable of binding to IL-7R, e.g., inducing early T-cell development, promoting T-cell homeostasis. See ElKassar and Gress. J Immunotoxicol. 2010 March; 7(1): 1-7. In some aspects, an IL-7 protein of the present disclosure is not a wild-type IL-7 protein (i.e., comprises one or more modifications). Non-limiting examples of such modifications can include an oligopeptide and/or a half-life extending moiety. See WO 2016/200219, which is herein incorporated by reference in its entirety.

IL-7 binds to its receptor which is composed of the two chains IL-7Rα (CD127), shared with the thymic stromal lymphopoietin (TSLP) (Ziegler and Liu, 2006), and the common 7 chain (CD132) for IL-2, IL-15, IL-9 and IL-21. Whereas γc is expressed by most hematopoietic cells, IL-7Rα is nearly exclusively expressed on lymphoid cells. After binding to its receptor, IL-7 signals through two different pathways: Jak-Stat (Janus kinase-Signal transducer and activator of transcription) and PI3K/Akt responsible for differentiation and survival, respectively. The absence of IL-7 signaling is responsible for a reduced thymic cellularity as observed in mice that have received an anti-IL-7 neutralizing monoclonal antibody (MAb); Grabstein et al., 1993), in IL-7−/− (von Freeden-Jeffry et al., 1995), IL-7Rα−/− (Peschon et al., 1994; Maki et al., 1996), γc−/− (Malissen et al., 1997), and Jak3−/− mice (Park et al., 1995). In the absence of IL-7 signaling, mice lack T-, B-, and NK-T cells. IL-7α−/− mice (Peschon et al., 1994) have a similar but more severe phenotype than IL-7−/− mice (von Freeden-Jeffry et al., 1995), possibly because TSLP signaling is also abrogated in IL-7α−/− mice. IL-7 is required for the development of γδ cells (Maki et al., 1996) and NK-T cells (Boesteanu et al., 1997).

In some aspects, an IL-7 protein useful for the present disclosure comprises an amino acid sequence as set forth in any one of SEQ ID NOs: 1 to 6. In some aspects, the IL-7 protein comprises an amino acid sequence having a sequence identity of about 70%, about 75%, about 80%, about 85%, about 90%, about 91%, about 92%, about 93%, about 94%, about 95%, about 96%, about 97%, about 98%, or about 99% or higher, to a sequence of SEQ ID NOS: 1 to 6.

In some aspects, the IL-7 protein includes a modified IL-7 or a fragment thereof, wherein the modified IL-7 or the fragment retains one or more biological activities of wild-type IL-7. In some aspects, the IL-7 protein can be derived from humans, rats, mice, monkeys, cows, or sheep.

In some aspects, the human IL-7 can have an amino acid sequence represented by SEQ ID NO: 1 (Genbank Accession No. P13232); the rat IL-7 can have an amino acid sequence represented by SEQ ID NO: 2 (Genbank Accession No. P56478); the mouse IL-7 can have an amino acid sequence represented by SEQ ID NO: 3 (Genbank Accession No. P10168); the monkey IL-7 can have an amino acid sequence represented by SEQ ID NO: 4 (Genbank Accession No. NP 001279008); the cow IL-7 can have an amino acid sequence represented by SEQ ID NO: 5 (Genbank Accession No. P26895), and the sheep IL-7 can have an amino acid sequence represented by SEQ ID NO: 6 (Genbank Accession No. Q28540).

In some aspects, an IL-7 protein useful for the present disclosure comprises an IL-7 fusion protein. In certain aspects, an IL-7 fusion protein comprises (i) an oligopeptide and (i) an IL-7 or a variant thereof. In some aspects, the oligopeptide is linked to the N-terminal region of the IL-7 or a variant thereof.

In some aspects, an oligopeptide disclosed herein consists of 1 to 10 amino acids. In certain aspects, an oligopeptide consists of at least 1, at least 2, at least 3, at least 4, at least 5, at least 6, at least 7, at least 8, at least 9, or 10 amino acids. In some aspects, one or more amino acids of an oligopeptide are selected from the group consisting of methionine, glycine, and combinations thereof. In certain aspects, an oligopeptide is selected from the group consisting of methionine (M), glycine (G), methionine-methionine (MM), glycine-glycine (GG), methionine-glycine (MG), glycine-methionine (GM), methionine-methionine-methionine (MMM), methionine-methionine-glycine (MMG), methionine-glycine-methionine (MGM), glycine-methionine-methionine (GMM), methionine-glycine-glycine (MGG), glycine-methionine-glycine (GMG), glycine-glycine-methionine (GGM), glycine-glycine-glycine (GGG), methionine-glycine-glycine-methionine (MGGM) (SEQ ID NO: 41), methionine-methionine-glycine-glycine (MMGG) (SEQ ID NO: 42), glycine-glycine-methionine-methionine (GGMM) (SEQ ID NO: 43), methionine-glycine-methionine-glycine (MGMG) (SEQ ID NO: 44), glycine-methionine-methionine-glycine (GMMG) (SEQ ID NO: 45), glycine-glycine-glycine-methionine (GGGM) (SEQ ID NO: 46), methionine-glycine-glycine-glycine (MGGG) (SEQ ID NO: 47), glycine-methionine-glycine-glycine (GMGG) (SEQ ID NO: 48), glycine-glycine-methionine-glycine (GGMG) (SEQ ID NO: 49), glycine-glycine-methionine-methionine-methionine (GGMMM) (SEQ ID NO: 50), glycine-glycine-glycine-methionine-methionine (GGGMM) (SEQ ID NO: 51), glycine-glycine-glycine-glycine-methionine (GGGGM) (SEQ ID NO: 52), methionine-glycine-methionine-methionine-methionine (MGMMM) (SEQ ID NO: 53), methionine-glycine-glycine-methionine-methionine (MGGMM) (SEQ ID NO: 54), methionine-glycine-glycine-glycine-methionine (MGGGM) (SEQ ID NO: 55), methionine-methionine-glycine-methionine-methionine (MMGMM) (SEQ ID NO: 56), methionine-methionine-glycine-glycine-methionine (MMGGM) (SEQ ID NO: 57), methionine-methionine-glycine-glycine-glycine (MMGGG) (SEQ ID NO: 58), methionine-methionine-methionine-glycine-methionine (MMMGM) (SEQ ID NO: 59), methionine-glycine-methionine-glycine-methionine (MGMGM) (SEQ ID NO: 60), glycine-methionine-glycine-methionine-glycine (GMGMG) (SEQ ID NO: 61), glycine-methionine-methionine-methionine-glycine (GMMMG) (SEQ ID NO: 62), glycine-glycine-methionine-glycine-methionine (GGMGM) (SEQ ID NO: 63), glycine-glycine-methionine-methionine-glycine (GGMMG) (SEQ ID NO: 64), glycine-methionine-methionine-glycine-methionine (GMMGM) (SEQ ID NO: 65), methionine-glycine-methionine-methionine-glycine (MGMMG) (SEQ ID NO: 66), glycine-methionine-glycine-glycine-methionine (GMGGM) (SEQ ID NO: 67), methionine-methionine-glycine-methionine-glycine (MMGMG) (SEQ ID NO: 68), glycine-methionine-methionine-glycine-glycine (GMMGG) (SEQ ID NO: 69), glycine-methionine-glycine-glycine-glycine (GMGGG) (SEQ ID NO: 70), glycine-glycine-methionine-glycine-glycine (GGMGG) (SEQ ID NO: 71), glycine-glycine-glycine-glycine-glycine (GGGGG) (SEQ ID NO: 72), or combinations thereof. In some aspects, an oligopeptide is methionine-glycine-methionine (MGM).

In some aspects, an IL-7 fusion protein comprises (i) an IL-7 or a variant thereof, and (ii) a half-life extending moiety. In some aspects, a half-life extending moiety extends the half-life of the IL-7 or variant thereof. In some aspects, a half-life extending moiety is linked to the C-terminal region of an IL-7 or a variant thereof.

In some aspects, an IL-7 fusion protein comprises (i) IL-7 (a first domain), (ii) a second domain that includes an amino acid sequence having 1 to 10 amino acid residues consisting of methionine, glycine, or a combination thereof, e.g., MGM, and (iii) a third domain comprising a half-life extending moiety. In some aspects, the half-life extending moiety can be linked to the N-terminal or the C-terminal of the first domain or the second domain. Additionally, the IL-7 including the first domain and the second domain can be linked to both terminals of the third domain.

Non-limiting examples of half-life extending moieties include: Fc, albumin, an albumin-binding polypeptide, Pro/Ala/Ser (PAS), a C-terminal peptide (CTP) of the R subunit of human chorionic gonadotropin, polyethylene glycol (PEG), long unstructured hydrophilic sequences of amino acids (XTEN), hydroxyethyl starch (HES), an albumin-binding small molecule, and combinations thereof.

In some aspects, a half-life extending moiety is Fc. In certain aspects, Fc is from a modified immunoglobulin in which the antibody-dependent cellular cytotoxicity (ADCC) or complement-dependent cytotoxicity (CDC) weakened due to the modification in the binding affinity with the Fc receptor and/or a complement. In some aspects, the modified immunoglobulin can be selected from the group consisting of IgG1, IgG2, IgG3, IgG4, IgA1, IgA2, IgD, IgE, and a combination thereof. In some aspects, an Fc is a hybrid Fc (“hFc” or “hyFc”), comprising a hinge region, a CH2 domain, and a CH3 domain. In certain aspects, a hinge region of a hybrid Fc disclosed herein comprises a human IgD hinge region. In certain aspects, a CH2 domain of a hybrid Fc comprises a part of human IgD CH2 domain and a part of human IgG4 CH2 domain. In certain aspects, a CH3 domain of a hybrid Fc comprises a part of human IgG4 CH3 domain. Accordingly, in some aspects, a hybrid Fc disclosed herein comprises a hinge region, a CH2 domain, and a CH3 domain, wherein the hinge region comprises a human IgD hinge region, wherein the CH2 domain comprises a part of human IgD CH2 domain and a part of human IgG4 CH2 domain, and wherein the CH3 domain comprises a part of human IgG4 CH3 domain.

In some aspects, an Fc disclosed herein can be an Fc variant. As used herein, the term “Fc variant” refers to an Fc which was prepared by substituting a part of the amino acids among the Fc region or by combining the Fc regions of different kinds. The Fc region variant can prevent from being cut off at the hinge region. Specifically, in some aspects, a Fc variant comprises modifications at the 144^{th} amino acid and/or 145^{th} amino acid of SEQ ID NO: 9. In certain aspects, the 144^{th} amino acid (K) and/or the 145^{th} amino acid (K) is substituted with G or S.

In some aspects, an Fc or an Fc variant disclosed herein can be represented by the following formula: N′—(Z1)p-Y—Z2-Z3-Z4-C, wherein:
N′ comprises the N-terminus;
Z1 comprises an amino acid sequence having 5 to 9 consecutive amino acid residues from the amino acid residue at position 98 toward the N-terminal, among the amino acid residues at positions from 90 to 98 of SEQ ID NO: 7;
Y comprises an amino acid sequence having 5 to 64 consecutive amino acid residues from the amino acid residue at position 162 toward the N-terminal, among the amino acid residues at positions from 99 to 162 of SEQ ID NO: 7;
Z2 comprises an amino acid sequence having 4 to 37 consecutive amino acid residues from the amino acid residue at position 163 toward the C-terminal, among the amino acid residues at positions from 163 to 199 of SEQ ID NO: 7;
Z3 comprises an amino acid sequence having 71 to 106 consecutive amino acid residues from the amino acid residue at position 220 toward the N-terminal, among the amino acid residues at positions from 115 to 220 of SEQ ID NO: 8; and
Z4 comprises an amino acid sequence having 80 to 107 consecutive amino acid residues from the amino acid residue at position 221 toward the C-terminal, among the amino acid residues at positions from 221 to 327 of SEQ ID NO: 8.

In some aspects, a Fc region disclosed herein can include the amino acid sequence of SEQ ID NO: 9 (hyFc), SEQ ID NO: 10 (hyFcM1), SEQ ID NO: 11 (hyFcM2), SEQ ID NO: 12 (hyFcM3), or SEQ ID NO: 13 (hyFcM4). In some aspects, the Fc region can include the amino acid sequence of SEQ ID NO: 14 (a non-lytic mouse Fc).

Other non-limiting examples of Fc regions that can be used with the present disclosure are described in U.S. Pat. No. 7,867,491, which is herein incorporated by reference in its entirety.

In some aspects, an IL-7 fusion protein disclosed herein comprises both an oligopeptide and a half-life extending moiety.

In some aspects, an IL-7 protein can be fused to albumin, a variant, or a fragment thereof. Examples of the IL-7-albumin fusion protein can be found at International Application Publication No. WO 2011/124718 A1. In some aspects, an IL-7 protein is fused to a pre-pro-B cell Growth Stimulating Factor (PPBSF), optionally by a flexible linker. See US 2002/0058791A1. In some aspects, an IL-7 protein useful for the disclosure is an IL-7 conformer that has a particular three dimensional structure. See US 2005/0249701 A1. In some aspects, an IL-7 protein can be fused to an Ig chain, wherein amino acid residues 70 and 91 in the IL-7 protein are glycosylated the amino acid residue 116 in the IL-7 protein is non-glycosylated. See U.S. Pat. No. 7,323,549 B2. In some aspects, an IL-7 protein that does not contain potential T-cell epitopes (thereby to reduce anti-IL-7 T-cell responses) can also be used for the present disclosure. See US 2006/0141581 A1. In some aspects, an IL-7 protein that has one or more amino acid residue mutations in carboxy-terminal helix D region can be used for the present disclosure. The IL-7 mutant can act as IL-7R partial agonist despite lower binding affinity for the receptor. See US 2005/0054054A1. Any IL-7 proteins described in the above listed patents or publications are incorporated herein by reference in their entireties.

In addition, non-limiting examples of additional IL-7 proteins useful for the present disclosure are described in U.S. Pat. Nos. 7,708,985, 8,034,327, 8,153,114, 7,589,179, 7,323,549, 7,960,514, 8,338,575, 7,118,754, 7,488,482, 7,670,607, 6,730,512, WO0017362, GB2434578A, WO 2010/020766 A2, WO91/01143, Beq et al., Blood, vol. 114 (4), 816, 23 Jul. 2009, Kang et al., J. Virol. Doi:10.1128/JVI.02768-15, Martin et al., Blood, vol. 121 (22), 4484, May 30, 2013, McBride et al., Acta Oncologica, 34:3, 447-451, Jul. 8, 2009, and Xu et al., Cancer Science, 109: 279-288, 2018, which are incorporated herein by reference in their entireties.

In some aspects, an oligopeptide disclosed herein is directly linked to the N-terminal region of IL-7 or a variant thereof. In some aspects, an oligopeptide is linked to the N-terminal region via a linker. In some aspects, a half-life extending moiety disclosed herein is directly linked to the C-terminal region of IL-7 or a variant thereof. In certain aspects, a half-life extending moiety is linked to the C-terminal region via a linker. In some aspects, a linker is a peptide linker. In certain aspects, a peptide linker comprises a peptide of 10 to 20 amino acid residues consisting of Gly and Ser residues. In some aspects, a linker is an albumin linker. In some aspects, a linker is a chemical bond. In certain aspects, a chemical bond comprises a disulfide bond, a diamine bond, a sulfide-amine bond, a carboxy-amine bond, an ester bond, a covalent bond, or combinations thereof. When the linker is a peptide linker, in some aspects, the connection can occur in any linking region. They can be coupled using a crosslinking agent known in the art. In some aspects, examples of the crosslinking agent can include N-hydroxy succinimide esters such as 1,1-bis(diazoacetyl)-2-phenylethane, glutaraldehyde, and 4-azidosalicylic acid; imido esters including disuccinimidyl esters such as 3,3′-dithiobis (succinimidyl propionate), and bifunctional maleimides such as bis-Nmaleimido-1,8-octane, but is not limited thereto.

In some aspects, an IL-7 (or variant thereof) portion of IL-7 fusion protein disclosed herein comprises an amino sequence that is at least 70%, at least 75%, at least 80%, at least 85%, at least 90%, at least 91%, at least 92%, at least 93%, at least 94%, at least 95%, at least 96%, at least 97%, at least 98%, at least 98%, or at least 99% identical to an amino acid sequence set forth in SEQ ID NOs: 15-20. In certain aspects, an IL-7 (or variant thereof) portion of IL-7 fusion protein disclosed herein comprises the amino acid sequence set forth in SEQ ID NOs: 15-20.

In some aspects, an IL-7 fusion protein comprises: a first domain including a polypeptide having the activity of IL-7 or a similar activity thereof; a second domain comprising an amino acid sequence having 1 to 10 amino acid residues consisting of methionine, glycine, or a combination thereof, and a third domain, which is an Fc region of modified immunoglobulin, coupled to the C-terminal of the first domain.

In some aspects, an IL-7 fusion protein that can be used with the present methods comprises an amino sequence that is at least 70%, at least 75%, at least 80%, at least 85%, at least 90%, at least 91%, at least 92%, at least 93%, at least 94%, at least 95%, at least 96%, at least 97%, at least 98%, at least 98%, or at least 99% identical to an amino acid sequence set forth in SEQ ID NOs: 21-25. In certain aspects, an IL-7 fusion protein of the present disclosure comprises the amino acid sequence set forth in SEQ ID NOs: 21-25. In certain aspects, an IL-7 fusion protein disclosed herein comprises the amino acid sequence set forth in SEQ ID NOs: 26 and 27.

In some aspects, an IL-7 protein useful for the present disclosure can increase absolute lymphocyte counts in a subject when administered to the subject. In certain aspects, the subject suffers from a disease or disorder described herein (e.g., cancer). In some aspects, the subject is a healthy individual (e.g., does not suffer from a disease or disorder described herein, e.g., cancer). In certain aspects, the absolute lymphocyte count is increased by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% or more, compared to a reference (e.g., corresponding level in a subject that did not receive the IL-7 protein).

### IV. Nucleotide Vaccines, Vectors, Host Cells

As described herein, in some aspects, methods disclosed herein comprise administering a nucleotide vaccine in combination with an IL-7 to a subject (e.g., suffering from a tumor). In some aspects, nucleotide vaccines can comprise one or more vectors that include one or more heterologous nucleic acids encoding a tumor antigen. In some aspects, the nucleotide vaccines can further comprise one or more vectors that include one or more heterologous nucleic acids encoding an additional agent, e.g., IL-7 protein disclosed herein. In certain aspects, the tumor antigen and the IL-7 protein can be encoded in a single vector. In some aspects, the tumor antigen and the IL-7 protein are encoded in separate vectors.

As will be apparent to those skilled in the arts, nucleotide vaccines disclosed herein can encode any antigen or protein known in the art that can be useful in treating a tumor (or cancer). As described herein, in some aspects, the nucleotide vaccine encodes a tumor antigen. Non-limiting examples of tumor antigens include Lrrc27, Plekho1, Pttg1, Xpo4, Exoc4, Pank3, Tmem101, Map3k6, Met, BC057079, Hist1h3e, Prkag1, Neil3, guanylate cyclase C (GC-C), epidermal growth factor receptor (EGFR or erbB-1), human epidermal growth factor receptor 2 (HER2 or erbf12), erbB-3, erbB-4, MUC-1, melanoma-associated chondroitin sulfate proteoglycan (MCSP), mesothelin (MSLN), folate receptor 1 (FOLR1), CD4, CD19, CD20, CD22, CD30, CD33, CD38, CD44, CD44v6, CD44v7/8, CD70, CD123, CD138, CD171, CEA, CSPG4, CXCR5, c-Met, HERV-envelope protein, eriostin, Bigh3, SPARC, BCR, CD79, CD37, EGFRvIII, EGP2, EGP40, IGFr, L1CAM, AXL, Tissue Factor (TF), CD74, EpCAM, EphA2, MRP3cadherin 19 (CDH19), epidermal growth factor 2 (HER2), 5T4, 8H9, αᵥβ₆ integrin, BCMA, B7-H3, B7-H6, CAIX, CA9, FAP, FBP, fetal AchR, FRcc, GD2, GD3, glypican-1 (GPC1), glypican-2 (GPC2), glypican-3 (GPC3), HLA-A1+MAGE1, HLA-A1+NY-ESO-1, IL-13Rcc2, Lewis-Y, KDR, MCSP, mesothelin, Muc1, Muc16, NCAM, NKG2D ligands, NY-ESO-1, PRAME, PSC1, PSCA, PSMA, ROR1, ROR2, SP17, surviving, TAG72, TEMs, carcinoembryonic antigen, HMW-MAA, VEGF, CLDN18.2, or combinations thereof. In some aspects, the tumor antigen comprises a cancer neoantigen (i.e., mutated antigens specifically expressed by tumor tissue and not expressed on the surface of normal cells). Examples of such antigens, including methods of identification, are known in the art. See, e.g., Hutchison et al., Mamm Genome 29(11): 714-730 (August 2018), which is incorporated herein by reference in its entirety.

In some aspects, nucleotides vaccines of the present disclosure encodes at least about one, at least about two, at least about three, at least about four, at least about five, at least about six, at least about seven, at least about eight, at least about nine, at least about ten, at least about 11, at least about 12, at least about 13, at least about 14, at least about 15, at least about 16, at least about 17, at least about 18, at least about 19, at least about 20, at least about 21, at least about 22, at least about 23, at least about 24, at least about 25, at least about 26, at least bout 27, at least about 28, at least about 29, at least about 30, at least about 35, at least about 40, at least about 45, or at least about 50 or more different tumor antigens.

As described herein, nucleotide vaccines of the present disclosure comprise both DNA vaccine and mRNA vaccine. DNA vaccines, including methods of making, are described, for example, in U.S. Pat. Nos. 7,795,017 B2 and 5,643,578 A, each of which is herein incorporated by reference in its entirety. mRNA vaccines, including methods of making, are described, for example in U.S. Publication Nos. 2018/0311336 A1 and 2020/0085852 A1, each of which is herein incorporated by reference in its entirety.

As is known in the art, a large number of factors can influence the efficiency of expression of antigen genes and/or the immunogenicity of nucleotide vaccines. Non-limiting examples of such factors include the reproducibility of inoculation, construction of the plasmid vector, choice of the promoter used to drive antigen gene expression and stability of the inserted gene in the plasmid. Depending on their origin, promoters differ in tissue specificity and efficiency in initiating mRNA synthesis (Xiang et al., Virology, 209:564-579 (1994); Chapman et al., Nucle. Acids. Res., 19:3979-3986 (1991)), which are incorporated herein by reference in their entirety. To date, most DNA vaccines in mammalian systems have relied upon viral promoters derived from cytomegalovirus (CMV). These have had good efficiency in both muscle and skin inoculation in a number of mammalian species. Another factor known to affect the immune response elicited by nucleotide vaccine immunization is the method of delivery. For instance, parenteral routes can yield low rates of gene transfer and produce considerable variability of gene expression (Montgomery et al., DNA Cell Bio., 12:777-783 (1993)). High-velocity inoculation of plasmids (e.g., DNA plasmids), using a gene-gun, have been shown to enhance immune responses in mice (Fynan et al., Proc. Natl. Acad. Sci., 90:11478-11482 (1993); Eisenbraun et al., DNA Cell Biol., 12: 791-797 (1993), which are incorporated herein by reference in their entirety), presumably because of a greater efficiency of DNA transfection and more effective antigen presentation by dendritic cells. Vectors containing the nucleotide vaccines of the present disclosure can also be introduced into the desired host by other methods known in the art, e.g., transfection, electroporation, microinjection, transduction, cell fusion, DEAE dextran, calcium phosphate precipitation, lipofection (lysosome fusion), or a DNA vector transporter (see, e.g., Wu et al., J. Biol. Chem. 267:963-967 (1992); Wu and Wu, J. Biol. Chem. 263:14621-14624 (1988); Hartmut et al., U.S. Pat. No. 5,792,645 A), each of which is incorporated herein by reference in its entirety.

In some aspects, the one or more vectors used in constructing the nucleotide vaccines of the present disclosure comprises expression vectors, viral vectors, plasmid vectors, or combinations thereof.

In some aspects, the vector is an expression vector. As used herein, an “expression vector” refers to any nucleic acid construct which contains the necessary elements for the transcription and translation of an inserted coding sequence, or in the case of an RNA viral vector, the necessary elements for replication and translation, when introduced into an appropriate host cell. Expression vectors can include plasmids, phagemids, viruses, and derivatives thereof. Once the expression vector is inside the cell, the protein that is encoded by the gene can be produced by the cellular-transcription and translation machinery ribosomal complexes. In some aspects, the plasmid can be engineered to contain regulatory sequences that act as enhancer and promoter regions and lead to efficient transcription of the gene carried on the expression vector.

In some aspects, the nucleotide vaccines described herein can comprise a circular plasmid or a linear nucleic acid. In some aspects, the circular plasmid and linear nucleic acid are capable of directing expression of a particular heterologous nucleotide sequence (e.g., encoding a tumor antigen and/or IL-7 protein described herein) in an appropriate subject cell. In some aspects, the vector can have a promoter operably linked to the nucleotide sequence (e.g., encoding a tumor antigen and/or IL-7 protein described herein), which can be operably linked to termination signals. In certain aspects, the vector can also contain sequences required for proper translation of the nucleotide sequence (e.g., encoding a tumor antigen and/or IL-7 protein described herein). In some aspects, the vector comprising the nucleotide sequence of interest (e.g., encoding a tumor antigen and/or IL-7 protein described herein) can be chimeric, meaning that at least one of its components is heterologous with respect to at least one of its other components. In some aspects, the expression of the nucleotide sequence (e.g., encoding a tumor antigen and/or IL-7 protein described herein) in the expression cassette can be under the control of a constitutive promoter or an inducible promoter, which initiates transcription only when the host cell is exposed to some particular external stimulus. In the case of a multicellular organism, the promoter can also be specific to a particular tissue or organ or stage of development.

In some aspects, the nucleotide vaccine comprises a circular plasmid, which can transform a target cell by integration into the cellular genome or exist extrachromosomally (e.g., autonomous replicating plasmid with an origin of replication). In such aspects, the vector can be pVAX, pcDNA3.0, provax, or any other expression vector capable of expressing a heterologous nucleic acid (e.g., encoding a tumor antigen and/or IL-7 protein described herein) and enabling a cell to translate the sequence such that is recognized by the immune system.

In some aspects, the nucleotide vaccine comprises a linear nucleic acid (e.g., encoding a tumor antigen and/or IL-7 protein described herein) that is capable of being efficiently delivered to a subject and expressing one or more desired proteins (e.g., tumor antigen and/or IL-7 protein disclosed herein). In certain aspects, the linear nucleic acid can contain a promoter, an intron, a stop codon, and/or a polyadenylation signal, which help regulate the expression of the desired proteins (e.g., tumor antigen and/or IL-7 protein disclosed herein). In certain aspects, the linear nucleic acid does not contain any antibiotic resistance genes and/or a phosphate backbone. In some aspects, the linear nucleic acid does not contain other nucleic acid sequences unrelated to the desired protein (e.g., tumor antigen and/or IL-7 protein disclosed herein) expression.

In some aspects, the linear nucleic acid (e.g., tumor antigen and/or IL-7 protein disclosed herein) can be derived from any plasmid capable of being linearized. Non-limiting examples of plasmids that can be used include pNP (Puerto Rico/34), pM2 (New Caledonia/99), WLV009, pVAX, pcDNA3.0, provax, or combinations thereof.

As described herein, in some aspects, vectors useful for constructing the nucleotide vaccines of the present disclosure can comprise a promoter. In some aspects, the promoter can be any promoter that is capable of driving gene expression and regulating expression of the nucleic acid (e.g., encoding a tumor antigen and/or IL-7 protein disclosed herein). In certain aspects, the promoter is a cis-acting sequence element required for transcription via a DNA dependent RNA polymerase. Selection of the promoter used to direct expression of a heterologous nucleic acid (e.g., encoding a tumor antigen and/or IL-7 protein disclosed herein) can depend on the particular application. In some aspects, the promoter can be a CMV promoter, SV40 early promoter, SV40 later promoter, metallothionein promoter, murine mammary tumor virus promoter, Rous sarcoma virus promoter, polyhedrin promoter, or another promoter shown effective for expression in eukaryotic cells. Non-limiting examples of promoters that are useful for the present disclosure are described in U.S. Pat. No. 7,557,200 B2, which is herein incorporated by reference in its entirety.

In some aspects, the vectors that can be used with the present disclosure include an enhancer and an intron with functional splice donor and acceptor sites. In certain aspects, the vector can contain a transcription termination region downstream of the structural gene to provide for efficient termination. The termination region can be obtained from the same gene as the promoter sequence or can be obtained from different genes.

In some aspects, the vector is a viral vector. As used herein, viral vectors include, but are not limited to, nucleic acid sequences from the following viruses: retrovirus, such as Moloney murine leukemia virus, Harvey murine sarcoma virus, murine mammary tumor virus, and Rous sarcoma virus; lentivirus; adenovirus; adeno-associated virus; SV40-type viruses; polyomaviruses; Epstein-Barr viruses; papilloma viruses; herpes virus; vaccinia virus; polio virus; and RNA virus such as a retrovirus. One can readily employ other vectors well-known in the art. Certain viral vectors are based on non-cytopathic eukaryotic viruses in which non-essential genes have been replaced with the gene of interest. Non-cytopathic viruses include retroviruses, the life cycle of which involves reverse transcription of genomic viral RNA into DNA with subsequent proviral integration into host cellular DNA.

In some aspects, a vector is derived from an adeno-associated virus. In some aspects, a vector is derived from a lentivirus. Examples of the lentiviral vectors are disclosed in WO9931251, WO9712622, WO9817815, WO9817816, and WO9818934, each which is incorporated herein by reference in its entirety.

Also encompassed by the present disclosure is a method for making a therapeutic agent disclosed herein (e.g., an IL-7 protein). In some aspects, such a method can comprise expressing the therapeutic agent (e.g., an IL-7 protein) in a cell comprising a nucleic acid molecule encoding the therapeutic agent, e.g., SEQ ID NOs: 29-39. Additional details regarding the method for making an IL-7 protein disclosed herein are provided, e.g., in U.S. Publ. No. 2018/0273596 A1, which is herein incorporated by reference in its entirety. Host cells comprising these nucleotide sequences are encompassed herein. Non-limiting examples of host cell that can be used include immortal hybridoma cell, NS/0 myeloma cell, 293 cell, Chinese hamster ovary (CHO) cell, HeLa cell, human amniotic fluid-derived cell (CapT cell), COS cell, or combinations thereof.

### V. Pharmaceutical Compositions

Further provided herein are compositions comprising one or more therapeutic agents (e.g., a nucleotide vaccine and/or IL-7) having the desired degree of purity in a physiologically acceptable carrier, excipient or stabilizer (Remington's Pharmaceutical Sciences (1990) Mack Publishing Co., Easton, PA). In some aspects, a composition disclosed herein comprises one or more nucleotide vaccines encoding a tumor antigen. In some aspects, a composition disclosed herein comprises an IL-7 (e.g., those disclosed herein). As disclosed herein, such compositions can be used in combination (e.g., a first composition comprising a nucleotide vaccine, and a second composition comprising an IL-7). In some of these aspects, the composition comprising an IL-7 is administered after administering the composition comprising the nucleotide vaccine (e.g., after the peak expansion phase of the tumor-specific T cell immune response). In certain aspects, a composition comprises both (i) a nucleotide vaccine encoding a tumor antigen, and (ii) an IL-7.

Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN©, PLURONICS© or polyethylene glycol (PEG).

In some aspects, a composition disclosed herein comprises one or more additional components selected from: a bulking agent, stabilizing agent, surfactant, buffering agent, or combinations thereof.

Buffering agents useful for the current disclosure can be a weak acid or base used to maintain the acidity (pH) of a solution near a chosen value after the addition of another acid or base. Suitable buffering agents can maximize the stability of the pharmaceutical compositions by maintaining pH control of the composition. Suitable buffering agents can also ensure physiological compatibility or optimize solubility. Rheology, viscosity and other properties can also dependent on the pH of the composition. Common buffering agents include, but are not limited to, a Tris buffer, a Tris-Cl buffer, a histidine buffer, a TAE buffer, a HEPES buffer, a TBE buffer, a sodium phosphate buffer, a MES buffer, an ammonium sulfate buffer, a potassium phosphate buffer, a potassium thiocyanate buffer, a succinate buffer, a tartrate buffer, a DIPSO buffer, a HEPPSO buffer, a POPSO buffer, a PIPES buffer, a PBS buffer, a MOPS buffer, an acetate buffer, a phosphate buffer, a cacodylate buffer, a glycine buffer, a sulfate buffer, an imidazole buffer, a guanidine hydrochloride buffer, a phosphate-citrate buffer, a borate buffer, a malonate buffer, a 3-picoline buffer, a 2-picoline buffer, a 4-picoline buffer, a 3,5-lutidine buffer, a 3,4-lutidine buffer, a 2,4-lutidine buffer, a Aces, a diethylmalonate buffer, a N-methylimidazole buffer, a 1,2-dimethylimidazole buffer, a TAPS buffer, a bis-Tris buffer, a L-arginine buffer, a lactate buffer, a glycolate buffer, or combinations thereof.

In some aspects, a composition disclosed herein further comprises a bulking agent. Bulking agents can be added to a pharmaceutical product in order to add volume and mass to the product, thereby facilitating precise metering and handling thereof. Bulking agents that can be used with the present disclosure include, but are not limited to, sodium chloride (NaCl), mannitol, glycine, alanine, or combinations thereof.

In some aspects, a composition disclosed herein can also comprise a stabilizing agent. Non-limiting examples of stabilizing agents that can be used with the present disclosure include: sucrose, trehalose, raffinose, arginine, or combinations thereof.

In some aspects, a composition disclosed herein comprises a surfactant. In certain aspects, the surfactant can be selected from the following: alkyl ethoxylate, nonylphenol ethoxylate, amine ethoxylate, polyethylene oxide, polypropylene oxide, fatty alcohols such as cetyl alcohol or oleyl alcohol, cocamide MEA, cocamide DEA, polysorbates, dodecyl dimethylamine oxide, or combinations thereof. In some aspects, the surfactant is polysorbate 20 or polysorbate 80.

In some aspects, a composition comprising a nucleotide vaccine can be formulated using the same formulation used for formulating a composition comprising an IL-7. In some aspects, a nucleotide vaccine and IL-7 are formulated using different formulations.

In some aspects, an IL-7 disclosed herein is formulated in a composition comprising (a) a basal buffer, (b) a sugar, and (c) a surfactant. In certain aspects, the basal buffer comprises histidine-acetate or sodium citrate. In some aspects, the basal buffer is at a concentration of about 10 to about 50 nM. In some aspects, a sugar comprises sucrose, trehalose, dextrose, or combinations thereof. In some aspects, the sugar is present at a concentration of about 2.5 to about 5.0 w/v %. In certain aspects, the surfactant is selected from polysorbate, polyoxyethylene alkyl ether, polyoxyethylene stearate, alkyl sulfates, polyvinyl pyridone, poloxamer, or combinations thereof. In some aspects, the surfactant is at a concentration of about 0.05% to about 6.0 w/v %.

In some aspects, a composition disclosed herein (e.g., comprising a nucleotide vaccine and/or IL-7) further comprises an amino acid. In certain aspects, the amino acid is selected from arginine, glutamate, glycine, histidine, or combinations thereof. In certain aspects, the composition further comprises a sugar alcohol. Non-limiting examples of sugar alcohol includes: sorbitol, xylitol, maltitol, mannitol, or combinations thereof.

In some aspects, an IL-7 disclosed herein is formulated in a composition comprising the following: (a) sodium citrate (e.g., about 20 mM), (b) sucrose (e.g., about 5%), (c) sorbitol (e.g., about 1.5%), and (d) Tween 80 (e.g., about 0.05%).

In some aspects, an IL-7 is formulated as described in U.S. Publ. No. 2018/0327472 A1, which is incorporated herein in its entirety.

A pharmaceutical composition disclosed herein can be formulated for any route of administration to a subject. Specific examples of routes of administration include intramuscularly, cutaneously, subcutaneously, ophthalmic, intravenously, intraperitoneally, intradermally, intraorbitally, intracerebrally, intracranially, intraspinally, intraventricular, intrathecally, intracistemally, intracapsularly, or intratumorally. Parenteral administration, characterized by, e.g., cutaneous, subcutaneous, intramuscular, or intravenous injection, is also contemplated herein. In some aspects, a nucleotide vaccine and IL-7 are administered using the same route of administration. In some aspects, a nucleotide vaccine and IL-7 are administered using different routes of administration.

Injectables can be prepared in conventional forms, either as liquid solutions or suspensions, solid forms suitable for solution or suspension in liquid prior to injection, or as emulsions. The injectables, solutions and emulsions also contain one or more excipients. Suitable excipients are, for example, water, saline, dextrose, glycerol or ethanol. In addition, if desired, the pharmaceutical compositions to be administered can also contain minor amounts of non-toxic auxiliary substances such as wetting or emulsifying agents, pH buffering agents, stabilizers, solubility enhancers, and other such agents, such as for example, sodium acetate, sorbitan monolaurate, triethanolamine oleate and cyclodextrins.

Pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances. Examples of aqueous vehicles include Sodium Chloride Injection, Ringers Injection, Isotonic Dextrose Injection, Sterile Water Injection, Dextrose and Lactated Ringers Injection. Nonaqueous parenteral vehicles include fixed oils of vegetable origin, cottonseed oil, corn oil, sesame oil and peanut oil. Antimicrobial agents in bacteriostatic or fungistatic concentrations can be added to parenteral preparations packaged in multiple-dose containers which include phenols or cresols, mercurials, benzyl alcohol, chlorobutanol, methyl and propyl p-hydroxybenzoic acid esters, thimerosal, benzalkonium chloride and benzethonium chloride. Isotonic agents include sodium chloride and dextrose. Buffers include phosphate and citrate. Antioxidants include sodium bisulfate. Local anesthetics include procaine hydrochloride. Suspending and dispersing agents include sodium carboxymethylcelluose, hydroxypropyl methylcellulose and polyvinylpyrrolidone. Emulsifying agents include Polysorbate 80 (TWEEN© 80). A sequestering or chelating agent of metal ions includes EDTA. Pharmaceutical carriers also include ethyl alcohol, polyethylene glycol and propylene glycol for water miscible vehicles; and sodium hydroxide, hydrochloric acid, citric acid or lactic acid for pH adjustment.

Preparations for parenteral administration include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions can be either aqueous or nonaqueous.

If administered intravenously, suitable carriers include physiological saline or phosphate buffered saline (PBS), and solutions containing thickening and solubilizing agents, such as glucose, polyethylene glycol, and polypropylene glycol and mixtures thereof.

Topical mixtures comprising an antibody are prepared as described for the local and systemic administration. The resulting mixture can be a solution, suspension, emulsions or the like and can be formulated as creams, gels, ointments, emulsions, solutions, elixirs, lotions, suspensions, tinctures, pastes, foams, aerosols, irrigations, sprays, suppositories, bandages, dermal patches or any other formulations suitable for topical administration.

A therapeutic agent described herein can be formulated as an aerosol for topical application, such as by inhalation (see, e.g., U.S. Pat. Nos. 4,044,126, 4,414,209 and 4,364,923, which describe aerosols for delivery of a steroid useful for treatment of inflammatory diseases, particularly asthma). These formulations for administration to the respiratory tract can be in the form of an aerosol or solution for a nebulizer, or as a microfine powder for insufflations, alone or in combination with an inert carrier such as lactose. In such a case, the particles of the formulation can have diameters of less than about 50 microns, e.g., less than about 10 microns.

A therapeutic agent disclosed herein can be formulated for local or topical application, such as for topical application to the skin and mucous membranes, such as in the eye, in the form of gels, creams, and lotions and for application to the eye or for intracisternal or intraspinal application. Topical administration is contemplated for transdermal delivery and also for administration to the eyes or mucosa, or for inhalation therapies. Nasal solutions of the antibody alone or in combination with other pharmaceutically acceptable excipients can also be administered.

Transdermal patches, including iontophoretic and electrophoretic devices, are well known to those of skill in the art, and can be used to administer a therapeutic agent (e.g., those disclosed herein). For example, such patches are disclosed in U.S. Pat. Nos. 6,267,983, 6,261,595, 6,256,533, 6,167,301, 6,024,975, 6,010,715, 5,985,317, 5,983,134, 5,948,433, and 5,860,957, each of which is herein incorporated by reference in its entirety.

In certain aspects, a pharmaceutical composition comprising a therapeutic agent described herein is a lyophilized powder, which can be reconstituted for administration as solutions, emulsions and other mixtures. It can also be reconstituted and formulated as solids or gels. The lyophilized powder is prepared by dissolving an antibody or antigen-binding portion thereof described herein, or a pharmaceutically acceptable derivative thereof, in a suitable solvent. In some aspects, the lyophilized powder is sterile. The solvent can contain an excipient which improves the stability or other pharmacological component of the powder or reconstituted solution, prepared from the powder. Excipients that can be used include, but are not limited to, dextrose, sorbitol, fructose, corn syrup, xylitol, glycerin, glucose, sucrose or other suitable agent. The solvent can also contain a buffer, such as citrate, sodium or potassium phosphate or other such buffer known to those of skill in the art at, in some aspects, about neutral pH. Subsequent sterile filtration of the solution followed by lyophilization under standard conditions known to those of skill in the art provides the desired formulation. In some aspects, the resulting solution can be apportioned into vials for lyophilization. Each vial can contain a single dosage or multiple dosages of the compound. The lyophilized powder can be stored under appropriate conditions, such as at about 4° C. to room temperature.

Reconstitution of this lyophilized powder with water for injection provides a formulation for use in parenteral administration. For reconstitution, the lyophilized powder is added to sterile water or other suitable carrier. The precise amount depends upon the selected compound. Such amount can be empirically determined.

Compositions provided herein can also be formulated to be targeted to a particular tissue, receptor, or other area of the body of the subject to be treated. Many such targeting methods are well known to those of skill in the art. All such targeting methods are contemplated herein for use in the instant compositions. For non-limiting examples of targeting methods, see, e.g., U.S. Pat. Nos. 6,316,652, 6,274,552, 6,271,359, 6,253,872, 6,139,865, 6,131,570, 6,120,751, 6,071,495, 6,060,082, 6,048,736, 6,039,975, 6,004,534, 5,985,307, 5,972,366, 5,900,252, 5,840,674, 5,759,542 and 5,709,874, each of which is incorporated herein by reference in its entirety.

The compositions to be used for in vivo administration can be sterile. This is readily accomplished by filtration through, e.g., sterile filtration membranes.

The following examples are merely illustrative and should not be construed as limiting the scope of this disclosure in any way as many variations and equivalents will become apparent to those skilled in the art upon reading the present disclosure

### EXAMPLES

### Example 1: Analysis of Administration Schedule of IL-7 and Nucleotide Vaccine Combination Therapy

To begin assessing the effect of IL-7 administration on the efficacy of a nucleotide vaccine in treating a tumor (or cancer), DNA vaccines encoding one or more neoantigen epitopes from an estrogen-receptor positive murine breast cancer was constructed. Briefly, nucleic acids encoding one or more of the following epitopes were inserted into a single pcDNA3.1(+) backbone: Lrrc27, Plekho1, Pttg1, Xpo4, Exoc4, Pank3, Tmem101, Map3k6, Met, BC057079, Hist1h3e, Prkag1, and Neil3. See Hundal et al., Genome Med 8(1): 11 (January 2016), which is herein incorporated by reference in its entirety.

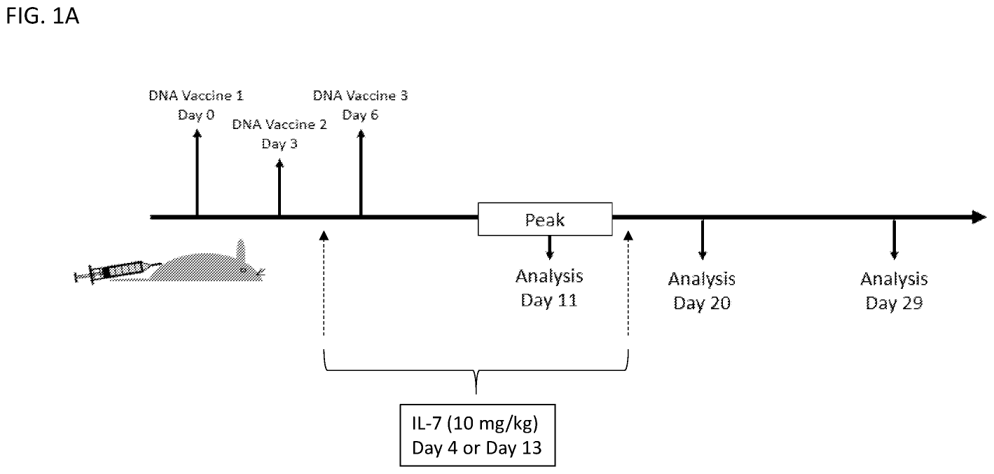

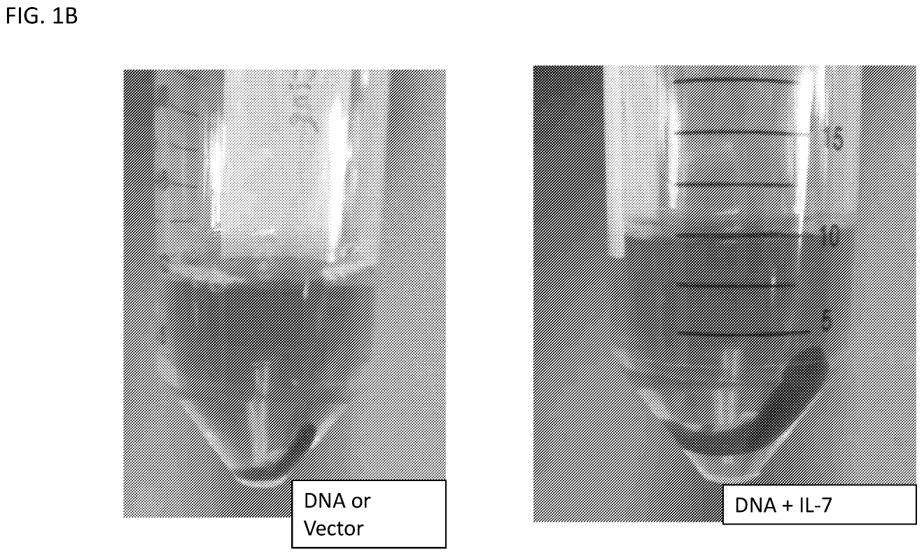

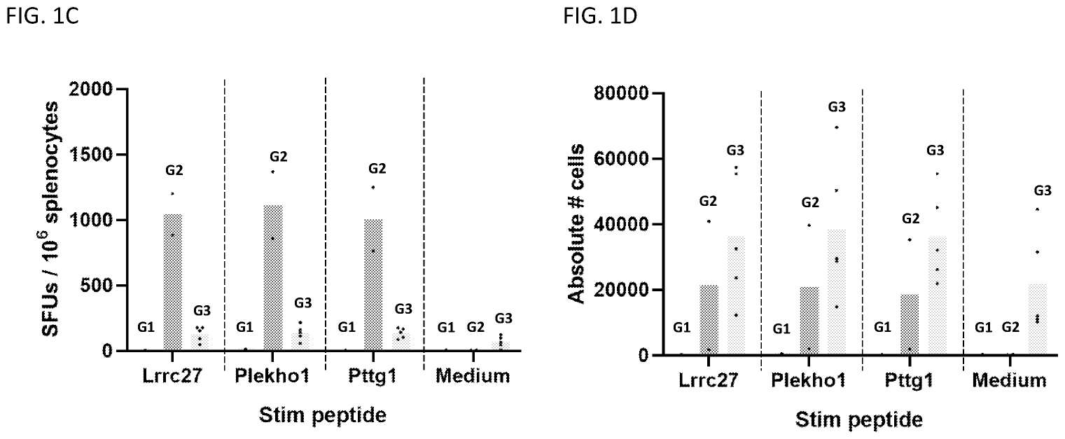

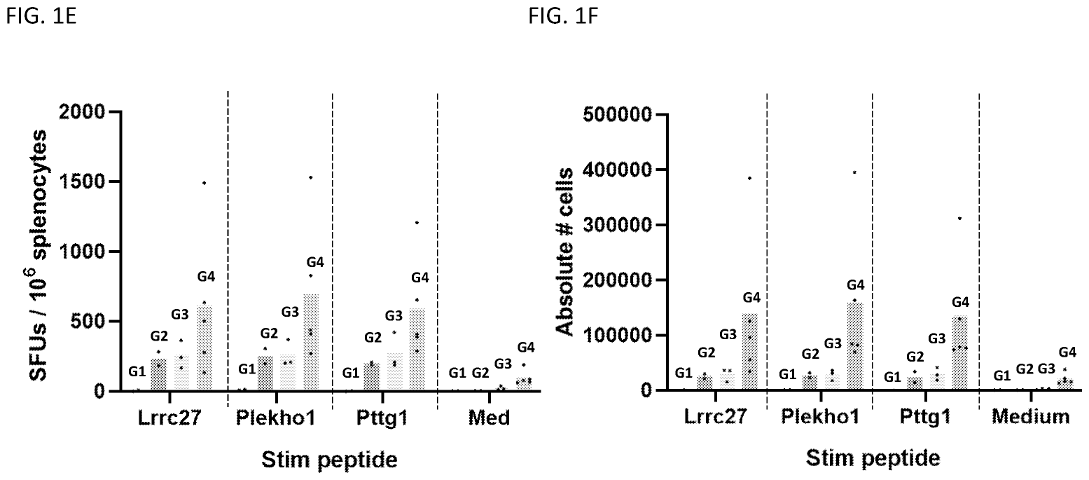

Then, naïve C57BL6 mice were immunized with the DNA vaccine construct for a total of three doses (4 μg/dose) at a dosing frequency of once every three days. See FIG. 1A. The DNA vaccine was administered cutaneously to the mice using a gene gun. Some of the animals received a single dose of IL-7 (10 mg/kg) at either day 4 or day 13 post initial DNA vaccine administration. The different treatment groups were as follows: (i) control vector only (G1); (ii) DNA vaccine only (G2); (iii) DNA vaccine+IL-7 administration at day 4 post initial DNA vaccine administration (G3); and (iv) DNA vaccine+IL-7 administration at day 13 post initial DNA vaccine administration (G4). Some of the animals from each of the treatment groups were sacrificed at days 11, 20, and 29 post initial DNA vaccine administration, and the tumor-specific T cell immune response was assessed in the spleen using an IFN-7 ELISPOT assay.

As shown in FIG. 1C, compared to animals treated with DNA vaccine alone (G2), administration of IL-7 at day 4 post initial DNA vaccine administration (G3) (i.e., expansion phase of the tumor-specific T cell immune response) did not result in a noticeable increase in the frequency of tumor-specific T cells (i.e., specific to Lrrc27, Plekho1, or Pttg1 epitopes) in the spleen. However, as shown in FIG. 1B, animals treated with IL-7 in combination with the DNA vaccine exhibited splenomegaly, resulting in a greater number of total splenocytes. Accordingly, the total number of tumor-specific T cells in the spleen of animals treated with IL-7 during the expansion phase was higher compared to animals that received the DNA vaccine alone (see FIG. 1D). However, by day 20 post initial DNA vaccine administration, there was no significant different in the tumor-specific T cell immune response (both in terms of frequency and total number) compared to animals treated with DNA vaccine alone (compare G2 and G3 in FIGS. 1E and 1F).

In animals treated with IL-7 during the contraction phase of the tumor-specific T cell immune response (i.e., at day 13 post initial DNA vaccine administration), there was a significant increase in both the frequency and total number of tumor-specific T cells in the spleen compared to the other treatment groups (see FIGS. 1E and 1F, respectively).

The above results demonstrate that the administration of IL-7 during T cell contraction can prolong the tumor-specific T cell immune response, which could be useful in treating a tumor (or cancer).

### Example 2: Analysis of the Dosage Effect of IL-7 on the Efficacy of IL-7 and Nucleotide Vaccine Combination Therapy

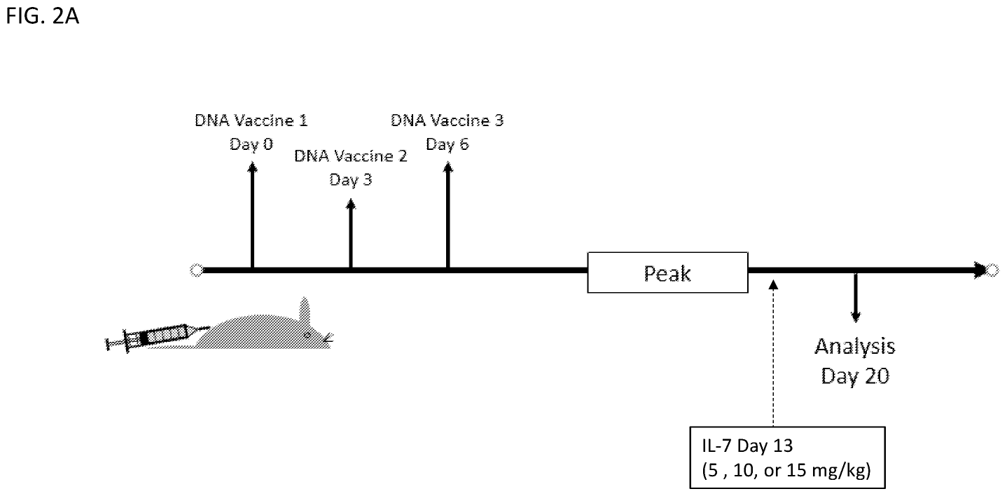

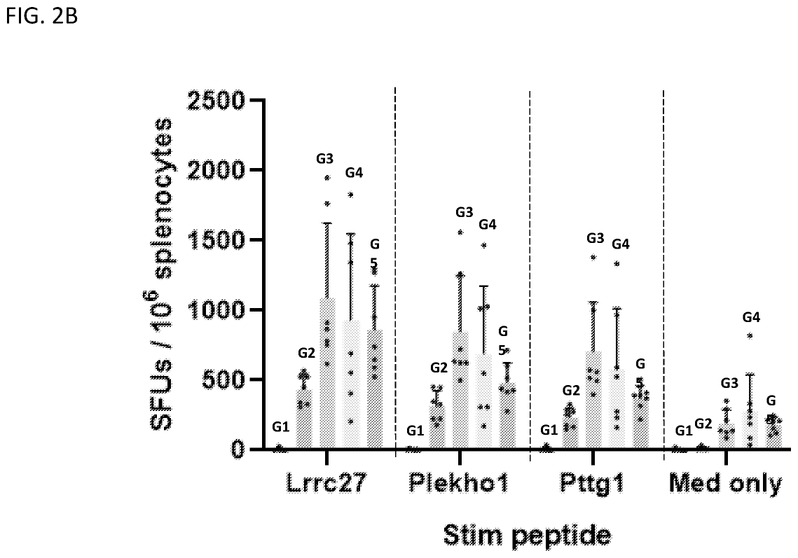

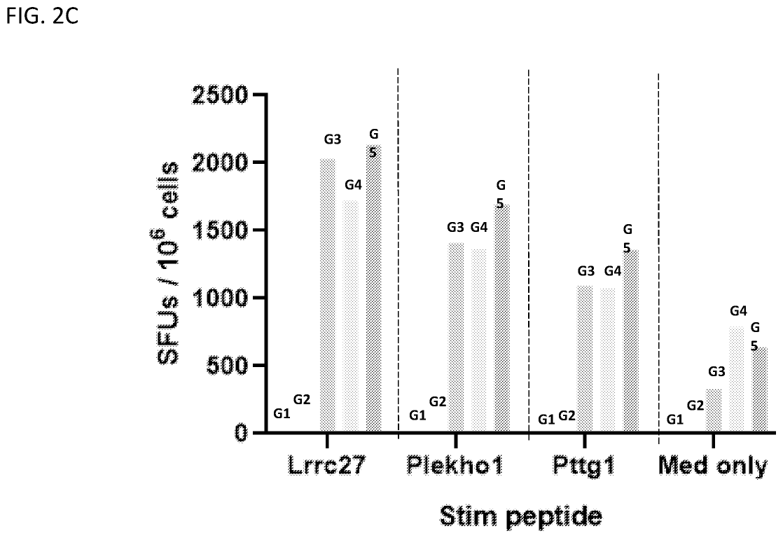

To identify the optimal dosage of IL-7 when administered in combination with a nucleotide vaccine disclosed herein, naïve C57BL6 mice were immunized with the DNA vaccine construct as described in Example 1 (see FIG. 2A). Then, at day 13 post initial DNA vaccine administration (i.e., during the contraction phase), a single dose of IL-7 was administered to the animals at one of the following doses: 5, 10, or 15 mg/kg. The different treatment groups were as follows: (i) control vector only (G1); (ii) DNA vaccine only (G2); (iii) DNA vaccine+5 mg/kg of IL-7 (G3); (iv) DNA vaccine+10 mg/kg of IL-7 (G4); and (v) DNA vaccine+15 mg/kg of IL-7 (G5). Animals were sacrificed at day 20 post initial DNA vaccine administration, and the tumor-specific T cell immune response was assessed in the spleen and the lymph nodes using an IFN-7 ELISPOT assay.

As shown in FIG. 2B, in agreement with the earlier data (see Example 1), animals treated with 10 mg/kg of IL-7 during the contraction phase had higher frequency of tumor-specific T cells (i.e., specific to Lrrc27, Plekho1, or Pttg1 epitopes) in the spleen compared to animals treated with DNA vaccine alone. Animals treated other dosages of IL-7 (i.e., 5 mg/kg or 15 mg/kg) during the contraction phase also had higher frequency of tumor-specific T cells compared to the DNA vaccine only group. While the differences did not appear to be statistically significant, the frequency of tumor-specific T cells in the spleen appeared to be moderately greater in animals treated with 5 mg/kg of IL-7. Similar results were observed in the lymph nodes (see FIG. 2C).

The above data further demonstrate the therapeutic effects of administering IL-7 during the contraction phase of a T cell immune response after DNA vaccine administration. The results additionally provide that the IL-7 can be administered at a dose of about 5 mg/kg to about 15 mg/kg

### Example 3: Analysis of the Anti-Tumor Effects of a Nucleotide Vaccine and IL-7 Combination Therapy

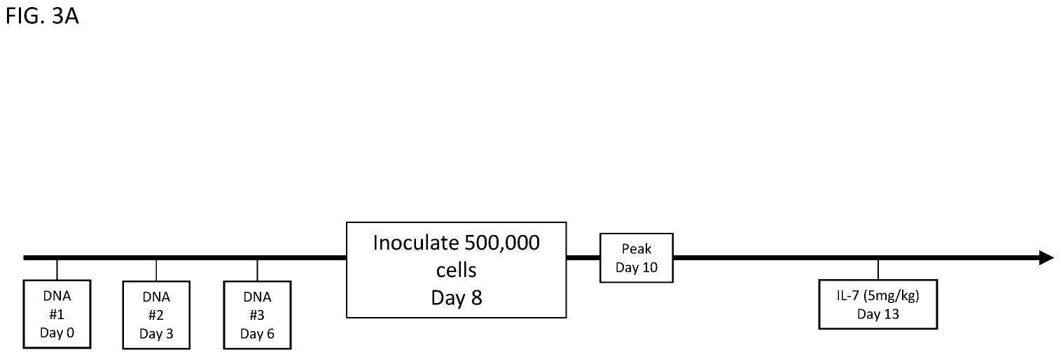

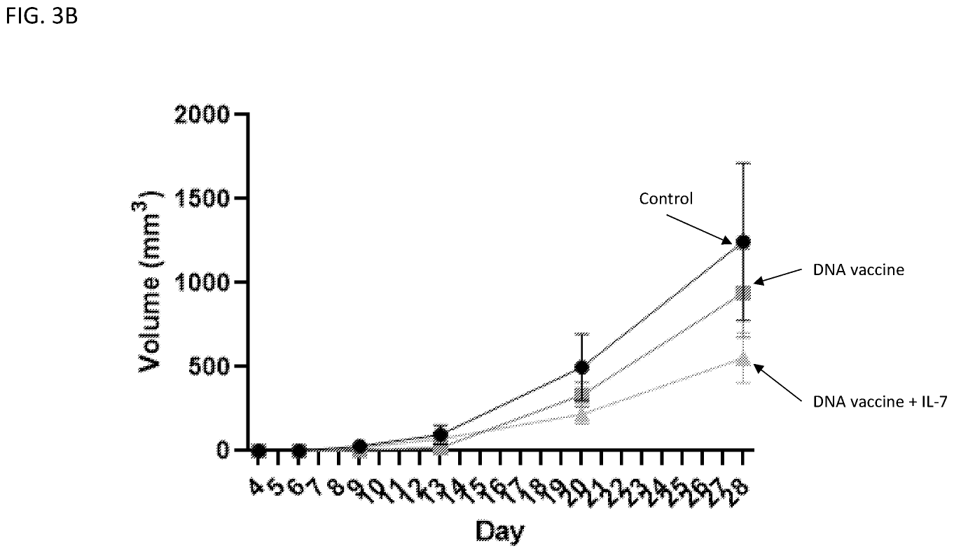

To assess the ability of the DNA vaccine and IL-7 combination therapy disclosed herein, a syngenic breast cancer animal model (E0771) was used. Briefly, the animals were immunized with the DNA vaccine construct as described in Examples 1 and 2 (see FIG. 3A). At day 8 post initial DNA vaccine administration, the animals were implanted subcutaneously with E0771 tumor cells (5×10⁵ cells/mouse). Then, at day 13 post initial DNA vaccine administration, some of the animals received a single administration of IL-7 (5 mg/kg). The treatment groups were as follows: (i) control vector alone (G1); (ii) DNA vaccine alone (G2); and (iii) DNA vaccine+IL-7 (5 mg/kg) at day 13 post initial DNA vaccine administration. Then, tumor volume was measured in the animals periodically.

As shown in FIG. 3B, compared to the control animals (i.e., G1), animals that received DNA vaccine alone had slightly lower tumor volume by the end of the experiment (i.e., day 28 post initial DNA vaccine administration). In animals that additionally received IL-7 during the contraction phase, the tumor volume was even more reduced.

These results demonstrate that the combination therapy disclosed herein (i.e., administering a nucleotide vaccine in combination with IL-7, wherein the IL-7 is administered after the peak expansion phase of the tumor-specific T cell immune response) can be useful in treating a tumor (or a cancer).

### Example 4: Analysis of the Effect of IL-7 Administration on Memory T Cells Induced after Nucleotide Vaccine Administration

To evaluate the effects of DNA vaccine and IL-7 combination therapy disclosed herein on neoantigen-specific memory T cell responses, an ovalbumin (OVA) animal model will be used. Ovalbumin is a model antigen and it is well-documented that vaccination with a DNA-based OVA vaccine induces a detectable memory response. The animals will receive one of the following treatments: (i) no treatment; (ii) DNA-based OVA vaccine alone; (iii) DNA vaccine encoding a tumor antigen (e.g., such as those described in Example 1) alone; (iv) DNA vaccine encoding a tumor antigen+IL-7. In some aspects, the IL-7 will be administered to the animals at a single dose of 5 mg/kg during the contraction phase of the tumor-specific T cell immune response (e.g., at day 13 post initial DNA vaccine administration).

At about day 60 post initial DNA vaccine administration, the mice will be sacrificed and mononuclear cells from the periphery (spleen, blood) and the bone marrow will be collected for immune monitoring. PBMCs isolated from the spleen and blood, following a 48-hour ex vivo antigen challenge, will be profiled with flow cytometry using antibody panels identifying the following T-cell populations (CD3⁺): cytotoxic T-cells (CD8⁺, CD11a/b⁺, IFNγ⁺), TH1 cells (CD4⁺, CD69, IFNγ⁺), memory T-cells (CD44⁺, CD62L⁺), and regulatory T-cells (CD4⁺, CD25⁺). Additionally, the proliferation of neoantigen-specific T cells will also be assessed with CFSE proliferation assay.

### Example 5: Analysis of the Effect of IL-7 Administration on T Cell-Mediated Cytotoxicity after Nucleotide Vaccine Administration

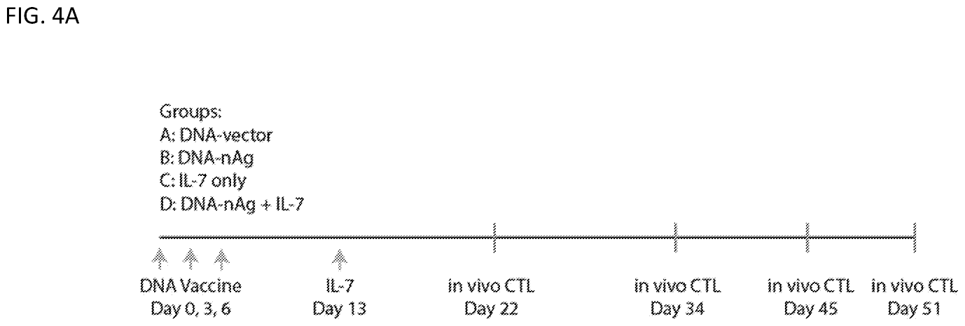

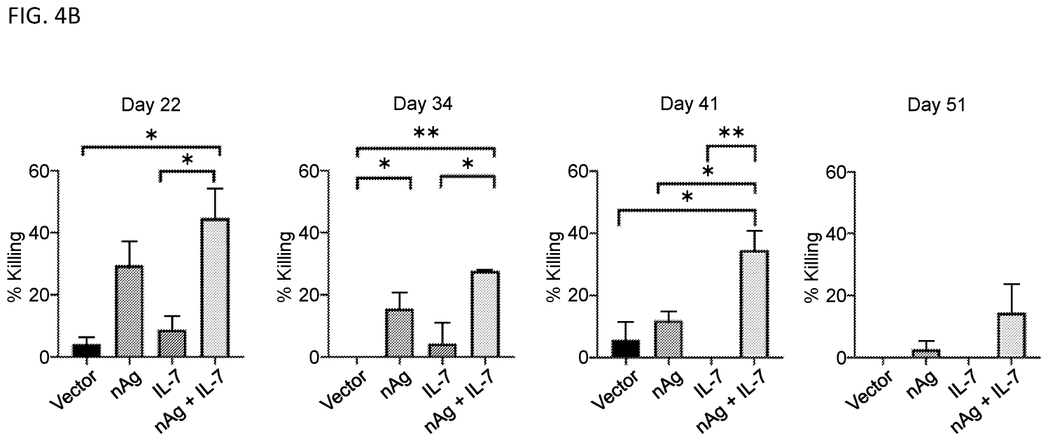

To further assess the therapeutic effects of the DNA vaccine and IL-7 combination therapy described herein, naïve mice were immunized with the DNA vaccine construct as described in Examples 1 and 2 (see FIG. 4A). At day 13 post initial immunization (i.e., during the contraction phase), some of the animals received a single administration of the IL-7 protein (5 mg/kg). The different treatment groups were as follows: (i) control vector only (G1); (ii) DNA vaccine only (G2); (iii) IL-7 protein alone (G3); and (iv) DNA vaccine+IL-7 protein (G4). At days 22, 34, 45, or 51 post initial immunization, the animals were intravenously injected with CFSE-labeled naïve splenocytes that were either unpulsed or pulsed with the neoantigens. Then, 24 hours later, animals from the different treatment groups were sacrificed and percent killing of the pulsed splenocytes was assessed by measuring CFSE expression via flow cytometry.

As shown in FIG. 4B, compared to animals that were treated with the control vector (G1) and IL-7 protein alone (G3), animals that were immunized with the DNA vaccine construct (described in Example 1) alone (G2) exhibited increased T cell-mediated killing. However, the greatest killing of the neoantigen-pulsed splenocytes was observed in animals that were treated with the combination therapy of DNA vaccine and IL-7 protein (G4). This was generally true for all four time points analyzed.

The above results confirm that the DNA vaccine and IL-7 combination treatment regimen described herein can induce potent cytotoxic T cells, which can be useful in the treatment of various cancers.

### Example 6: Further Analysis of Anti-Tumor Effects of a Nucleotide Vaccine and IL-7 Combination Therapy

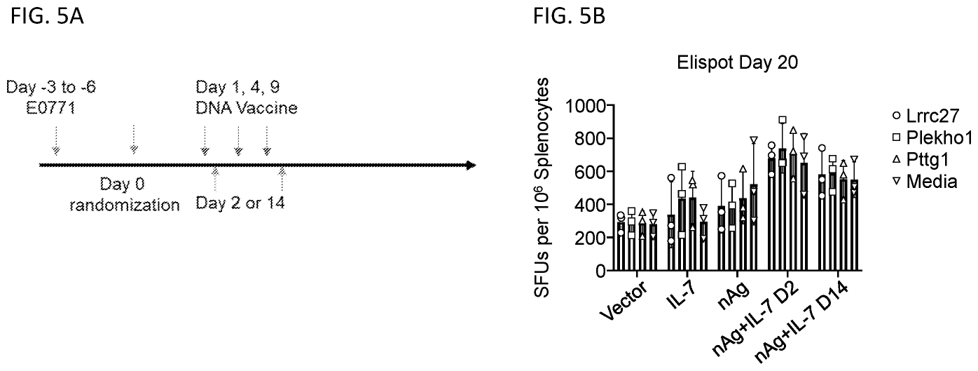

Further to the anti-tumor data provided in Example 3, it was next assessed whether DNA vaccine and IL-7 combination therapy described herein can also have therapeutic effects when administered after tumor induction. Briefly, as shown in FIG. 5A, mice were implanted subcutaneously with E0771 tumor cells in each flank (see, e.g., Example 3). Once palpable tumor size was reached (between about 25-100 mm³; approximately 3-6 days after tumor implantation), the animals were randomized (i.e., day 0). Then, at days 1, 4, and 9 post-randomization, some of the animals were immunized with the control vector or the DNA vaccine construct described in Examples 1 and 2. At day 2 or 14 post-randomization, some of the animals were intravenously treated with the IL-7 protein (5 mg/kg). The treatment groups were as follows: (i) control vector alone (“vector”); (ii) IL-7 protein alone (“IL-7”); (iii) DNA vaccine alone (“nAg”); (iv) DNA vaccine+IL-7, where IL-7 protein was administered at day 2 post-randomization (“nAg+IL-7 D2”); and (v) DNA vaccine+IL-7, where IL-7 protein was administered at day 14 post-randomization (“nAg+IL-7 D14”). At days 20 and 30 post-randomization, some of the animals from each of the treatment groups were sacrificed, and tumor-specific T cell immune response was assessed in the spleen using an IFN-7 ELISPOT assay.

As shown in FIG. 5B, at day 20 post-randomization, tumor animals that were treated with the combination therapy (DNA vaccine+IL-7) generally had greater tumor-specific T cell immune responses compared to animals from the different treatment groups. The increased T cell immune response was observed for all three epitopes assessed: Lrrc27, Plekho1, and Pttg1.

The above results further demonstrate the anti-tumor effects of the DNA vaccine+IL-7 combination therapy described herein, and suggests that administering such a therapy after tumor onset can also have therapeutic effects.

### Example 7: Analysis of a Nucleotide Vaccine and IL-7 Combination Therapy as a Prophylactic Cancer Vaccine

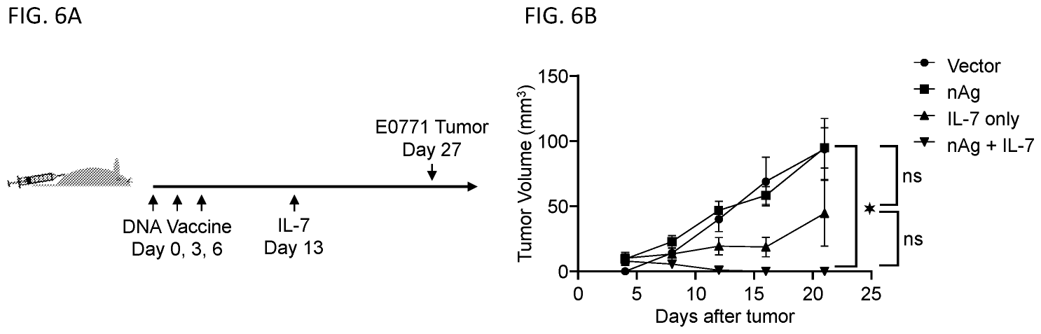

To assess whether the DNA vaccine and IL-7 combination therapy described herein could be used prophylactically, naïve mice were immunized with the control vector or the DNA vaccine construct as described in Examples 1 and 2. Specifically, the mice received the DNA vaccine for a total of three doses (4 μg/dose) at a dosing frequency of once every three days (i.e., days 0, 3, and 6). See FIG. 6A. At day 13 post initial DNA vaccine administration, some of the animals received a single intravenous dose of the IL-7 protein (5 mg/kg). The treatment groups were as follows: (i) control vector alone (“vector”); (ii) DNA vaccine alone (“nAg”); (iii) IL-7 protein alone (“IL-7 only”); and (iv) DNA vaccine and IL-7 (“nAg+IL-7”). At day 27 post initial DNA vaccine administration, animals from the different treatment groups were implanted subcutaneously with E0771 tumor cells in each flank (see, e.g., Example 3). At various times post tumor implantation, tumor volume was assessed in the animals.

As shown in FIG. 6B, animals treated with either the control vector alone or the DNA vaccine alone failed to control the growth of the tumor. However, in animals treated with the combination of DNA vaccine and IL-7, there was nearly no tumor growth observed. And, in animals treated with the IL-7 protein, there was also reduced tumor growth, at least as compared to the control vector or DNA vaccine only treated animals.

The above results demonstrate that the combination therapy described herein could also be used as a prophylactic vaccine in preventing and/or minimizing tumor growth. The data further demonstrate that IL-7 protein alone could also help in preventing and/or minimizing tumor growth in certain scenarios.

1. A method of treating a tumor in a subject in need thereof, comprising administering to the subject (1) a nucleotide vaccine encoding a tumor antigen and (2) an interleukin-7 (IL-7), wherein the administration of the nucleotide vaccine induces a tumor-specific T cell immune response, and wherein the IL-7 is administered to the subject within about 14 days of the nucleotide vaccine administration.

2. The method of claim 1, wherein the IL-7 is administered at about 14 days, at about 13 days, at about 12 days, at about 11 days, at about 10 days, at about nine days, at about eight days, at about seven days, at about six days, at about five days, at about four days, at about three days, at about two days, or at about one day after the nucleotide vaccine administration.

3. (canceled)

4. The method of claim 1, wherein the IL-7 is administered to the subject after a peak expansion phase of the tumor-specific T cell immune response.

5-6. (canceled)

7. A method of preventing or reducing the occurrence of a tumor in a subject in need thereof, comprising administering to the subject (1) a nucleotide vaccine encoding a tumor antigen and (2) an interleukin-7 (IL-7), wherein the administration of the nucleotide vaccine induces a tumor-specific T cell immune response, and wherein the nucleotide vaccine, the IL-7, or both the nucleotide vaccine and the IL-7 are administered to the subject prior to the occurrence of the tumor.

8. The method of claim 7, wherein the IL-7 is administered to the subject within about 14 days of the nucleotide vaccine administration.

9-10. (canceled)

11. A method of enhancing a tumor-specific T cell immune response in a subject in need thereof, comprising administering to the subject (1) a nucleotide vaccine encoding a tumor antigen and (2) an interleukin-7 (IL-7), wherein the administration of the nucleotide vaccine induces a tumor-specific T cell immune response, and wherein the IL-7 is administered to the subject within about 14 days of the nucleotide vaccine administration.

12-14. (canceled)

15. The method of claim 11, wherein the enhancing comprises (a) increasing a survival of tumor-specific T cells during a contraction phase of the tumor-specific T cell immune response, compared to a reference (e.g., corresponding value in a subject that received either IL-7 alone or nucleotide vaccine alone); (b) increasing a number of tumor-specific T cells during a contraction phase of the tumor-specific T cell immune response, compared to a reference (e.g., corresponding value in a subject that received either IL-7 alone or nucleotide vaccine alone); (c) expanding a T-cell receptor (TCR) repertoire of the tumor-specific T cell immune response, compared to a reference (e.g., corresponding value in a subject that received either IL-7 alone or nucleotide vaccine alone): (d) increasing a T cell immune response against a subdominant epitope of a tumor antigen, compared to a reference (e.g., corresponding value in a subject that received either IL-7 alone or nucleotide vaccine alone): (e) increasing the number of epitopes against which the tumor-specific T cell immune response is induced, compared to a reference (e.g., corresponding value in a subject that received either IL-7 alone or nucleotide vaccine alone): or (f) any combination of (a) to (e).

16-24. (canceled)

25. The method of claim 1, wherein the tumor antigen is derived from a breast cancer and the epitopes are selected from Lrrc27, Plekho1, Pttg1, Xpo4, Exoc4, Pank3, Tmem101, Map3k6, Met, BC057079, Hist1h3e, Prkag1, Neil3, or combinations thereof.

26-35. (canceled)

36. The method of claim 1, wherein the IL-7 is administered at a dose between about 5 mg/kg and about 15 mg/kg, between about 20 Ig/kg and about 600 μg/kg, or between about 600 μg/kg and about 2,000 μg/kg.

37-43. (canceled)

44. The method of claim 1, wherein the IL-7 is administered at a dosing frequency of about once a week, about once in two weeks, about once in three weeks, about once in four weeks, about once in five weeks, about once in six weeks, about once in seven weeks, about once in eight weeks, about once in nine weeks, about once in 10 weeks, about once in 11 weeks, or about once in 12 weeks.

45. The method of claim 1, wherein: (a) the nucleotide vaccine comprises a DNA vaccine, mRNA vaccine, or both: (b) the IL-7 is administered as a protein (IL-7 protein), nucleic acid encoding the IL-7 protein, or both: or (c) both (a) and (b).

46-49. (canceled)

50. The method of claim 1, wherein the IL-7 protein is administered as a fusion protein, and wherein the fusion protein comprises one or additional moieties that are conjugated to the IL-7 protein.

51. The method of claim 50, wherein the one or more additional moieties comprise: (a) an oligopeptide consisting of 1 to 10 amino acid residues: (b) a half-life extending moiety; or (c) both (a) and (b).

52. The method of claim 51, wherein; (a) the oligopeptide comprises methionine (M), glycine (G), methionine-methionine (MM), glycine-glycine (GG), methionine-glycine (MG), glycine-methionine (GM), methionine-methionine-methionine (MMM), methionine-methionine-glycine (MMG), methionine-glycine-methionine (MGM), glycine-methionine-methionine (GMM), methionine-glycine-glycine (MGG), glycine-methionine-glycine (GMG), glycine-glycine-methionine (GGM), glycine-glycine-glycine (GGG), methionine-glycine-glycine-methionine (MGGM) (SEQ ID NO: 41), methionine-methionine-glycine-glycine (MMGG) (SEQ ID NO: 42), glycine-glycine-methionine-methionine (GGMM) (SEQ ID NO: 43), methionine-glycine-methionine-glycine (MGMG) (SEQ ID NO: 44), glycine-methionine-methionine-glycine (GMMG) (SEQ ID NO: 45), glycine-glycine-glycine-methionine (GGGM) (SEQ ID NO: 46), methionine-glycine-glycine-glycine (MGGG) (SEQ ID NO: 47), glycine-methionine-glycine-glycine (GMGG) (SEQ ID NO: 48), glycine-glycine-methionine-glycine (GGMG) (SEQ ID NO: 49), glycine-glycine-methionine-methionine-methionine (GGMMM) (SEQ ID NO: 50), glycine-glycine-glycine-methionine-methionine (GGGMM) (SEQ ID NO: 51), glycine-glycine-glycine-glycine-methionine (GGGGM) (SEQ ID NO: 52), methionine-glycine-methionine-methionine-methionine (MGMMM) (SEQ ID NO: 53), methionine-glycine-glycine-methionine-methionine (MGGMM) (SEQ ID NO: 54), methionine-glycine-glycine-glycine-methionine (MGGGM) (SEQ ID NO: 55), methionine-methionine-glycine-methionine-methionine (MMGMM) (SEQ ID NO: 56), methionine-methionine-glycine-glycine-methionine (MMGGM) (SEQ ID NO: 57), methionine-methionine-glycine-glycine-glycine (MMGGG) (SEQ ID NO: 58), methionine-methionine-methionine-glycine-methionine (MMMGM) (SEQ ID NO: 59), methionine-glycine-methionine-glycine-methionine (MGMGM) (SEQ ID NO: 60), glycine-methionine-glycine-methionine-glycine (GMGMG) (SEQ ID NO: 61), glycine-methionine-methionine-methionine-glycine (GMMMG) (SEQ ID NO: 62), glycine-glycine-methionine-glycine-methionine (GGMGM) (SEQ ID NO: 63), glycine-glycine-methionine-methionine-glycine (GGMMG) (SEQ ID NO: 64), glycine-methionine-methionine-glycine-methionine (GMMGM) (SEQ ID NO: 65), methionine-glycine-methionine-methionine-glycine (MGMMG) (SEQ ID NO: 66), glycine-methionine-glycine-glycine-methionine (GMGGM) (SEQ ID NO: 67), methionine-methionine-glycine-methionine-glycine (MMGMG) (SEQ ID NO: 68), glycine-methionine-methionine-glycine-glycine (GMMGG) (SEQ ID NO: 69), glycine-methionine-glycine-glycine-glycine (GMGGG) (SEQ ID NO: 70), glycine-glycine-methionine-glycine-glycine (GGMGG) (SEQ ID NO: 71), glycine-glycine-glycine-glycine-glycine (GGGGG) (SEQ ID NO: 72), or combinations thereof; (b) the half-life extending moiety comprises an Fc, albumin, an albumin-binding polypeptide, Pro/Ala/Ser (PAS), a C-terminal peptide (CTP) of the R subunit of human chorionic gonadotropin, polyethylene glycol (PEG), long unstructured hydrophilic sequences of amino acids (XTEN), hydroxyethyl starch (HES), an albumin-binding small molecule, or a combination thereof; or (c) both (a) and (b).

53-57. (canceled)

58. The method of claim 1, wherein the IL-7 protein comprises an amino acid sequence having a sequence identity of at least about 70% to SEQ ID NOs: 1-6 and 15-25.

59. The method of claim 1, wherein the IL-7 and/or the nucleotide vaccine is administered to the subject parenthetically, intramuscularly, subcutaneously, ophthalmic, intravenously, intraperitoneally, intradermally, intraorbitally, intracerebrally, intracranially, intraspinally, intraventricular, intrathecally, intracistemally, intracapsularly, intratumorally, or any combination thereof.

60-61. (canceled)

62. The method of claim 1, wherein the tumor antigen comprises guanylate cyclase C (GC-C), epidermal growth factor receptor (EGFR or erbB-1), human epidermal growth factor receptor 2 (HER2 or erbB2), erbB-3, erbB-4, MUC-1, melanoma-associated chondroitin sulfate proteoglycan (MCSP), mesothelin (MSLN), folate receptor 1 (FOLR1), CD4, CD19, CD20, CD22, CD30, CD33, CD38, CD44, CD44v6, CD44v7/8, CD70, CD123, CD138, CD171, CEA, CSPG4, CXCR5, c-Met, HERV-envelope protein, eriostin, Bigh3, SPARC, BCR, CD79, CD37, EGFRvIII, EGP2, EGP40, IGFr, L1CAM, AXL, Tissue Factor (TF), CD74, EpCAM, EphA2, MRP3cadherin 19 (CDH19), epidermal growth factor 2 (HER2), 5T4, 8H9, αᵥβ₆ integrin, BCMA, B7-H3, B7-H6, CAIX, CA9, FAP, FBP, fetal AchR, FRcc, GD2, GD3, Glypican-1 (GPC1), Glypican-2 (GPC2), Glypican-3 (GPC3), HLA-A1+MAGE1, HLA-A1+NY-ESO-1, IL-13Rcc2, Lewis-Y, KDR, MCSP, Mesothelin, Muc1, Muc16, NCAM, NKG2D ligands, NY-ESO-1, PRAME, PSC1, PSCA, PSMA, ROR1, ROR2, SP17, surviving, TAG72, TEMs, carcinoembryonic antigen, HMW-MAA, VEGF, CLDN18.2, neoantigen, or combinations thereof.

63. The method of claim 1, wherein the tumor antigen is derived from a cancer comprising a breast cancer, head and neck cancer, uterine cancer, brain cancer, skin cancer, renal cancer, lung cancer, colorectal cancer, prostate cancer, liver cancer, bladder cancer, kidney cancer, pancreatic cancer, thyroid cancer, esophageal cancer, eye cancer, stomach (gastric) cancer, gastrointestinal cancer, ovarian cancer, carcinoma, sarcoma, leukemia, lymphoma, myeloma, or a combination thereof.

64. The method of claim 1, wherein the IL-7 protein does not comprise a signal peptide.

65. The method of claim 1, wherein the IL-7 protein comprises: (i) amino acid residues 26-178 of SEQ ID NO: 1, (ii) amino acid residues 26-155 of SEQ ID NO: 2, (iii) amino acid residues 26-155 of SEQ ID NO: 3, (iv) amino acid residues 26-178 of SEQ ID NO: 4, (v) amino acid residues 26-177 of SEQ ID NO: 5, or (iv) amino acid residues 26-177 of SEQ ID NO: 6.


SEQUENCE LISTING (72 sequences)

SEQ ID NO: 1 - protein, 177 aa, Homo sapiens
MFHVSFRYIFGLPPLILVLLPVASSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 2 - protein, 154 aa, Rattus norvegicus
MFHVSFRYIFGIPPLILVLLPVTSSDCHIKDKDGKAFGSVLMISINQLDKMTGTDSDCPNNEPNFFKKHLCDDTKEAAFLNRAARKLRQFLKMNISEEFNDHLLRVSDGTQTLVNCTSKEEKTIKEQKKNDPCFLKRLLREIKTCWNKILKGSI

SEQ ID NO: 3 - protein, 154 aa, Mus Musculus
MFHVSFRYIFGIPPLILVLLPVTSSECHIKDKEGKAYESVLMISIDELDKMTGTDSNCPNNEPNFFRKHVCDDTKEAAFLNRAARKLKQFLKMNISEEFNVHLLTVSQGTQTLVNCTSKEEKNVKEQKKNDACFLKRLLREIKTCWNKILKGSI

SEQ ID NO: 4 - protein, 177 aa, Cercopithecus aethiops
MFHVSFRYIFGLPPLILVLLPVASSDCDIEGKDGKQYESVLMVSIDQLLDSMKEIGSNCLNNEFNFFKRHLCDDNKEGMFLFRAARKLKQFLKMNSTGDFDLHLLKVSEGTTILLNCTGKVKGRKPAALGEPQPTKSLEENKSLKEQKKLNDSCFLKRLLQKIKTCWNKILMGTKEH

SEQ ID NO: 5 - protein, 176 aa, Bos taurus
MFHVSFRYIFGIPPLILVLLPVASSDCDISGKDGGAYQNVLMVNIDDLDNMINFDSNCLNNEPNFFKKHSCDDNKEASFLNRASRKLRQFLKMNISDDFKLHLSTVSQGTLTLLNCTSKGKGRKPPSLSEAQPTKNLEENKSSKEQKKQNDLCFLKILLQKIKTCWNKILRGIKEH

SEQ ID NO: 6 - protein, 176 aa, Ovis aries
MFHVSFRYIFGIPPLILVLLPVASSDCDFSGKDGGAYQNVLMVSIDDLDNMINFDSNCLNNEPNFFKKHSCDDNKEASFLNRAARKLKQFLKMNISDDFKLHLSTVSQGTLTLLNCTSKGKGRKPPSLGEAQPTKNLEENKSLKEQRKQNDLCFLKILLQKIKTCWNKILRGITEH

SEQ ID NO: 7 - protein, 384 aa, Artificial Sequence
  amino acid sequence of human IgD constant region (Genbank accession No. P01880)
APTKAPDVFPIISGCRHPKDNSPVVLACLITGYHPTSVTVTWYMGTQSQPQRTFPEIQRRDSYYMTSSQLSTPLQQWRQGEYKCVVQHTASKSKKEIFRWPESPKAQASSVPTAQPQAEGSLAKATTAPATTRNTGRGGEEKKKEKEKEEQEERETKTPECPSHTQPLGVYLLTPAVQDLWLRDKATFTCFVVGSDLKDAHLTWEVAGKVPTGGVEEGLLERHSNGSQSQHSRLTLPRSLWNAGTSVTCTLNHPSLPPQRLMALREPAAQAPVKLSLNLLASSDPPEAASWLLCEVSGFSPPNILLMWLEDQREVNTSGFAPARPPPQPGSTTFWAWSVLRVPAPPSPQPATYTCVVSHEDSRTLLNASRSLEVSYVTDHGPMK

SEQ ID NO: 8 - protein, 327 aa, Artificial Sequence
  amino acid sequence of Partial human IgG4 constant region (Genbank accession No. AAH25985)
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 9 - protein, 245 aa, Artificial Sequence
  amino acid sequence of hyFc
RNTGRGGEEKKKEKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 245 aa, Artificial Sequence
  amino acid sequence of hyFcM1
RNTGRGGEEKKGGKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 11 - protein, 245 aa, Artificial Sequence
  amino acid sequence of hyFcM2
RNTGRGGEEKKGSKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 12 - protein, 245 aa, Artificial Sequence
  amino acid sequence of hyFcM3
RNTGRGGEEKKSGKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 13 - protein, 245 aa, Artificial Sequence
  amino acid sequence of hyFcM4
RNTGRGGEEKKSSKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 14 - protein, 243 aa, Artificial Sequence
  amino acid sequence of mouse IgG Fc variant
ASAEPRGPTIKPCPPCKCPAPNLEGGPSVFIFPPKIKDVLMISLSPIVTCVVVDVSEDDPDVQISWFVNNVEVHTAQTQTHREDYNSTLRVVSALPIQHQDWMSGKAFACAVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGKGGGNSGS

SEQ ID NO: 15 - protein, 153 aa, Artificial Sequence
  amino acid sequence of modified IL-7(M)
MDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 16 - protein, 154 aa, Artificial Sequence
  amino acid sequence of modified IL-7(MM)
MMDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 17 - protein, 155 aa, Artificial Sequence
  amino acid sequence of modified IL-7(MMM)
MMMDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 18 - protein, 155 aa, Artificial Sequence
  amino acid sequence of modified IL-7(MGM)
MGMDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 19 - protein, 155 aa, Artificial Sequence
  amino acid sequence of modified IL-7(DDD)
DDDDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 20 - protein, 156 aa, Artificial Sequence
  amino acid sequence of modified IL-7(MMMM)
MMMMDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 21 - protein, 398 aa, Artificial Sequence
  amino acid sequence of modified IL-7(M) fused hyFc
MDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHRNTGRGGEEKKKEKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 22 - protein, 399 aa, Artificial Sequence
  amino acid sequence of modified IL-7(MM) fused hyFc
MMDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHRNTGRGGEEKKKEKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 23 - protein, 400 aa, Artificial Sequence
  amino acid sequence of modified IL-7(MMM) fused hyFc
MMMDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHRNTGRGGEEKKKEKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 24 - protein, 400 aa, Artificial Sequence
  amino acid sequence of modified IL-7(MGM) fused hyFc
MGMDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHRNTGRGGEEKKKEKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 25 - protein, 401 aa, Artificial Sequence
  amino acid sequence of modified IL-7(MMMM) fused hyFc
MMMMDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHRNTGRGGEEKKKEKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 26 - protein, 397 aa, Artificial Sequence
  amino acid sequence of human IL-7 fused hyFc
DCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHRNTGRGGEEKKKEKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 27 - protein, 395 aa, Artificial Sequence
  amino acid sequence of human IL-7 fused nonlytic mouse Fc
DCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHASAEPRGPTIKPCPPCKCPAPNLEGGPSVFIFPPKIKDVLMISLSPIVTCVVVDVSEDDPDVQISWFVNNVEVHTAQTQTHREDYNSTLRVVSALPIQHQDWMSGKAFACAVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGKGGGNSGS

SEQ ID NO: 28 - DNA, 531 bp, Artificial Sequence
  nucleotide sequence of human IL-7
atgttccacgtgagcttcaggtacatcttcggcctgccacccctgatcctggtgctgctgcctgtggccagctccgactgcgacatcgagggaaaagacggcaagcagtacgaaagcgtgctgatggtgtccatcgaccagctgctggattctatgaaggagattgggagtaactgcctgaacaatgagttcaacttcttcaaacggcacatttgtgatgccaacaaggagggaatgttcctgtttcgggccgctagaaaactgaggcagttcctgaagatgaacagcaccggagactttgatctgcatctgctgaaagtgtctgagggcaccacaatcctgctgaactgcactgggcaggtgaaaggaaggaagcctgccgctctgggagaggctcagccaaccaagtcactggaggaaaacaaaagcctgaaggaacagaagaaactgaatgacctgtgctttctgaaacggctgctgcaggagatcaaaacatgttggaacaagattctgatgggcacaaaggaacac

SEQ ID NO: 29 - DNA, 534 bp, Artificial Sequence
  nucleotide sequence of modified IL-7(M)
atgttccacgtgagcttcagatacatcttcggcctgccccccctgatcctggtgctgctgcccgtggccagcagcatggactgcgacatcgagggcaaggacggcaagcagtacgagagcgtgctgatggtgagcatcgaccagctgctggacagcatgaaggagatcggcagcaactgcctgaacaacgagttcaacttcttcaagagacacatctgcgacgccaacaaggagggcatgttcctgttcagagccgccagaaagctgagacagttcctgaagatgaacagcaccggcgacttcgacctgcacctgctgaaggtgagcgagggcacaaccatcctgctgaactgcaccggccaggtgaagggcagaaagcccgccgccctgggcgaggcccagcccaccaagagcctggaggagaacaagagcctgaaggagcagaagaagctgaacgacctgtgcttcctgaagagactgctgcaggagatcaagacctgctggaacaagatcctgatgggcaccaaggagcac

SEQ ID NO: 30 - DNA, 537 bp, Artificial Sequence
  nucleotide sequence of modified IL-7(MM)
atgttccacgtgagcttcagatacatcttcggcctgccccccctgatcctggtgctgctgcccgtggccagcagcatgatggactgcgacatcgagggcaaggacggcaagcagtacgagagcgtgctgatggtgagcatcgaccagctgctggacagcatgaaggagatcggcagcaactgcctgaacaacgagttcaacttcttcaagagacacatctgcgacgccaacaaggagggcatgttcctgttcagagccgccagaaagctgagacagttcctgaagatgaacagcaccggcgacttcgacctgcacctgctgaaggtgagcgagggcacaaccatcctgctgaactgcaccggccaggtgaagggcagaaagcccgccgccctgggcgaggcccagcccaccaagagcctggaggagaacaagagcctgaaggagcagaagaagctgaacgacctgtgcttcctgaagagactgctgcaggagatcaagacctgctggaacaagatcctgatgggcaccaaggagcac

SEQ ID NO: 31 - DNA, 540 bp, Artificial Sequence
  nucleotide sequence of modified IL-7(MMM)
atgttccacgtgagcttcagatacatcttcggcctgccccccctgatcctggtgctgctgcccgtggccagcagcatgatgatggactgcgacatcgagggcaaggacggcaagcagtacgagagcgtgctgatggtgagcatcgaccagctgctggacagcatgaaggagatcggcagcaactgcctgaacaacgagttcaacttcttcaagagacacatctgcgacgccaacaaggagggcatgttcctgttcagagccgccagaaagctgagacagttcctgaagatgaacagcaccggcgacttcgacctgcacctgctgaaggtgagcgagggcacaaccatcctgctgaactgcaccggccaggtgaagggcagaaagcccgccgccctgggcgaggcccagcccaccaagagcctggaggagaacaagagcctgaaggagcagaagaagctgaacgacctgtgcttcctgaagagactgctgcaggagatcaagacctgctggaacaagatcctgatgggcaccaaggagcac

SEQ ID NO: 32 - DNA, 540 bp, Artificial Sequence
  nucleotide sequence of modified IL-7(MGM)
atgttccacgtgagcttcaggtacatcttcggcctgccacccctgatcctggtgctgctgcctgtggccagctccatggggatggactgcgacatcgagggaaaagacggcaagcagtacgaaagcgtgctgatggtgtccatcgaccagctgctggattctatgaaggagattgggagtaactgcctgaacaatgagttcaacttcttcaaacggcacatttgtgatgccaacaaggagggaatgttcctgtttcgggccgctagaaaactgaggcagttcctgaagatgaacagcaccggagactttgatctgcatctgctgaaagtgtctgagggcaccacaatcctgctgaactgcactgggcaggtgaaaggaaggaagcctgccgctctgggagaggctcagccaaccaagtcactggaggaaaacaaaagcctgaaggaacagaagaaactgaatgacctgtgctttctgaaacggctgctgcaggagatcaaaacatgttggaacaagattctgatgggcaccaaggagcac

SEQ ID NO: 33 - DNA, 540 bp, Artificial Sequence
  nucleotide sequence of modified IL-7(DDD)
atgttccacgtgagcttcagatacatcttcggcctgccccccctgatcctggtgctgctgcccgtggccagcagcgacgatgacgactgcgacatcgagggcaaggacggcaagcagtacgagagcgtgctgatggtgagcatcgaccagctgctggacagcatgaaggagatcggcagcaactgcctgaacaacgagttcaacttcttcaagagacacatctgcgacgccaacaaggagggcatgttcctgttcagagccgccagaaagctgagacagttcctgaagatgaacagcaccggcgacttcgacctgcacctgctgaaggtgagcgagggcacaaccatcctgctgaactgcaccggccaggtgaagggcagaaagcccgccgccctgggcgaggcccagcccaccaagagcctggaggagaacaagagcctgaaggagcagaagaagctgaacgacctgtgcttcctgaagagactgctgcaggagatcaagacctgctggaacaagatcctgatgggcaccaaggagcac

SEQ ID NO: 34 - DNA, 543 bp, Artificial Sequence
  nucleotide sequence of modified IL-7(MMMM)
atgttccacgtgagcttcagatacatcttcggcctgccccccctgatcctggtgctgctgcccgtggccagcagcatgatgatgatggactgcgacatcgagggcaaggacggcaagcagtacgagagcgtgctgatggtgagcatcgaccagctgctggacagcatgaaggagatcggcagcaactgcctgaacaacgagttcaacttcttcaagagacacatctgcgacgccaacaaggagggcatgttcctgttcagagccgccagaaagctgagacagttcctgaagatgaacagcaccggcgacttcgacctgcacctgctgaaggtgagcgagggcacaaccatcctgctgaactgcaccggccaggtgaagggcagaaagcccgccgccctgggcgaggcccagcccaccaagagcctggaggagaacaagagcctgaaggagcagaagaagctgaacgacctgtgcttcctgaagagactgctgcaggagatcaagacctgctggaacaagatcctgatgggcaccaaggagcac

SEQ ID NO: 35 - DNA, 1284 bp, Artificial Sequence
  nucleotide sequence of modified IL-7(M) fused hyFc
atgttccacgtgagcttcagatacatcttcggcctgccccccctgatcctggtgctgctgcccgtggccagcagcatggactgcgacatcgagggcaaggacggcaagcagtacgagagcgtgctgatggtgagcatcgaccagctgctggacagcatgaaggagatcggcagcaactgcctgaacaacgagttcaacttcttcaagagacacatctgcgacgccaacaaggagggcatgttcctgttcagagccgccagaaagctgagacagttcctgaagatgaacagcaccggcgacttcgacctgcacctgctgaaggtgagcgagggcacaaccatcctgctgaactgcaccggccaggtgaagggcagaaagcccgccgccctgggcgaggcccagcccaccaagagcctggaggagaacaagagcctgaaggagcagaagaagctgaacgacctgtgcttcctgaagagactgctgcaggagatcaagacctgctggaacaagatcctgatgggcaccaaggagcacaggaacacaggcagaggcggcgaggagaagaagaaggagaaggagaaggaggagcaggaggaaagagagaccaagacccccgagtgccccagccacacccagcccctgggcgtgttcctgttccctcccaagcccaaggacaccctgatgatcagcagaacccccgaggtgacctgcgtggtcgtggatgtgagccaggaagatcccgaagtgcagttcaactggtacgtggatggcgtggaagtgcacaacgccaagaccaagcccagagaagagcagttcaactccacctacagagtggtgagcgtgctgaccgtgctgcaccaggactggctgaacggcaaggagtacaagtgcaaggtgtccaacaaaggcctgcccagctccatcgagaagaccatcagcaaagccaaaggccagcccagagaaccccaggtgtacaccctgcctcccagccaggaagagatgaccaagaaccaggtgtccctgacctgcctggtgaaaggcttctaccccagcgacatcgccgtggagtgggaaagcaacggccagcccgagaacaattacaagacaacccctcccgtgctggatagcgatggcagcttctttctgtacagcagactgaccgtggacaagagcagatggcaggaaggcaacgtgttcagctgcagcgtgatgcacgaagccctgcacaaccactacacccagaagagcctgtccctgagcctgggcaagtgactcgagtctaga

SEQ ID NO: 36 - DNA, 1272 bp, Artificial Sequence
  nucleotide sequence of modified IL-7(MM) fused hyFc
atgttccacgtgagcttcagatacatcttcggcctgccccccctgatcctggtgctgctgcccgtggccagcagcatgatggactgcgacatcgagggcaaggacggcaagcagtacgagagcgtgctgatggtgagcatcgaccagctgctggacagcatgaaggagatcggcagcaactgcctgaacaacgagttcaacttcttcaagagacacatctgcgacgccaacaaggagggcatgttcctgttcagagccgccagaaagctgagacagttcctgaagatgaacagcaccggcgacttcgacctgcacctgctgaaggtgagcgagggcacaaccatcctgctgaactgcaccggccaggtgaagggcagaaagcccgccgccctgggcgaggcccagcccaccaagagcctggaggagaacaagagcctgaaggagcagaagaagctgaacgacctgtgcttcctgaagagactgctgcaggagatcaagacctgctggaacaagatcctgatgggcaccaaggagcacaggaacacaggcagaggcggcgaggagaagaagaaggagaaggagaaggaggagcaggaggaaagagagaccaagacccccgagtgccccagccacacccagcccctgggcgtgttcctgttccctcccaagcccaaggacaccctgatgatcagcagaacccccgaggtgacctgcgtggtcgtggatgtgagccaggaagatcccgaagtgcagttcaactggtacgtggatggcgtggaagtgcacaacgccaagaccaagcccagagaagagcagttcaactccacctacagagtggtgagcgtgctgaccgtgctgcaccaggactggctgaacggcaaggagtacaagtgcaaggtgtccaacaaaggcctgcccagctccatcgagaagaccatcagcaaagccaaaggccagcccagagaaccccaggtgtacaccctgcctcccagccaggaagagatgaccaagaaccaggtgtccctgacctgcctggtgaaaggcttctaccccagcgacatcgccgtggagtgggaaagcaacggccagcccgagaacaattacaagacaacccctcccgtgctggatagcgatggcagcttctttctgtacagcagactgaccgtggacaagagcagatggcaggaaggcaacgtgttcagctgcagcgtgatgcacgaagccctgcacaaccactacacccagaagagcctgtccctgagcctgggcaag

SEQ ID NO: 37 - DNA, 1275 bp, Artificial Sequence
  nucleotide sequence of modified IL-7(MMM) fused hyFc
atgttccacgtgagcttcagatacatcttcggcctgccccccctgatcctggtgctgctgcccgtggccagcagcatgatgatggactgcgacatcgagggcaaggacggcaagcagtacgagagcgtgctgatggtgagcatcgaccagctgctggacagcatgaaggagatcggcagcaactgcctgaacaacgagttcaacttcttcaagagacacatctgcgacgccaacaaggagggcatgttcctgttcagagccgccagaaagctgagacagttcctgaagatgaacagcaccggcgacttcgacctgcacctgctgaaggtgagcgagggcacaaccatcctgctgaactgcaccggccaggtgaagggcagaaagcccgccgccctgggcgaggcccagcccaccaagagcctggaggagaacaagagcctgaaggagcagaagaagctgaacgacctgtgcttcctgaagagactgctgcaggagatcaagacctgctggaacaagatcctgatgggcaccaaggagcacaggaacacaggcagaggcggcgaggagaagaagaaggagaaggagaaggaggagcaggaggaaagagagaccaagacccccgagtgccccagccacacccagcccctgggcgtgttcctgttccctcccaagcccaaggacaccctgatgatcagcagaacccccgaggtgacctgcgtggtcgtggatgtgagccaggaagatcccgaagtgcagttcaactggtacgtggatggcgtggaagtgcacaacgccaagaccaagcccagagaagagcagttcaactccacctacagagtggtgagcgtgctgaccgtgctgcaccaggactggctgaacggcaaggagtacaagtgcaaggtgtccaacaaaggcctgcccagctccatcgagaagaccatcagcaaagccaaaggccagcccagagaaccccaggtgtacaccctgcctcccagccaggaagagatgaccaagaaccaggtgtccctgacctgcctggtgaaaggcttctaccccagcgacatcgccgtggagtgggaaagcaacggccagcccgagaacaattacaagacaacccctcccgtgctggatagcgatggcagcttctttctgtacagcagactgaccgtggacaagagcagatggcaggaaggcaacgtgttcagctgcagcgtgatgcacgaagccctgcacaaccactacacccagaagagcctgtccctgagcctgggcaag

SEQ ID NO: 38 - DNA, 1275 bp, Artificial Sequence
  nucleotide sequence of modified IL-7(MGM) fused hyFc
atgttccacgtgagcttcaggtacatcttcggcctgccacccctgatcctggtgctgctgcctgtggccagctccatggggatggactgcgacatcgagggaaaagacggcaagcagtacgaaagcgtgctgatggtgtccatcgaccagctgctggattctatgaaggagattgggagtaactgcctgaacaatgagttcaacttcttcaaacggcacatttgtgatgccaacaaggagggaatgttcctgtttcgggccgctagaaaactgaggcagttcctgaagatgaacagcaccggagactttgatctgcatctgctgaaagtgtctgagggcaccacaatcctgctgaactgcactgggcaggtgaaaggaaggaagcctgccgctctgggagaggctcagccaaccaagtcactggaggaaaacaaaagcctgaaggaacagaagaaactgaatgacctgtgctttctgaaacggctgctgcaggagatcaaaacatgttggaacaagattctgatgggcacaaaggaacaccgcaatactgggcggggcggggaggaaaagaaaaaggagaaggaaaaggaggaacaggaggaaagagagactaagaccccagaatgtcccagccatactcagcccctgggggtgttcctgtttccccctaaacctaaggataccctgatgatcagcaggacacccgaggtgacctgcgtggtcgtggatgtgagccaggaagatcccgaagtgcagttcaactggtacgtggatggcgtggaagtgcacaacgccaagaccaagcccagagaagagcagttcaactccacctacagagtggtgagcgtgctgaccgtgctgcaccaggactggctgaacggcaaggagtacaagtgcaaggtgtccaacaaaggcctgcccagctccatcgagaagaccatcagcaaagccaaaggccagcccagagaaccccaggtgtacaccctgcctcccagccaggaagagatgaccaagaaccaggtgtccctgacctgcctggtgaaaggcttctaccccagcgacatcgccgtggagtgggaaagcaacggccagcccgagaacaattacaagacaacccctcccgtgctggatagcgatggcagcttctttctgtacagcagactgaccgtggacaagagcagatggcaggaaggcaacgtgttcagctgcagcgtgatgcacgaagccctgcacaaccactacacccagaagagcctgtccctgagcctgggcaag

SEQ ID NO: 39 - DNA, 1278 bp, Artificial Sequence
  nucleotide sequence of modified IL-7(MMMM) fused hyFc
atgttccacgtgagcttcagatacatcttcggcctgccccccctgatcctggtgctgctgcccgtggccagcagcatgatgatgatggactgcgacatcgagggcaaggacggcaagcagtacgagagcgtgctgatggtgagcatcgaccagctgctggacagcatgaaggagatcggcagcaactgcctgaacaacgagttcaacttcttcaagagacacatctgcgacgccaacaaggagggcatgttcctgttcagagccgccagaaagctgagacagttcctgaagatgaacagcaccggcgacttcgacctgcacctgctgaaggtgagcgagggcacaaccatcctgctgaactgcaccggccaggtgaagggcagaaagcccgccgccctgggcgaggcccagcccaccaagagcctggaggagaacaagagcctgaaggagcagaagaagctgaacgacctgtgcttcctgaagagactgctgcaggagatcaagacctgctggaacaagatcctgatgggcaccaaggagcacaggaacacaggcagaggcggcgaggagaagaagaaggagaaggagaaggaggagcaggaggaaagagagaccaagacccccgagtgccccagccacacccagcccctgggcgtgttcctgttccctcccaagcccaaggacaccctgatgatcagcagaacccccgaggtgacctgcgtggtcgtggatgtgagccaggaagatcccgaagtgcagttcaactggtacgtggatggcgtggaagtgcacaacgccaagaccaagcccagagaagagcagttcaactccacctacagagtggtgagcgtgctgaccgtgctgcaccaggactggctgaacggcaaggagtacaagtgcaaggtgtccaacaaaggcctgcccagctccatcgagaagaccatcagcaaagccaaaggccagcccagagaaccccaggtgtacaccctgcctcccagccaggaagagatgaccaagaaccaggtgtccctgacctgcctggtgaaaggcttctaccccagcgacatcgccgtggagtgggaaagcaacggccagcccgagaacaattacaagacaacccctcccgtgctggatagcgatggcagcttctttctgtacagcagactgaccgtggacaagagcagatggcaggaaggcaacgtgttcagctgcagcgtgatgcacgaagccctgcacaaccactacacccagaagagcctgtccctgagcctgggcaag

SEQ ID NO: 40 - protein, 4 aa, Artificial Sequence
  synthetic oligopeptide fragment
MMMM

SEQ ID NO: 41 - protein, 4 aa, Artificial Sequence
  synthetic oligopeptide fragment
MGGM

SEQ ID NO: 42 - protein, 4 aa, Artificial Sequence
  synthetic oligopeptide fragment
MMGG

SEQ ID NO: 43 - protein, 4 aa, Artificial Sequence
  synthetic oligopeptide fragment
GGMM

SEQ ID NO: 44 - protein, 4 aa, Artificial Sequence
  synthetic oligopeptide fragment
MGMG

SEQ ID NO: 45 - protein, 4 aa, Artificial Sequence
  synthetic oligopeptide fragment
GMMG

SEQ ID NO: 46 - protein, 4 aa, Artificial Sequence
  synthetic oligopeptide fragment
GGGM

SEQ ID NO: 47 - protein, 4 aa, Artificial Sequence
  synthetic oligopeptide fragment
MGGG

SEQ ID NO: 48 - protein, 4 aa, Artificial Sequence
  synthetic oligopeptide fragment
GMGG

SEQ ID NO: 49 - protein, 4 aa, Artificial Sequence
  synthetic oligopeptide fragment
GGMG

SEQ ID NO: 50 - protein, 5 aa, Artificial Sequence
  synthetic oligopeptide fragment
GGMMM

SEQ ID NO: 51 - protein, 5 aa, Artificial Sequence
  synthetic oligopeptide fragment
GGGMM

SEQ ID NO: 52 - protein, 5 aa, Artificial Sequence
  synthetic oligopeptide fragment
GGGGM

SEQ ID NO: 53 - protein, 5 aa, Artificial Sequence
  synthetic oligopeptide fragment
MGMMM

SEQ ID NO: 54 - protein, 5 aa, Artificial Sequence
  synthetic oligopeptide fragment
MGGMM

SEQ ID NO: 55 - protein, 5 aa, Artificial Sequence
  synthetic oligopeptide fragment
MGGGM

SEQ ID NO: 56 - protein, 5 aa, Artificial Sequence
  synthetic oligopeptide fragment
MMGMM

SEQ ID NO: 57 - protein, 5 aa, Artificial Sequence
  synthetic oligopeptide fragment
MMGGM

SEQ ID NO: 58 - protein, 5 aa, Artificial Sequence
  synthetic oligopeptide fragment
MMGGG

SEQ ID NO: 59 - protein, 5 aa, Artificial Sequence
  synthetic oligopeptide fragment
MMMGM

SEQ ID NO: 60 - protein, 5 aa, Artificial Sequence
  synthetic oligopeptide fragment
MGMGM

SEQ ID NO: 61 - protein, 5 aa, Artificial Sequence
  synthetic oligopeptide fragment
GMGMG

SEQ ID NO: 62 - protein, 5 aa, Artificial Sequence
  synthetic oligopeptide fragment
GMMMG

SEQ ID NO: 63 - protein, 5 aa, Artificial Sequence
  synthetic oligopeptide fragment
GGMGM

SEQ ID NO: 64 - protein, 5 aa, Artificial Sequence
  synthetic oligopeptide fragment
GGMMG

SEQ ID NO: 65 - protein, 5 aa, Artificial Sequence
  synthetic oligopeptide fragment
GMMGM

SEQ ID NO: 66 - protein, 5 aa, Artificial Sequence
  synthetic oligopeptide fragment
MGMMG

SEQ ID NO: 67 - protein, 5 aa, Artificial Sequence
  synthetic oligopeptide fragment
GMGGM

SEQ ID NO: 68 - protein, 5 aa, Artificial Sequence
  synthetic oligopeptide fragment
MMGMG

SEQ ID NO: 69 - protein, 5 aa, Artificial Sequence
  synthetic oligopeptide fragment
GMMGG

SEQ ID NO: 70 - protein, 5 aa, Artificial Sequence
  synthetic oligopeptide fragment
GMGGG

SEQ ID NO: 71 - protein, 5 aa, Artificial Sequence
  synthetic oligopeptide fragment
GGMGG

SEQ ID NO: 72 - protein, 5 aa, Artificial Sequence
  synthetic oligopeptide fragment
GGGGG